(19) United States
(12) Patent Application Publication
(10) Pub. No.: US 2018/0263925 A1
(43) Pub. Date: Sep. 20, 2018

(54) METHODS FOR THE TREATMENT OF ALOPECIA AREATA UTILIZING GENE MODULATION APPROACHES

(71) Applicant: RXi Pharmaceuticals Corporation, Marlborough, MA (US)

(72) Inventors: Gerard Cauwenbergh, Plainsboro, NJ (US); Keith Johnson, Durham, NC (US)

(73) Assignee: RXi Pharmaceuticals Corporation, Marlborough, MA (US)

(21) Appl. No.: 15/532,804

(22) PCT Filed: Dec. 3, 2015
(86) PCT No.: PCT/US15/63805

§ 371 Date: Jun. 2, 2017

Related U.S. Application Data
(60) Provisional application No. 62/095,309, filed on Dec. 22, 2014.
(60) Provisional application No. 62/087,138, filed on Dec. 3, 2014.

(51) Int. Cl. A61K 31/122; C12N 15/113; A61K 9/00; A61K 47/54; A61K 31/713; A61P 17/14

(52) U.S. Cl. CPC A61K 31/122; C12N 15/1136; C12N 15/1138; A61K 9/0014; A61K 47/554; A61K 47/54; C12N 2310/351; A61K 31/713; A61P 17/14; C12N 2310/14; C12N 2310/315; C12N 2310/321; C12N 2310/32; A61K 47/545

(57) ABSTRACT

The present invention relates to methods and compositions for the treatment of alopecia areata. In some aspects, the present invention relates to haptens for use in treating alopecia areata. In other aspects, the present invention relates to RNAi constructs with improved tissue and cellular uptake characteristics and methods of use of these compounds the treatment of alopecia areata. In other aspects, the present invention relates to compositions comprising haptens formulated as gels or ointments.

### RELATED APPLICATIONS

This application claims the benefit under 35 U.S.C. § 119(e) of U.S. Provisional Application Ser. No. 62/087,138, entitled “METHODS FOR THE TREATMENT OF ALOPECIA AREATA UTILIZING GENE MODULATION APPROACHES,” filed on Dec. 3, 2014, and U.S. Provisional Application Ser. No. 62/095,309, entitled “METHODS FOR THE TREATMENT OF ALOPECIA AREATA UTILIZING GENE MODULATION APPROACHES,” filed on Dec. 22, 2014, the entire disclosures of each of which are herein incorporated by reference in their entireties.

### FIELD OF THE INVENTION

The invention pertains to the use of two therapeutic approaches to treat alopecia areata. The first is a non-targeted approach to reduce the expression of multiple genes of interest utilizing a small molecule hapten. Alternatively, a targeted approach to specifically silence up-regulated genes of interest utilizing nucleic acid molecules with improved in vivo delivery properties may be utilized. These therapeutic approaches may be used in combination with each other or separately.

### BACKGROUND

Alopecia areata (AA) is an autoimmune disease that involves the partial loss of hair on the scalp, full loss on the scalp (totalis), or full loss of hair on the body (universalis). Although the precise pathology of the disease is unknown, genetic, immunologic and environmental factors, such as viral infections, have been demonstrated to play a role in the development of AA. The growth cycle of a hair follicle occurs in three stages: anagen phase (active growth stage), catagen phase (short transition phase at the end of the anagen phase, signaling the end of the active growth phase) and telogen phase (resting phase). The hair follicle contains its own immunosuppressive microenvironment during the anagen phase which results in reduced immune stimulation due to reduced levels of major histocompatibility complex (MHC) class I molecules, termed the “hair follicle immune privilege”. In AA, the hair follicle immune privilege is impaired, leading to an autoimmune response against hair follicle autoantigens, resulting in the loss of hair.

Complementary oligonucleotide sequences are promising therapeutic agents and useful research tools in elucidating gene functions. However, prior art oligonucleotide molecules suffer from several problems that may impede their clinical development, and frequently make it difficult to achieve intended efficient inhibition of gene expression (including protein synthesis) using such compositions in vivo.

A major problem has been the delivery of these compounds to cells and tissues. Conventional double-stranded RNAi compounds, 19-29 bases long, form a highly negatively-charged rigid helix of approximately 1.5 by 10-15 nm in size. This rod type molecule cannot get through the cell-membrane and as a result has very limited efficacy both in vitro and in vivo. As a result, all conventional RNAi compounds require some kind of delivery vehicle to promote their tissue distribution and cellular uptake. This is considered to be a major limitation of the RNAi technology.

There have been previous attempts to apply chemical modifications to oligonucleotides to improve their cellular uptake properties. One such modification was the attachment of a cholesterol molecule to the oligonucleotide. A first report on this approach was by Letsinger et al., in 1989. Subsequently, ISIS Pharmaceuticals, Inc. (Carlsbad, Calif.) reported on more advanced techniques in attaching the cholesterol molecule to the oligonucleotide (Manoharan, 1992).

With the discovery of siRNAs in the late nineties, similar types of modifications were attempted on these molecules to enhance their delivery profiles. Cholesterol molecules conjugated to slightly modified (Soutschek, 2004) and heavily modified (Wolfrum, 2007) siRNAs appeared in the literature. Yamada et al., 2008 also reported on the use of advanced linker chemistries which further improved cholesterol mediated uptake of siRNAs. In spite of all this effort, the uptake of these types of compounds impaired to be inhibited in the presence of biological fluids resulting in highly limited efficacy in gene silencing in vivo, limiting the applicability of these compounds in a clinical setting.

### SUMMARY

In some aspects, the disclosure relates to a method for treating alopecia areata comprising administering to a subject in need thereof a therapeutically effective amount of a hapten that reduces the expression of a gene encoding and/or a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

In some embodiments, the hapten is DPCP, imiquimod, ingenol mebutate, or SADBE. In some embodiments, the hapten is DPCP. In some embodiments, a therapeutically effective amount of DPCP is used to reduce levels of Tbx21 for treating alopecia areata.

In some embodiments, the hapten is formulated in a composition comprising a gel formulation.

In some embodiments, a low sensitizing dose of the composition is administered to a first site on the skin of the subject, followed by a subsequent administration of a challenge dose of the composition to a second site on the skin of the subject, wherein the composition comprises DPCP.

In some embodiments, the low sensitizing dose is about 0.1 to about 1% DPCP, and wherein the challenge dose is 0.0000001% to about 0.4% DPCP. In some embodiments, the sensitizing dose is 0.4% DPCP.

In some embodiments, the challenge dose is administered to the skin daily. In other embodiments, the challenge dose is administered to the skin every other day. In another embodiment, the challenge dose is administered to the skin twice a week. In some embodiments, the challenge dose is administered to the skin weekly. In other embodiments, the challenge dose is administered to the skin every two weeks. In another embodiment, the challenge dose is administered to the skin every three weeks. In some embodiments, the challenge dose is administered to the skin in any combination of daily, twice a week, weekly, every other week, every three weeks and/or monthly.

In some embodiments, the composition comprises DPCP.

In some embodiments, the composition comprises a) a first co-solvent comprising a non-ionic surfactant; b) a second co-solvent comprising an alcoholic ester; and, c) a gelling agent.

In some embodiments, the first co-solvent is selected from the group consisting of polyoxyethylene (20) monoleate, polyoxyethylene (20) sorbitan monooleate, polysorbate 80, palmitate and stearate, wherein the second co-solvent is selected from the group consisting of isopropyl myristate and isopropyl palmitate, and wherein the gelling agent is selected from the group consisting of polyoxyl 40 stearate and hydroxypropyl cellulose.

In some aspects, the disclosure relates to a method for treating alopecia areata comprising administering to a subject in need thereof a therapeutically effective amount of at least one nucleic acid molecule that is directed against a gene encoding a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

In some embodiments, the nucleic acid molecule is a chemically modified oligonucleotide. In some embodiments, the nucleic acid molecule is a double stranded nucleic acid molecule. In some embodiments, the nucleic acid molecule is an isolated double stranded nucleic acid molecule that includes a double stranded region and a single stranded region, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the isolated double stranded nucleic acid molecule are modified.

In some embodiments, the isolated double stranded nucleic acid molecule further comprises a hydrophobic conjugate that is attached to the isolated double stranded nucleic acid molecule.

In some embodiments, the nucleic acid molecule is directed against a gene encoding Tbx21. In some embodiments, the nucleic acid molecule is directed against a gene encoding CTGF. In some embodiments, the nucleic acid molecule silences gene expression through an RNAi mechanism of action. In another embodiment, the nucleic acid molecule is in a composition formulated for topical delivery. In some embodiments, the nucleic acid molecule is in a composition formulated for delivery to the skin. In some embodiments, the nucleic acid molecule is in a composition formulated for intradermal injection.

In some embodiments, the nucleic acid molecule is in a composition formulated for extended release of the molecule following intradermal injection.

In some embodiments, two or more nucleic acid molecules directed against genes encoding different proteins are administered to the subject. In some embodiments, wherein two or more nucleic acid molecules directed against genes encoding the same protein are administered to the subject. In some embodiments, the nucleic acid molecule is composed of nucleotides and at least 30% of the nucleotides are chemically modified. In some embodiments, wherein the nucleic acid molecule contains at least one modified backbone linkage.

In some embodiments, wherein the nucleic acid molecule contains at least one phosphorothioate linkage. In some embodiments, the nucleic acid molecule is composed of nucleotides and at least one of the nucleotides contains a 2′ chemical modification selected from the group consisting of 2′OMe and 2′Fluoro. In some embodiments, the nucleic acid molecule is administered once. In some embodiments, the nucleic acid molecule is administered more than once.

In some embodiments, the nucleic acid molecule comprises at least 12 contiguous nucleotides of a sequence as set forth in SEQ ID NO.: 17. In some embodiments, the nucleic acid molecule is directed against at least 12 contiguous nucleotides of a sequence as set forth in SEQ ID NO.: 24.

In some aspects, the disclosure relates to a method for treating alopecia areata comprising administering to a subject in need thereof a therapeutically effective amount of a hapten that reduces the expression of a gene encoding and/or a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF and a therapeutically effective amount of at least one nucleic acid molecule that is directed against a gene encoding a molecule selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

In some embodiments, the hapten is DPCP, imiquimod, ingenol mebutate, or SADBE.

In some embodiments, the hapten and the nucleic acid are administered separately. In some embodiments, the hapten and the nucleic acid are administered at the same time. In some embodiments, the hapten and the nucleic acid are administered in the same formulation. In some embodiments, the administration of the hapten and the nucleic acid is temporally separate.

In some embodiments, the hapten is formulated in a composition comprising an ointment formulation.

In some embodiments, a low sensitizing dose of the composition is administered to a first site on the skin of the subject, followed by a subsequent administration of a challenge dose of the composition to a second site on the skin of the subject, wherein the composition comprises DPCP.

In some embodiments, the low sensitizing dose is about 0.1 to about 1% DPCP, and wherein the challenge dose is 0.0000001% to about 0.4% DPCP. In some embodiments, the sensitizing dose is 0.4% DPCP.

In some embodiments, the challenge dose is administered to the skin daily. In some embodiments, the challenge dose is administered to the skin every other day. In another embodiment, the challenge dose is administered to the skin twice a week. In some embodiments, the challenge dose is administered to the skin weekly. In another embodiment, the challenge dose is administered to the skin every two weeks. In another embodiment, the challenge dose is administered to the skin every three weeks. In some embodiments, said challenge dose is administered to the skin in any combination of daily, twice a week, weekly, every other week, every three weeks and/or monthly.

In some embodiments, the composition comprises DPCP.

In some embodiments, the composition comprises a) a first co-solvent comprising a non-ionic surfactant; b) a second co-solvent comprising an alcoholic ester; and, c) a thickening agent.

In some embodiments, the first co-solvent is selected from the group consisting of polyoxyethylene (20) monoleate, polyoxyethylene (20) sorbitan monooleate, polysorbate 80, palmitate and stearate, wherein the second co-solvent is selected from the group consisting of isopropyl myristate and isopropyl palmitate, and wherein the thickening agent is selected from the group consisting of white wax, cetyl ester wax and glyceryl monosterate.

In some aspects, the disclosure relates to a composition comprising a hapten gel formulation, wherein the composition comprises a) a first co-solvent comprising a non-ionic surfactant, b) a second co-solvent comprising an alcoholic ester, and c) a gelling agent.

In some embodiments, said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

In some embodiments, the composition comprises 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol.

In some embodiments, the hapten is DPCP, imiquimod, ingenol mebutate or SADBE. In some embodiments, the hapten is DPCP.

In some aspects, the disclosure relates to a composition comprising a hapten ointment formulation, wherein the composition comprises a) a first co-solvent comprising a non-ionic surfactant, b) a second co-solvent comprising an alcoholic ester, and c) a thickening agent.

In some embodiments, said first co-solvent is selected from the group consisting of polyoxyethylene (20) monoleate, polyoxyethylene (20) sorbitan monooleate, polysorbate 80, palmitate and stearate, wherein said second co-solvent is selected from the group consisting of isopropyl myristate and isopropyl palmitate, and wherein said thickening agent is selected from the group consisting of white wax, cetyl ester wax and glyceryl monosterate.

In some aspects, the disclosure relates to a composition comprising a hapten ointment formulation, wherein the composition comprises 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

In some embodiments, the hapten is DPCP, imiquimod, ingenol mebutate or SADBE. In some embodiments, the hapten is DPCP

In some embodiments, the dose of DPCP is 0.0000001% to about 1%.

In some aspects, the disclosure relates to a method for treating alopecia areata comprising administering to a subject in need thereof a therapeutically effective amount of a composition comprising a hapten gel formulation, wherein the composition comprises a) a first co-solvent comprising a non-ionic surfactant, b) a second co-solvent comprising an alcoholic ester, and c) a gelling agent.

In some embodiments, said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

In some embodiments, the gel composition is comprised of 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol. In some embodiments, the hapten is DPCP, imiquimod, ingenol mebutate or SADBE. In some embodiments, the hapten is DPCP.

In some aspects, the disclosure relates to a method for treating alopecia areata comprising administering to a subject in need thereof a therapeutically effective amount of a composition comprising a hapten ointment formulation, wherein the composition comprises a) a first co-solvent comprising a non-ionic surfactant, b) a second co-solvent comprising an alcoholic ester, and c) a thickening agent.

In some embodiments, the first co-solvent is selected from the group comprising polyoxyethylene (20) monoleate, polyoxyethylene (20) sorbitan monooleate, polysorbate 80, palmitate and stearate, and wherein said second co-solvent is selected from the group comprising of isopropyl myristate and isopropyl palmitate, and wherein said thickening agent is selected from the group comprising of white wax, cetyl ester wax and glyceryl monosterate.

In some aspects, the disclosure relates to a method for treating alopecia areata comprising administering to a subject in need thereof a therapeutically effective amount of a composition comprising a hapten ointment formulation, wherein the ointment is comprised of 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

In some embodiments, the hapten is DPCP, imiquimod, ingenol mebutate or SADBE. In some embodiments, the hapten is DPCP.

In some embodiments, the disclosure relates to a method for treating alopecia areata comprising administering to a subject in need thereof a therapeutically effective amount of a gel or ointment composition described herein, wherein the hapten is DPCP and wherein the dose of DPCP is about 0.0000001% to about 1%.

Aspects of the invention relate to methods comprising administering any of the compositions described herein to a subject in need thereof.

Multiple synergies can exist between the nucleic acids described herein and the haptens described herein. The mechanism of action of the haptens is linked to the hapten's ability to alter the expression of multiple genes and miRNAs involved in the immune response. These gene targets may be modulated by an RNAi approach, utilizing the nucleic acids (i.e. sd-rxRNAs), to further enhance the haptens efficacy and response rates.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE FIGURES

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic graph showing the stability of DPCP in various solvents as determined by reverse phase HPLC.

FIG. 2 is a schematic graph showing a DPCP assay after 12 days at 50° C. The stability of DPCP in solvents was determined using reverse phase HPLC on a C18 column.

### DETAILED DESCRIPTION

### Haptens

As used herein, the term “hapten” refers to a molecule that can bind to a protein, such as an endogenous protein, to create a complete antigen that evokes contact hypersensitivity (CHS). Non-limiting examples of haptens include Dinitrochlorobenzene (DNCB), Squaric Acid Dibutylester (SADBE), Diphenylcyclopropenone (DPCP), Imiquimod and Ingenol mebutate. CHS clinically manifests as allergic contact dermatitis (ACD). Without wishing to be bound by any theory, this may be achieved via the mechanism of delayed-type (Type IV) hypersensitivity (DTH). In DTH, the antigen that enters the skin (or the hapten-peptide complex formed after hapten entry) is captured by epidermal Langerhans cells or dermal dendritic cells.

This interaction begins a process of tethering, rolling, firm adhesion and diapedesis that culminates in extravasation of the T-cell; this cell is then guided to the antigen by chemokines produced by local skin cells, and in particular CTACK/CCL27, a skin-limited chemokine ligand to chemokine receptor CCR10 produced by basal keratinocytes and upregulated with cutaneous inflammation (see Levis et al., Topical immunotherapy of basal cell carcinomas with dinitrochlorobenzene; Cancer Res. 1973; 33:3036-42, herein incorporated by reference in its entirety). Initial exposure to the hapten produces an induction phase, which is generally subclinical; further contact (even with far lower doses) after up to ten days of effector T-cell expansion produces an elicitation phase, characterized by overt dermal inflammation (see Levis et al. Lymphokine production in cell mediated allergic dermatitis, Lancet 2: 389-390, 1973, herein incorporated by reference in its entirety).

Diphencyprone or Diphenylcyclopropenone (DPCP)

DPCP is a potent contact sensitizer that has distinct advantages for therapeutic use. The standard dose administered to a subject, 2.0% DPCP, is an overdose which causes the subject to become overly hypersensitized to the hapten during challenge. As a result of the sensitizing overdose, in earlier embodiments, the challenge doses had to be very low, 0.002% DPCP, due to hypersensitization. Recently, Levis et al. (US Patent Publication No. US 2011/0268761 A1, herein incorporated by reference in its entirety) demonstrated that a low sensitizing dose of about 0.4% DPCP gel compared to the standard sensitizing dose of 2.0% DPCP used in the art prevents the subject from becoming overly hypersensitive to the challenge dose. Lowering the sensitization dose allows for significantly higher challenge doses since the 0.4% sensitization dose does not overly hypersensitize the subject to the challenge dose. Also, a 0.4% sensitization dose allows for more frequent repeated application of the challenge dose (0.04%) which significantly enhances the immune response to DPCP. Avoidance of hyper-sensitization in patients to the challenge doses results in an improved safety and tolerability profile and a more robust therapeutic effect.

Treatment of alopecia areata patients with DPCP (using a 2% sensitizing dose followed by 0.001% challenge doses) and SADBE has been reported (Alopecia Areata: Treatment of Today and Tomorrow. Freyschmidt-Paul et al, J Invest Dermatol Vol 8 No 1 Jun. 2003, herein incorporated by reference in its entirety). Following treatment with DPCP, initial hair regrowth was visible after 8-12 weeks with response rates of 29-78% in over 25 different studies for treating AA with haptens.

Imiquimod

Imiquimod is a small molecule immune response modifier that works through toll-like receptor 7 (TLR-7) to activate Langerhans cells, natural killer cells, macrophages and B-lymphocytes.

Ingenol Mebutate

Ingenol mebutate is a naturally isolated small molecule from the plant Euphorbia peplus used in the treatment of actinic keratoses.

Dinitrochlorobenzene (DNCB)

DNCB is a potent contact sensitizer that has been shown to stimulate the release of CD4+ helper T-cells and induce TH-1 type immunity by releasing cytokines, including Interleukin-2.

Squaric Acid Dibutylester (SADBE)

SADBE is a contact sensitizer that augments, stimulates, activates, potentiates, or modulates the immune response at either the cellular or humoral level. Its mode of action is either non-specific, resulting in increased immune responsiveness to a wide variety of antigens, or antigen-specific, i.e., affecting a restricted type of immune response to a narrow group of antigens. The therapeutic efficacy is related to its antigen-specific immunoadjuvanticity.

As used herein, the term “therapeutically effective amount” refers to an amount that provides a therapeutic or prophylactic benefit.

### Compositions of Haptens

The disclosure provides compositions of haptens that are useful in the treatment of alopecia areata. Thus, in one aspect, the present disclosure provides compositions comprising a hapten. In some embodiments, the hapten elicits a T-cell response. In some embodiments, the hapten is selected from diphenylcyclopropenone (DPCP), imiquimod, ingenol mebutate, and Squaric Acid Dibutylester (SADBE). In certain particular embodiments, the hapten is DPCP.

In some embodiments, the hapten is formulated in a composition comprising a gel formulation. In some embodiments, the composition comprises (a) a non-ionic surfactant, (b) an alcoholic ester, and (c) a gelling agent. In some embodiments, the non-ionic surfactant is selected from polyoxyethylene (20) monoleate, polyoxyethylene (20) sorbitan monooleate, polysorbate 80, palmitate, and stearate. In certain particular embodiments, the non-ionic surfactant is polysorbate 80. In some embodiments, the alcoholic ester is selected from isopropyl myristate and isopropyl palmitate. In certain particular embodiments, the alcoholic ester is isopropyl myristate. In some embodiments, the gelling agent is polyoxyl 40 stearate. Thus, in some embodiments, the composition comprises a hapten, a non-ionic surfactant selected from polyoxyethylene (20) monoleate, polyoxyethylene (20) sorbitan monooleate, polysorbate 80, palmitate and stearate; an alcoholic ester selected from isopropyl myristate and isopropyl palmitate; and a gelling agent that is polyoxyl 40 stearate. In certain particular embodiments, the composition comprises a hapten, polysorbate 80, isopropyl myristate, and polyoxyl 40 stearate. In one particular embodiment, the composition is a formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some embodiments, a hapten, such as DPCP, is formulated in a composition comprising a gel formulation comprising a) a first co-solvent comprising a non-ionic surfactant, b) a second co-solvent comprising an alcoholic ester, and c) a gelling agent. The first co-solvent is selected from the group consisting of polyoxyethylene (20) monoleate, polyoxyethylene (20) sorbitan monooleate, palmitate and stearate, wherein the second co-solvent is selected from the group consisting of isopropyl myristate and isopropyl palmitate, and wherein said gelling agent is polyoxyl 40 stearate.

Alternatively, the gel can be comprised of a) a first co-solvent comprising a non-ionic surfactant, b) a second co-solvent comprising an alcoholic ester, c) an alcohol and d) a thickening agent. The first co-solvent can be selected from the group consisting of polyoxyethylene (20) monoleate, polyoxyethylene (20) sorbitan monooleate, polysorbate 80 (PS80), palmitate and stearate, wherein the second co-solvent can be selected from the group consisting of isopropyl myristate and isopropyl palmitate, wherein the alcohol can be selected from the group consisting of ethanol or isopropanol and wherein the gelling agent is hydroxypropyl cellulose (Klucel™).

In other embodiments, the hapten, such as DPCP, is formulated in a composition comprising an ointment formulation. The ointment can comprise a) a first co-solvent comprising a non-ionic surfactant, b) a second co-solvent comprising an alcoholic ester, and c) a thickening agent. The first co-solvent can be selected from the group consisting of polyoxyethylene (20) monoleate, polyoxyethylene (20) sorbitan monooleate, palmitate and stearate, wherein the second co-solvent can be selected from the group consisting of isopropyl myristate and isopropyl palmitate, and wherein the thickening agent can be selected from the group consisting of and/or any combination of white wax, cetyl ester wax and/or glyceryl monostearate.

In other embodiments, the hapten, such as DPCP, is formulated as a cream, lotion, foam, patch or paste.

The compositions can contain one or more haptens at any therapeutically effective amount. In some embodiments, the composition may comprise a sensitizing dose of hapten. In certain of the embodiments described herein, the composition comprises from about 0.1% to about 1% hapten. In some embodiments, the composition comprises at least 0.1%, at least 0.2%, at least 0.3%, at least 0.4%, at least 0.5%, at least 0.6%, at least 0.7%, at least 0.8%, at least 0.9% or at least 1% hapten. In certain particular embodiments, the composition comprises 0.4% hapten. In other embodiments, the composition may comprise a challenge dose. In certain of the embodiments described herein, the composition comprises from about 0.0000001% to about 0.4% hapten. In some embodiments the hapten is selected from diphenylcyclopropenone (DPCP), imiquimod, ingenol mebutate, and Squaric Acid Dibutylester (SADBE). In certain particular embodiments, the hapten is DPCP.

In some embodiments, the gel formulation containing a hapten can comprise one or more of the following excipients: BHT, Klucel MF Pharm, isopropyl alcohol, propylene glycol, polysorbate 80, and/or isopropyl myristate. In some embodiments, the percentages w/w of these excipients correspond to approximately 0.1%, 2%, 57.9%, 10%, 15%, and 15%, respectively. In some embodiments, the excipients are reduced slightly in formulations containing DPCP.

In some embodiments, the ointment formulation containing a hapten can comprise one or more of the following excipients: BHT, methylparaben, propylparaben, cetyl esters wax, white wax, polysorbate 80, and isopropyl myristate. In some embodiments, the percentages w/w of these excipients corresponds to approximately 0.1%, 0.1%, 0.05%, 10%, 10%, 39.875%, and 39.875%, respectively. In some embodiments, the excipients are reduced slightly in formulations containing DPCP.

In some embodiments, the ointment formulation containing a hapten can comprise one or more of the following excipients: BHT, methylparaben, propylparaben, glyceryl monostearate, EP, cetyl esters wax, white wax, polysorbate 80, and isopropyl myristate. In some embodiments, the percentages w/w of these excipients correspond to 0.1%, 0.1%, 0.05%, 5%, 7.5%, 7.5%, 39.875%, and 39.875%, respectively. In some embodiments, the excipients are reduced slightly in formulations containing DPCP.

### Nucleic Acid Molecules

As used herein, “nucleic acid molecule” includes but is not limited to: sd-rxRNA, rxRNAori, oligonucleotides, ASO, siRNA, shRNA, miRNA, ncRNA, cp-lasiRNA, aiRNA, RXI-109, single-stranded nucleic acid molecules, double-stranded nucleic acid molecules, RNA and DNA. In some embodiments, the nucleic acid molecule is a chemically modified nucleic acid molecule, such as a chemically modified oligonucleotide.

sd-rxRNA Molecules

Aspects of the invention relate to sd-rxRNA molecules. As used herein, an “sd-rxRNA” or an “sd-rxRNA molecule” refers to a self-delivering RNA molecule such as those described in, and incorporated by reference from, U.S. Pat. No. 8,796,443, granted on Aug. 5, 2014, entitled “REDUCED SIZE SELF-DELIVERING RNAI COMPOUNDS” and PCT Publication No. WO2010/033247 (Application No. PCT/US2009/005247), filed on Sep. 22, 2009, and entitled “REDUCED SIZE SELF-DELIVERING RNAI COMPOUNDS.” Briefly, an sd-rxRNA, (also referred to as an sd-rxRNA^{nano}) is an isolated asymmetric double stranded nucleic acid molecule comprising a guide strand, with a minimal length of 16 nucleotides, and a passenger strand of 8-18 nucleotides in length, wherein the double stranded nucleic acid molecule has a double stranded region and a single stranded region, the single stranded region having 4-12 nucleotides in length and having at least three nucleotide backbone modifications. In preferred embodiments, the double stranded nucleic acid molecule has one end that is blunt or includes a one or two nucleotide overhang. sd-rxRNA molecules can be optimized through chemical modification, and in some instances through attachment of hydrophobic conjugates.

In some embodiments, an sd-rxRNA comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified.

The polynucleotides of the invention are referred to herein as isolated double stranded or duplex nucleic acids, oligonucleotides or polynucleotides, nano molecules, nano RNA, sd-rxRNA^{nano}, sd-rxRNA or RNA molecules of the invention.

sd-rxRNAs are much more effectively taken up by cells compared to conventional siRNAs. These molecules are highly efficient in silencing of target gene expression and offer significant advantages over previously described RNAi molecules including high activity in the presence of serum, efficient self delivery, compatibility with a wide variety of linkers, and reduced presence or complete absence of chemical modifications that are associated with toxicity.

In contrast to single-stranded polynucleotides, duplex polynucleotides have traditionally been difficult to deliver to a cell as they have rigid structures and a large number of negative charges which makes membrane transfer difficult. sd-rxRNAs however, although partially double-stranded, are recognized in vivo as single-stranded and, as such, are capable of efficiently being delivered across cell membranes. As a result the polynucleotides of the invention are capable in many instances of self delivery. Thus, the polynucleotides of the invention may be formulated in a manner similar to conventional RNAi agents or they may be delivered to the cell or subject alone (or with non-delivery type carriers) and allowed to self deliver. In one embodiment of the present invention, self delivering asymmetric double-stranded RNA molecules are provided in which one portion of the molecule resembles a conventional RNA duplex and a second portion of the molecule is single stranded.

The oligonucleotides of the invention in some aspects have a combination of asymmetric structures including a double stranded region and a single stranded region of 5 nucleotides or longer, specific chemical modification patterns and are conjugated to lipophilic or hydrophobic molecules. This class of RNAi like compounds have superior efficacy in vitro and in vivo. It is believed that the reduction in the size of the rigid duplex region in combination with phosphorothioate modifications applied to a single stranded region contribute to the observed superior efficacy.

Methods of effectively administering sd-rxRNA to the skin and silencing gene expression have been demonstrated in U.S. Pat. No. 8,664,189, granted on Mar. 4, 2014 and entitled “RNA INTERFERENCE IN SKIN INDICATIONS,” US Patent Publication No. US2014/0113950, filed on Apr. 4, 2013 and entitled “RNA INTERFERENCE IN DERMAL AND FIBROTIC INDICATIONS,” PCT Publication No. WO 2010/033246, filed on Sep. 22, 2009 and entitled “RNA INTERFERENCE IN SKIN INDICATIONS” and PCT Publication No. WO2011/119887, filed on Mar. 24, 2011 and entitled “RNA INTERFERENCE IN DERMAL AND FIBROTIC INDICATIONS.” Each of the above-referenced patents and publications are incorporated by reference herein in their entireties.

For example, FIG. 42 in US Patent Publication No. US2014/0113950 demonstrates CTGF silencing following intradermal injection of RXI-109 in vivo (Rat skin) after two intradermal injections of RXI-109 (CTGF-targeting sd-rxRNA). Data presented are from a study using an excisional wound model in rat dermis. Following two intradermal injections of RXI-109, silencing of CTGF vs. non-targeting control was sustained for at least five days. The reduction of CTGF mRNA was dose dependent: 51 and 67% for 300 and 600 μg, respectively, compared to the dose matched non-targeting control. Methods: RXI-109 or non-targeting control (NTC) was administered by intradermal injection (300 or 600 ug per 200 uL injection) to each of four sites on the dorsum of rats on Days 1 and 3. A 4 mm excisional wound was made at each injection site ˜30 min after the second dose (Day 3). Terminal biopsy samples encompassing the wound site and surrounding tissue were harvested on Day 8. RNA was isolated and subjected to gene expression analysis by qPCR. Data are normalized to the level of the TATA box binding protein (TBP) housekeeping gene and graphed relative to the PBS vehicle control set at 1.0. Error bars represent standard deviation between the individual biopsy samples. P values for RXI-109-treated groups vs dose-mathced non-targeting control groups were ** p<0.001 for 600 μg, * p<0.01 for 300 μg.

It should be appreciated that the sd-rxRNA molecules disclosed herein can be administered to the skin in the same manner as the sd-rxRNA molecules disclosed in US Patent Publication No. US2014/0113950, incorporated by reference in its entirety.

In a preferred embodiment the RNAi compounds of the invention comprise an asymmetric compound comprising a duplex region (required for efficient RISC entry of 8-15 bases long) and single stranded region of 4-12 nucleotides long. In some embodiments, the duplex region is 13 or 14 nucleotides long. A 6 or 7 nucleotide single stranded region is preferred in some embodiments. The single stranded region of the new RNAi compounds also comprises 2-12 phosphorothioate internucleotide linkages (referred to as phosphorothioate modifications). 6-8 phosphorothioate internucleotide linkages are preferred in some embodiments. Additionally, the RNAi compounds of the invention also include a unique chemical modification pattern, which provides stability and is compatible with RISC entry. The combination of these elements has resulted in unexpected properties which are highly useful for delivery of RNAi reagents in vitro and in vivo.

The chemical modification pattern, which provides stability and is compatible with RISC entry includes modifications to the sense, or passenger, strand as well as the antisense, or guide, strand. For instance the passenger strand can be modified with any chemical entities which confirm stability and do not interfere with activity. Such modifications include 2′ ribo modifications (O-methyl, 2′ F, 2 deoxy and others) and backbone modification like phosphorothioate modifications. A preferred chemical modification pattern in the passenger strand includes Omethyl modification of C and U nucleotides within the passenger strand or alternatively the passenger strand may be completely Omethyl modified.

The guide strand, for example, may also be modified by any chemical modification which confirms stability without interfering with RISC entry. A preferred chemical modification pattern in the guide strand includes the majority of C and U nucleotides being 2′ F modified and the 5′ end being phosphorylated. Another preferred chemical modification pattern in the guide strand includes 2′ Omethyl modification of position 1 and C/U in positions 11-18 and 5′ end chemical phosphorylation. Yet another preferred chemical modification pattern in the guide strand includes 2′ Omethyl modification of position 1 and C/U in positions 11-18 and 5′ end chemical phosphorylation and 2′F modification of C/U in positions 2-10. In some embodiments the passenger strand and/or the guide strand contains at least one 5-methyl C or U modifications.

In some embodiments, at least 30% of the nucleotides in the sd-rxRNA are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the sd-rxRNA are modified. In some embodiments, 100% of the nucleotides in the sd-rxRNA are modified.

The above-described chemical modification patterns of the oligonucleotides of the invention are well tolerated and actually improved efficacy of asymmetric RNAi compounds. In some embodiments, elimination of any of the described components (Guide strand stabilization, phosphorothioate stretch, sense strand stabilization and hydrophobic conjugate) or increase in size in some instances results in sub-optimal efficacy and in some instances complete lost of efficacy. The combination of elements results in development of a compound, which is fully active following passive delivery to cells such as HeLa cells.

The sd-rxRNA can be further improved in some instances by improving the hydrophobicity of compounds using of novel types of chemistries. For example, one chemistry is related to use of hydrophobic base modifications. Any base in any position might be modified, as long as modification results in an increase of the partition coefficient of the base. The preferred locations for modification chemistries are positions 4 and 5 of the pyrimidines. The major advantage of these positions is (a) ease of synthesis and (b) lack of interference with base-pairing and A form helix formation, which are essential for RISC complex loading and target recognition. A version of sd-rxRNA compounds where multiple deoxy Uridines are present without interfering with overall compound efficacy was used. In addition major improvement in tissue distribution and cellular uptake might be obtained by optimizing the structure of the hydrophobic conjugate. In some of the preferred embodiment the structure of sterol is modified to alter (increase/decrease) C17 attached chain. This type of modification results in significant increase in cellular uptake and improvement of tissue uptake prosperities in vivo.

dsRNA formulated according to the invention also includes rxRNAori. rxRNAori refers to a class of RNA molecules described in and incorporated by reference from PCT Publication No. WO2009/102427 (Application No. PCT/US2009/000852), filed on Feb. 11, 2009, and entitled, “MODIFIED RNAI POLYNUCLEOTIDES AND USES THEREOF” and US Patent Publication No. US 2011-0039914 entitled “MODIFIED RNAI POLYNUCLEOTIDES AND USES THEREOF.”

In some embodiments, an rxRNAori molecule comprises a double-stranded RNA (dsRNA) construct of 12-35 nucleotides in length, for inhibiting expression of a target gene, comprising: a sense strand having a 5′-end and a 3′-end, wherein the sense strand is highly modified with 2′-modified ribose sugars, and wherein 3-6 nucleotides in the central portion of the sense strand are not modified with 2′-modified ribose sugars and, an antisense strand having a 5′-end and a 3′-end, which hybridizes to the sense strand and to mRNA of the target gene, wherein the dsRNA inhibits expression of the target gene in a sequence-dependent manner.

rxRNAori can contain any of the modifications described herein. In some embodiments, at least 30% of the nucleotides in the rxRNAori are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the rxRNAori are modified. In some embodiments, 100% of the nucleotides in the sd-rxRNA are modified. In some embodiments, only the passenger strand of the rxRNAori contains modifications.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Thus, aspects of the invention relate to isolated double stranded nucleic acid molecules comprising a guide (antisense) strand and a passenger (sense) strand. As used herein, the term “double-stranded” refers to one or more nucleic acid molecules in which at least a portion of the nucleomonomers are complementary and hydrogen bond to form a double-stranded region. In some embodiments, the length of the guide strand ranges from 16-29 nucleotides long. In certain embodiments, the guide strand is 16, 17, 18, 19, 20, 21, 22, 23, 24, 25, 26, 27, 28, or 29 nucleotides long. The guide strand has complementarity to a target gene. Complementarity between the guide strand and the target gene may exist over any portion of the guide strand. Complementarity as used herein may be perfect complementarity or less than perfect complementarity as long as the guide strand is sufficiently complementary to the target that it mediates RNAi. In some embodiments complementarity refers to less than 25%, 20%, 15%, 10%, 5%, 4%, 3%, 2%, or 1% mismatch between the guide strand and the target. Perfect complementarity refers to 100% complementarity. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g. 1, 2, 3, 4, 5 or 6 nucleotides from the 3′ end of the antisense strand, are tolerated and reduce target RNA cleavage only slightly.

While not wishing to be bound by any particular theory, in some embodiments, the guide strand is at least 16 nucleotides in length and anchors the Argonaute protein in RISC. In some embodiments, when the guide strand loads into RISC it has a defined seed region and target mRNA cleavage takes place across from position 10-11 of the guide strand. In some embodiments, the 5′ end of the guide strand is or is able to be phosphorylated. The nucleic acid molecules described herein may be referred to as minimum trigger RNA.

In some embodiments, the length of the passenger strand ranges from 8-15 nucleotides long. In certain embodiments, the passenger strand is 8, 9, 10, 11, 12, 13, 14 or 15 nucleotides long. The passenger strand has complementarity to the guide strand. Complementarity between the passenger strand and the guide strand can exist over any portion of the passenger or guide strand. In some embodiments, there is 100% complementarity between the guide and passenger strands within the double stranded region of the molecule.

Aspects of the invention relate to double stranded nucleic acid molecules with minimal double stranded regions. In some embodiments the region of the molecule that is double stranded ranges from 8-15 nucleotides long. In certain embodiments, the region of the molecule that is double stranded is 8, 9, 10, 11, 12, 13, 14 or 15 nucleotides long. In certain embodiments the double stranded region is 13 or 14 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. For example the single stranded region can be 4, 5, 6, 7, 8, 9, 10, 11 or 12 nucleotides long. However, in certain embodiments, the single stranded region can also be less than 4 or greater than 12 nucleotides long. In certain embodiments, the single stranded region is at least 6 or at least 7 nucleotides long.

RNAi constructs associated with the invention can have a thermodynamic stability (ΔG) of less than −13 kkal/mol. In some embodiments, the thermodynamic stability (ΔG) is less than −20 kkal/mol. In some embodiments there is a loss of efficacy when (ΔG) goes below −21 kkal/mol. In some embodiments a (ΔG) value higher than −13 kkal/mol is compatible with aspects of the invention. Without wishing to be bound by any theory, in some embodiments a molecule with a relatively higher (ΔG) value may become active at a relatively higher concentration, while a molecule with a relatively lower (ΔG) value may become active at a relatively lower concentration. In some embodiments, the (ΔG) value may be higher than −9 kkcal/mol. The gene silencing effects mediated by the RNAi constructs associated with the invention, containing minimal double stranded regions, are unexpected because molecules of almost identical design but lower thermodynamic stability have been demonstrated to be inactive (Rana et al 2004).

Without wishing to be bound by any theory, results described herein suggest that a stretch of 8-10 bp of dsRNA or dsDNA will be structurally recognized by protein components of RISC or co-factors of RISC. Additionally, there is a free energy requirement for the triggering compound that it may be either sensed by the protein components and/or stable enough to interact with such components so that it may be loaded into the Argonaute protein. If optimal thermodynamics are present and there is a double stranded portion that is preferably at least 8 nucleotides then the duplex will be recognized and loaded into the RNAi machinery.

In some embodiments, thermodynamic stability is increased through the use of LNA bases. In some embodiments, additional chemical modifications are introduced. Several non-limiting examples of chemical modifications include: 5′ Phosphate, 2′-O-methyl, 2′-O-ethyl, 2′-fluoro, ribothymidine, C-5 propynyl-dC (pdC) and C-5 propynyl-dU (pdU); C-5 propynyl-C(pC) and C-5 propynyl-U (pU); 5-methyl C, 5-methyl U, 5-methyl dC, 5-methyl dU methoxy, (2,6-diaminopurine), 5′-Dimethoxytrityl-N4-ethyl-2′-deoxyCytidine and MGB (minor groove binder). It should be appreciated that more than one chemical modification can be combined within the same molecule.

Molecules associated with the invention are optimized for increased potency and/or reduced toxicity. For example, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. Specifically, reduction in 2′F content of a molecule is predicted to reduce toxicity of the molecule. Furthermore, the number of phosphorothioate modifications in an RNA molecule can influence the uptake of the molecule into a cell, for example the efficiency of passive uptake of the molecule into a cell. Preferred embodiments of molecules described herein have no 2′F modification and yet are characterized by equal efficacy in cellular uptake and tissue penetration. Such molecules represent a significant improvement over prior art, such as molecules described by Accell and Wolfrum, which are heavily modified with extensive use of 2′F.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contains 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

In some aspects, an optimal passenger strand is approximately 11-14 nucleotides in length. The passenger strand may contain modifications that confer increased stability. One or more nucleotides in the passenger strand can be 2′OMe modified. In some embodiments, one or more of the C and/or U nucleotides in the passenger strand is 2′OMe modified, or all of the C and U nucleotides in the passenger strand are 2′OMe modified. In certain embodiments, all of the nucleotides in the passenger strand are 2′ OMe modified. One or more of the nucleotides on the passenger strand can also be phosphate-modified such as phosphorothioate modified. The passenger strand can also contain 2′ ribo, 2′F and 2 deoxy modifications or any combination of the above. Chemical modification patterns on both the guide and passenger strand can be well tolerated and a combination of chemical modifications can lead to increased efficacy and self-delivery of RNA molecules.

Aspects of the invention relate to RNAi constructs that have extended single-stranded regions relative to double stranded regions, as compared to molecules that have been used previously for RNAi. The single stranded region of the molecules may be modified to promote cellular uptake or gene silencing. In some embodiments, phosphorothioate modification of the single stranded region influences cellular uptake and/or gene silencing. The region of the guide strand that is phosphorothioate modified can include nucleotides within both the single stranded and double stranded regions of the molecule. In some embodiments, the single stranded region includes 2-12 phosphorothioate modifications. For example, the single stranded region can include 2, 3, 4, 5, 6, 7, 8, 9, 10, 11, or 12 phosphorothioate modifications. In some instances, the single stranded region contains 6-8 phosphorothioate modifications.

Molecules associated with the invention are also optimized for cellular uptake. In RNA molecules described herein, the guide and/or passenger strands can be attached to a conjugate. In certain embodiments the conjugate is hydrophobic. The hydrophobic conjugate can be a small molecule with a partition coefficient that is higher than 10. The conjugate can be a sterol-type molecule such as cholesterol, or a molecule with an increased length polycarbon chain attached to C17, and the presence of a conjugate can influence the ability of an RNA molecule to be taken into a cell with or without a lipid transfection reagent. The conjugate can be attached to the passenger or guide strand through a hydrophobic linker. In some embodiments, a hydrophobic linker is 5-12C in length, and/or is hydroxypyrrolidine-based. In some embodiments, a hydrophobic conjugate is attached to the passenger strand and the CU residues of either the passenger and/or guide strand are modified. In some embodiments, at least 50%, 55%, 60%, 65%, 70%, 75%, 80%, 85%, 90% or 95% of the CU residues on the passenger strand and/or the guide strand are modified. In some aspects, molecules associated with the invention are self-delivering (sd). As used herein, “self-delivery” refers to the ability of a molecule to be delivered into a cell without the need for an additional delivery vehicle such as a transfection reagent.

Aspects of the invention relate to selecting molecules for use in RNAi. In some embodiments, molecules that have a double stranded region of 8-15 nucleotides can be selected for use in RNAi. In some embodiments, molecules are selected based on their thermodynamic stability (ΔG). In some embodiments, molecules will be selected that have a (ΔG) of less than −13 kkal/mol. For example, the (ΔG) value may be −13, −14, −15, −16, −17, −18, −19, −21, −22 or less than −22 kkal/mol. In other embodiments, the (ΔG) value may be higher than −13 kkal/mol. For example, the (ΔG) value may be −12, −11, −10, −9, −8, −7 or more than −7 kkal/mol. It should be appreciated that ΔG can be calculated using any method known in the art. In some embodiments ΔG is calculated using Mfold, available through the Mfold internet site (mfold.bioinfo.rpi.edu/cgi-bin/rna-form1.cgi). Methods for calculating ΔG are described in, and are incorporated by reference from, the following references: Zuker, M. (2003) Nucleic Acids Res., 31(13):3406-15; Mathews, D. H., Sabina, J., Zuker, M. and Turner, D. H. (1999) J. Mol. Biol. 288:911-940; Mathews, D. H., Disney, M. D., Childs, J. L., Schroeder, S. J., Zuker, M., and Turner, D. H. (2004) Proc. Natl. Acad. Sci. 101:7287-7292; Duan, S., Mathews, D. H., and Turner, D. H. (2006) Biochemistry 45:9819-9832; Wuchty, S., Fontana, W., Hofacker, I. L., and Schuster, P. (1999) Biopolymers 49:145-165.

In certain embodiments, the polynucleotide contains 5′- and/or 3′-end overhangs. The number and/or sequence of nucleotides overhang on one end of the polynucleotide may be the same or different from the other end of the polynucleotide. In certain embodiments, one or more of the overhang nucleotides may contain chemical modification(s), such as phosphorothioate or 2′-OMe modification.

In certain embodiments, the polynucleotide is unmodified. In other embodiments, at least one nucleotide is modified. In further embodiments, the modification includes a 2′-H or 2′-modified ribose sugar at the 2nd nucleotide from the 5′-end of the guide sequence. The “2nd nucleotide” is defined as the second nucleotide from the 5′-end of the polynucleotide.

As used herein, “2′-modified ribose sugar” includes those ribose sugars that do not have a 2′-OH group. “2′-modified ribose sugar” does not include 2′-deoxyribose (found in unmodified canonical DNA nucleotides). For example, the 2′-modified ribose sugar may be 2′-O-alkyl nucleotides, 2′-deoxy-2′-fluoro nucleotides, 2′-deoxy nucleotides, or combination thereof.

In certain embodiments, the 2′-modified nucleotides are pyrimidine nucleotides (e.g., C/U). Examples of 2′-O-alkyl nucleotides include 2′-O-methyl nucleotides, or 2′-O-allyl nucleotides.

In certain embodiments, the sd-rxRNA polynucleotide of the invention with the above-referenced 5′-end modification exhibits significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the RNAi reagent or therapeutics.

As used herein, “off-target” gene silencing refers to unintended gene silencing due to, for example, spurious sequence homology between the antisense (guide) sequence and the unintended target mRNA sequence.

According to this aspect of the invention, certain guide strand modifications further increase nuclease stability, and/or lower interferon induction, without significantly decreasing RNAi activity (or no decrease in RNAi activity at all).

In some embodiments, the 5′-stem sequence may comprise a 2′-modified ribose sugar, such as 2′-O-methyl modified nucleotide, at the 2nd nucleotide on the 5′-end of the polynucleotide and, in some embodiments, no other modified nucleotides. The hairpin structure having such modification may have enhanced target specificity or reduced off-target silencing compared to a similar construct without the 2′-O-methyl modification at said position.

Certain combinations of specific 5′-stem sequence and 3′-stem sequence modifications may result in further unexpected advantages, as partly manifested by enhanced ability to inhibit target gene expression, enhanced serum stability, and/or increased target specificity, etc.

In certain embodiments, the guide strand comprises a 2′-O-methyl modified nucleotide at the 2nd nucleotide on the 5′-end of the guide strand and no other modified nucleotides.

In other aspects, the sd-rxRNA structures of the present invention mediates sequence-dependent gene silencing by a microRNA mechanism. As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

microRNAs are involved in down-regulating target genes in critical pathways, such as development and cancer, in mice, worms and mammals. Gene silencing through a microRNA mechanism is achieved by specific yet imperfect base-pairing of the miRNA and its target messenger RNA (mRNA). Various mechanisms may be used in microRNA-mediated down-regulation of target mRNA expression.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNAs are expressed by endogenous genes in vivo and are processed from a hairpin or stem-loop precursor (pre-miRNA or pri-miRNAs) by Dicer or other RNAses. miRNAs can exist transiently in vivo as a double-stranded duplex but only one strand is taken up by the RISC complex to direct gene silencing.

In some embodiments a version of sd-rxRNA compounds, which are effective in cellular uptake and inhibiting of miRNA activity are described. Essentially the compounds are similar to RISC entering version but large strand chemical modification patterns are optimized in the way to block cleavage and act as an effective inhibitor of the RISC action. For example, the compound might be completely or mostly Omethyl modified with the PS content described previously. For these types of compounds the 5′ phosphorylation is not necessary. The presence of double stranded region is preferred as it is promotes cellular uptake and efficient RISC loading.

Another pathway that uses small RNAs as sequence-specific regulators is the RNA interference (RNAi) pathway, which is an evolutionarily conserved response to the presence of double-stranded RNA (dsRNA) in the cell. The dsRNAs are cleaved into ˜20-base pair (bp) duplexes of small-interfering RNAs (siRNAs) by Dicer. These small RNAs get assembled into multiprotein effector complexes called RNA-induced silencing complexes (RISCs). The siRNAs then guide the cleavage of target mRNAs with perfect complementarity.

Some aspects of biogenesis, protein complexes, and function are shared between the siRNA pathway and the miRNA pathway. The subject single-stranded polynucleotides may mimic the dsRNA in the siRNA mechanism, or the microRNA in the miRNA mechanism.

In certain embodiments, the modified RNAi constructs may have improved stability in serum and/or cerebral spinal fluid compared to an unmodified RNAi constructs having the same sequence.

In certain embodiments, the structure of the RNAi construct does not induce interferon response in primary cells, such as mammalian primary cells, including primary cells from human, mouse and other rodents, and other non-human mammals. In certain embodiments, the RNAi construct may also be used to inhibit expression of a target gene in an invertebrate organism.

To further increase the stability of the subject constructs in vivo, the 3′-end of the hairpin structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety.

The RNAi constructs of the invention are capable of inhibiting the synthesis of any target protein encoded by target gene(s). The invention includes methods to inhibit expression of a target gene either in a cell in vitro, or in vivo. As such, the RNAi constructs of the invention are useful for treating a patient with a disease characterized by the overexpression of a target gene.

The target gene can be endogenous or exogenous (e.g., introduced into a cell by a virus or using recombinant DNA technology) to a cell. Such methods may include introduction of RNA into a cell in an amount sufficient to inhibit expression of the target gene. By way of example, such an RNA molecule may have a guide strand that is complementary to the nucleotide sequence of the target gene, such that the composition inhibits expression of the target gene.

The invention also relates to vectors expressing the nucleic acids of the invention, and cells comprising such vectors or the nucleic acids. The cell may be a mammalian cell in vivo or in culture, such as a human cell.

The invention further relates to compositions comprising the subject RNAi constructs, and a pharmaceutically acceptable carrier or diluent.

The method may be carried out in vitro, ex vivo, or in vivo, in, for example, mammalian cells in culture, such as a human cell in culture.

The target cells (e.g., mammalian cell) may be contacted in the presence of a delivery reagent, such as a lipid (e.g., a cationic lipid) or a liposome.

Another aspect of the invention provides a method for inhibiting the expression of a target gene in a mammalian cell, comprising contacting the mammalian cell with a vector expressing the subject RNAi constructs.

In one aspect of the invention, a longer duplex polynucleotide is provided, including a first polynucleotide that ranges in size from about 16 to about 30 nucleotides; a second polynucleotide that ranges in size from about 26 to about 46 nucleotides, wherein the first polynucleotide (the antisense strand) is complementary to both the second polynucleotide (the sense strand) and a target gene, and wherein both polynucleotides form a duplex and wherein the first polynucleotide contains a single stranded region longer than 6 bases in length and is modified with alternative chemical modification pattern, and/or includes a conjugate moiety that facilitates cellular delivery. In this embodiment, between about 40% to about 90% of the nucleotides of the passenger strand between about 40% to about 90% of the nucleotides of the guide strand, and between about 40% to about 90% of the nucleotides of the single stranded region of the first polynucleotide are chemically modified nucleotides.

In an embodiment, the chemically modified nucleotide in the polynucleotide duplex may be any chemically modified nucleotide known in the art, such as those discussed in detail above. In a particular embodiment, the chemically modified nucleotide is selected from the group consisting of 2′ F modified nucleotides, 2′-O-methyl modified and 2′deoxy nucleotides. In another particular embodiment, the chemically modified nucleotides results from “hydrophobic modifications” of the nucleotide base. In another particular embodiment, the chemically modified nucleotides are phosphorothioates. In an additional particular embodiment, chemically modified nucleotides are combination of phosphorothioates, 2′-O-methyl, 2′deoxy, hydrophobic modifications and phosphorothioates. As these groups of modifications refer to modification of the ribose ring, back bone and nucleotide, it is feasible that some modified nucleotides will carry a combination of all three modification types.

In another embodiment, the chemical modification is not the same across the various regions of the duplex. In a particular embodiment, the first polynucleotide (the passenger strand), has a large number of diverse chemical modifications in various positions. For this polynucleotide up to 90% of nucleotides might be chemically modified and/or have mismatches introduced.

In another embodiment, chemical modifications of the first or second polynucleotide include, but not limited to, 5′ position modification of Uridine and Cytosine (4-pyridyl, 2-pyridyl, indolyl, phenyl (C₆H₅OH); tryptophanyl (C8H6N)CH2CH(NH2)CO), isobutyl, butyl, aminobenzyl; phenyl; naphthyl, etc), where the chemical modification might alter base pairing capabilities of a nucleotide. For the guide strand an important feature of this aspect of the invention is the position of the chemical modification relative to the 5′ end of the antisense and sequence. For example, chemical phosphorylation of the 5′ end of the guide strand is usually beneficial for efficacy. O-methyl modifications in the seed region of the sense strand (position 2-7 relative to the 5′ end) are not generally well tolerated, whereas 2′F and deoxy are well tolerated. The mid part of the guide strand and the 3′ end of the guide strand are more permissive in a type of chemical modifications applied. Deoxy modifications are not tolerated at the 3′ end of the guide strand.

A unique feature of this aspect of the invention involves the use of hydrophobic modification on the bases. In one embodiment, the hydrophobic modifications are preferably positioned near the 5′ end of the guide strand, in other embodiments, they localized in the middle of the guides strand, in other embodiment they localized at the 3′ end of the guide strand and yet in another embodiment they are distributed thought the whole length of the polynucleotide. The same type of patterns is applicable to the passenger strand of the duplex.

The other part of the molecule is a single stranded region. In some embodiments, the single stranded region is expected to range from 7 to 40 nucleotides.

In one embodiment, the single stranded region of the first polynucleotide contains modifications selected from the group consisting of between 40% and 90% hydrophobic base modifications, between 40%-90% phosphorothioates, between 40%-90% modification of the ribose moiety, and any combination of the preceding.

Efficiency of guide strand (first polynucleotide) loading into the RISC complex might be altered for heavily modified polynucleotides, so in one embodiment, the duplex polynucleotide includes a mismatch between nucleotide 9, 11, 12, 13, or 14 on the guide strand (first polynucleotide) and the opposite nucleotide on the sense strand (second polynucleotide) to promote efficient guide strand loading.

More detailed aspects of the invention are described in the sections below.

### Duplex Characteristics

Double-stranded oligonucleotides of the invention may be formed by two separate complementary nucleic acid strands. Duplex formation can occur either inside or outside the cell containing the target gene.

As used herein, the term “duplex” includes the region of the double-stranded nucleic acid molecule(s) that is (are) hydrogen bonded to a complementary sequence. Double-stranded oligonucleotides of the invention may comprise a nucleotide sequence that is sense to a target gene and a complementary sequence that is antisense to the target gene. The sense and antisense nucleotide sequences correspond to the target gene sequence, e.g., are identical or are sufficiently identical to effect target gene inhibition (e.g., are about at least about 98% identical, 96% identical, 94%, 90% identical, 85% identical, or 80% identical) to the target gene sequence.

In certain embodiments, the double-stranded oligonucleotide of the invention is double-stranded over its entire length, i.e., with no overhanging single-stranded sequence at either end of the molecule, i.e., is blunt-ended. In other embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). Likewise, when a single nucleic acid molecule is used a portion of the molecule at either end can remain single-stranded.

In one embodiment, a double-stranded oligonucleotide of the invention contains mismatches and/or loops or bulges, but is double-stranded over at least about 70% of the length of the oligonucleotide. In another embodiment, a double-stranded oligonucleotide of the invention is double-stranded over at least about 80% of the length of the oligonucleotide. In another embodiment, a double-stranded oligonucleotide of the invention is double-stranded over at least about 90%-95% of the length of the oligonucleotide. In another embodiment, a double-stranded oligonucleotide of the invention is double-stranded over at least about 96%-98% of the length of the oligonucleotide. In certain embodiments, the double-stranded oligonucleotide of the invention contains at least or up to 1, 2, 3, 4, 5, 6, 7, 8, 9, 10, 11, 12, 13, 14, or 15 mismatches.

### Modifications

The nucleotides of the invention may be modified at various locations, including the sugar moiety, the phosphodiester linkage, and/or the base.

In some embodiments, the base moiety of a nucleoside may be modified. For example, a pyrimidine base may be modified at the 2, 3, 4, 5, and/or 6 position of the pyrimidine ring. In some embodiments, the exocyclic amine of cytosine may be modified. A purine base may also be modified. For example, a purine base may be modified at the 1, 2, 3, 6, 7, or 8 position. In some embodiments, the exocyclic amine of adenine may be modified. In some cases, a nitrogen atom in a ring of a base moiety may be substituted with another atom, such as carbon. A modification to a base moiety may be any suitable modification. Examples of modifications are known to those of ordinary skill in the art. In some embodiments, the base modifications include alkylated purines or pyrimidines, acylated purines or pyrimidines, or other heterocycles.

In some embodiments, a pyrimidine may be modified at the 5 position. For example, the 5 position of a pyrimidine may be modified with an alkyl group, an alkynyl group, an alkenyl group, an acyl group, or substituted derivatives thereof. In other examples, the 5 position of a pyrimidine may be modified with a hydroxyl group or an alkoxyl group or substituted derivative thereof. Also, the N⁴ position of a pyrimidine may be alkylated. In still further examples, the pyrimidine 5-6 bond may be saturated, a nitrogen atom within the pyrimidine ring may be substituted with a carbon atom, and/or the O² and O⁴ atoms may be substituted with sulfur atoms. It should be understood that other modifications are possible as well.

In other examples, the N⁷ position and/or N² and/or N³ position of a purine may be modified with an alkyl group or substituted derivative thereof. In further examples, a third ring may be fused to the purine bicyclic ring system and/or a nitrogen atom within the purine ring system may be substituted with a carbon atom. It should be understood that other modifications are possible as well.

Non-limiting examples of pyrimidines modified at the 5 position are disclosed in U.S. Pat. No. 5,591,843, U.S. Pat. No. 7,205,297, U.S. Pat. No. 6,432,963, and U.S. Pat. No. 6,020,483; non-limiting examples of pyrimidines modified at the N⁴ position are disclosed in U.S. Pat. No. 5,580,731; non-limiting examples of purines modified at the 8 position are disclosed in U.S. Pat. No. 6,355,787 and U.S. Pat. No. 5,580,972; non-limiting examples of purines modified at the N⁶ position are disclosed in U.S. Pat. No. 4,853,386, U.S. Pat. No. 5,789,416, and U.S. Pat. No. 7,041,824; and non-limiting examples of purines modified at the 2 position are disclosed in U.S. Pat. No. 4,201,860 and U.S. Pat. No. 5,587,469, all of which are incorporated herein by reference.

Non-limiting examples of modified bases include N⁴,N⁴-ethanocytosine, 7-deazaxanthosine, 7-deazaguanosine, 8-oxo-N⁶-methyladenine, 4-acetylcytosine, 5-(carboxyhydroxylmethyl) uracil, 5-fluorouracil, 5-bromouracil, 5-carboxymethylaminomethyl-2-thiouracil, 5-carboxymethylaminomethyl uracil, dihydrouracil, inosine, N⁶-isopentenyl-adenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyl uracil, 5-methoxy aminomethyl-2-thiouracil, 5-methoxyuracil, 2-methylthio-N⁶-isopentenyladenine, pseudouracil, 5-methyl-2-thiouracil, 2-thiouracil, 4-thiouracil, 5-methyluracil, 2-thiocytosine, and 2,6-diaminopurine. In some embodiments, the base moiety may be a heterocyclic base other than a purine or pyrimidine. The heterocyclic base may be optionally modified and/or substituted.

Sugar moieties include natural, unmodified sugars, e.g., monosaccharide (such as pentose, e.g., ribose, deoxyribose), modified sugars and sugar analogs. In general, possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One particularly useful group of modified nucleomonomers are 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the invention may contain both methylated and unmethylated nucleomonomers.

Some exemplary modified nucleomonomers include sugar- or backbone-modified ribonucleotides. Modified ribonucleotides may contain a non-naturally occurring base (instead of a naturally occurring base), such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alkoxy (or OR), R or alkyl, halogen, SH, SR, amino (such as NH₂, NHR, NR₂), or CN group, wherein R is lower alkyl, alkenyl, or alkynyl.

Modified ribonucleotides may also have the phosphodiester group connecting to adjacent ribonucleotides replaced by a modified group, e.g., of phosphorothioate group. More generally, the various nucleotide modifications may be combined.

Although the antisense (guide) strand may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter antisense gene. In some cases, the antisense strand generally will be substantially identical (although in antisense orientation) to the target gene.

The use of 2′-O-methyl modified RNA may also be beneficial in circumstances in which it is desirable to minimize cellular stress responses. RNA having 2′-O-methyl nucleomonomers may not be recognized by cellular machinery that is thought to recognize unmodified RNA. The use of 2′-O-methylated or partially 2′-O-methylated RNA may avoid the interferon response to double-stranded nucleic acids, while maintaining target RNA inhibition. This may be useful, for example, for avoiding the interferon or other cellular stress responses, both in short RNAi (e.g., siRNA) sequences that induce the interferon response, and in longer RNAi sequences that may induce the interferon response.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary nucleomonomers can be found, e.g., in U.S. Pat. No. 5,849,902, incorporated by reference herein.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In certain embodiments, oligonucleotides of the invention comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-ethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceeded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “lower alkyl” is used to indicate those alkyl groups (cyclic, acyclic, substituted, unsubstituted, branched, or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments described herein.

The terms “halo” and “halogen” as used herein refer to an atom selected from fluorine, chlorine, bromine, and iodine.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻ (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function.

Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The terms “polynucleotide,” “nucleotide sequence,” “nucleic acid,” “nucleic acid molecule,” “nucleic acid sequence,” and “oligonucleotide” refer to a polymer of two or more nucleotides. The polynucleotides can be DNA, RNA, or derivatives or modified versions thereof. The polynucleotide may be single-stranded or double-stranded. The polynucleotide can be modified at the base moiety, sugar moiety, or phosphate backbone, for example, to improve stability of the molecule, its hybridization parameters, etc. The polynucleotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The olynucleotide may comprise a modified sugar moiety (e.g., 2′-fluororibose, ribose, 2′-deoxyribose, 2′-O-methylcytidine, arabinose, and hexose), and/or a modified phosphate moiety (e.g., phosphorothioates and 5′-N-phosphoramidite linkages). A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA, and RNA-RNA hybrids, as well as “protein nucleic acids” (PNA) formed by conjugating bases to an amino acid backbone.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In a preferred embodiment, the nucleomonomers of an oligonucleotide of the invention are RNA nucleotides. In another preferred embodiment, the nucleomonomers of an oligonucleotide of the invention are modified RNA nucleotides. Thus, the oligonucleotides contain modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

The nucleic acid molecules may be associated with a hydrophobic moiety for targeting and/or delivery of the molecule to a cell. In certain embodiments, the hydrophobic moiety is associated with the nucleic acid molecule through a linker. In certain embodiments, the association is through non-covalent interactions. In other embodiments, the association is through a covalent bond. Any linker known in the art may be used to associate the nucleic acid with the hydrophobic moiety. Linkers known in the art are described in published international PCT applications, WO 92/03464, WO 95/23162, WO 2008/021157, WO 2009/021157, WO 2009/134487, WO 2009/126933, U.S. Patent Application Publication 2005/0107325, U.S. Pat. No. 5,414,077, U.S. Pat. No. 5,419,966, U.S. Pat. No. 5,512,667, U.S. Pat. No. 5,646,126, and U.S. Pat. No. 5,652,359, which are incorporated herein by reference. The linker may be as simple as a covalent bond to a multi-atom linker. The linker may be cyclic or acyclic. The linker may be optionally substituted. In certain embodiments, the linker is capable of being cleaved from the nucleic acid. In certain embodiments, the linker is capable of being hydrolyzed under physiological conditions. In certain embodiments, the linker is capable of being cleaved by an enzyme (e.g., an esterase or phosphodiesterase). In certain embodiments, the linker comprises a spacer element to separate the nucleic acid from the hydrophobic moiety. The spacer element may include one to thirty carbon or heteroatoms. In certain embodiments, the linker and/or spacer element comprises protonatable functional groups. Such protonatable functional groups may promote the endosomal escape of the nucleic acid molecule. The protonatable functional groups may also aid in the delivery of the nucleic acid to a cell, for example, neutralizing the overall charge of the molecule. In other embodiments, the linker and/or spacer element is biologically inert (that is, it does not impart biological activity or function to the resulting nucleic acid molecule).

In certain embodiments, the nucleic acid molecule with a linker and hydrophobic moiety is of the formulae described herein. In certain embodiments, the nucleic acid molecule is of the formula:

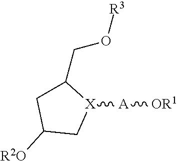

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the molecule is of the formula:

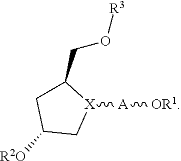

In certain embodiments, the molecule is of the formula:

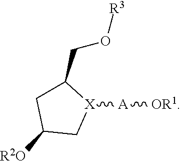

In certain embodiments, the molecule is of the formula:

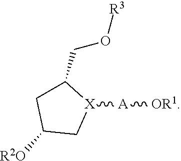

In certain embodiments, the molecule is of the formula:

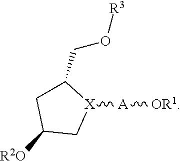

In certain embodiments, X is N. In certain embodiments, X is CH.

In certain embodiments, A is a bond. In certain embodiments, A is substituted or unsubstituted, cyclic or acyclic, branched or unbranched aliphatic. In certain embodiments, A is acyclic, substituted or unsubstituted, branched or unbranched aliphatic. In certain embodiments, A is acyclic, substituted, branched or unbranched aliphatic. In certain embodiments, A is acyclic, substituted, unbranched aliphatic. In certain embodiments, A is acyclic, substituted, unbranched alkyl. In certain embodiments, A is acyclic, substituted, unbranched C₁₋₂₀ alkyl. In certain embodiments, A is acyclic, substituted, unbranched C₁₋₁₂ alkyl. In certain embodiments, A is acyclic, substituted, unbranched C₁₁₀ alkyl. In certain embodiments, A is acyclic, substituted, unbranched C₁₋₈ alkyl. In certain embodiments, A is acyclic, substituted, unbranched C₁₋₆ alkyl. In certain embodiments, A is substituted or unsubstituted, cyclic or acyclic, branched or unbranched heteroaliphatic. In certain embodiments, A is acyclic, substituted or unsubstituted, branched or unbranched heteroaliphatic. In certain embodiments, A is acyclic, substituted, branched or unbranched heteroaliphatic. In certain embodiments, A is acyclic, substituted, unbranched heteroaliphatic.

In certain embodiments, A is of the formula:

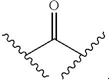 [SMILES: *C(*)=O]

In certain embodiments, A is of one of the formulae:

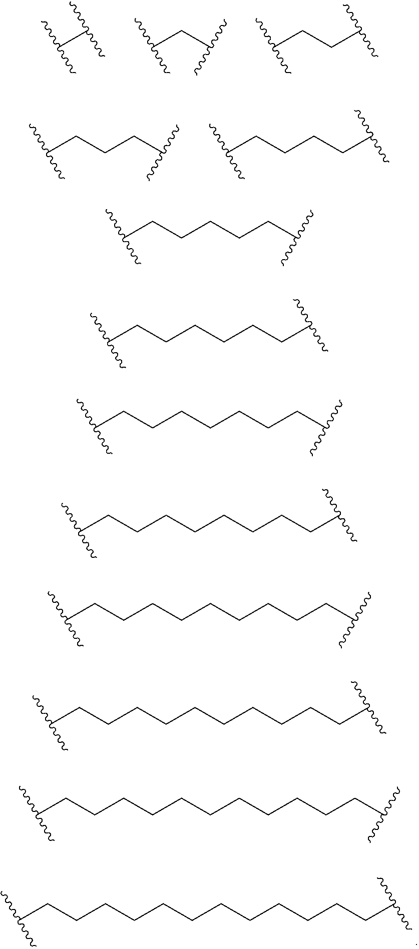

In certain embodiments, A is of one of the formulae:

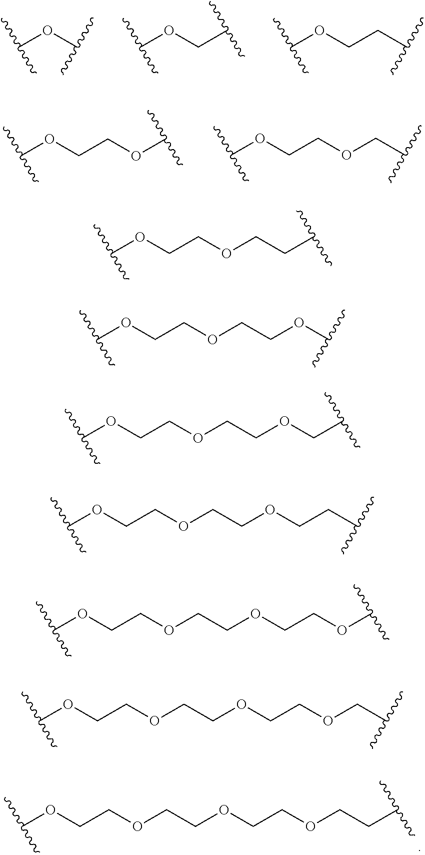

In certain embodiments, A is of one of the formulae:

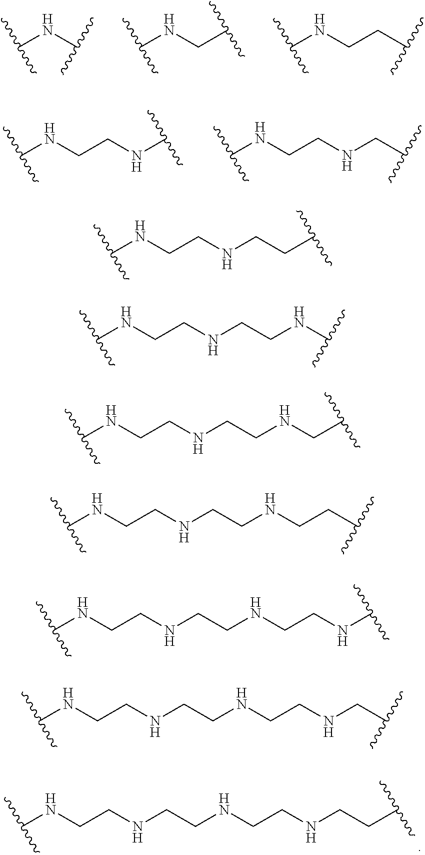

In certain embodiments, A is of the formula:

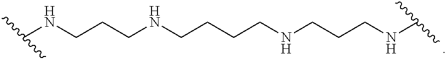 [SMILES: *NCCCNCCCCNCCCN*]

In certain embodiments, A is of the formula:

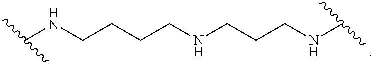 [SMILES: *NCCCCNCCCN*]

In certain embodiments, A is of the formula:

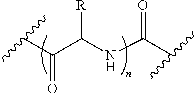

wherein

each occurrence of R is independently the side chain of a natural or unnatural amino acid; and

n is an integer between 1 and 20, inclusive. In certain embodiments, A is of the formula:

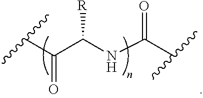

In certain embodiments, each occurrence of R is independently the side chain of a natural amino acid. In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

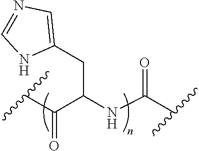

wherein n is an integer between 1 and 20, inclusive. In certain embodiments, A is of the formula:

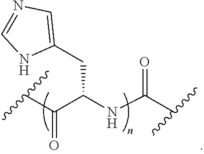

In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

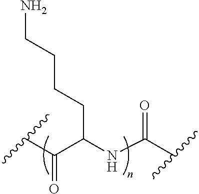

wherein n is an integer between 1 and 20, inclusive. In certain embodiments, A is of the formula:

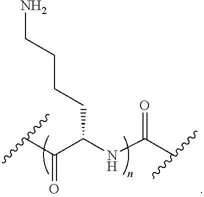

In certain embodiments, n is an integer between 1 and 15, inclusive. In certain embodiments, n is an integer between 1 and 10, inclusive. In certain embodiments, n is an integer between 1 and 5, inclusive.

In certain embodiments, the molecule is of the formula:

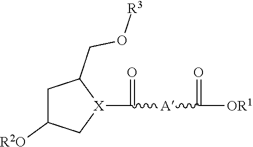

wherein X, R¹, R², and R³ are as defined herein; and

A′ is substituted or unsubstituted, cyclic or acyclic, branched or unbranched aliphatic; or substituted or unsubstituted, cyclic or acyclic, branched or unbranched heteroaliphatic.

In certain embodiments, A′ is of one of the formulae:

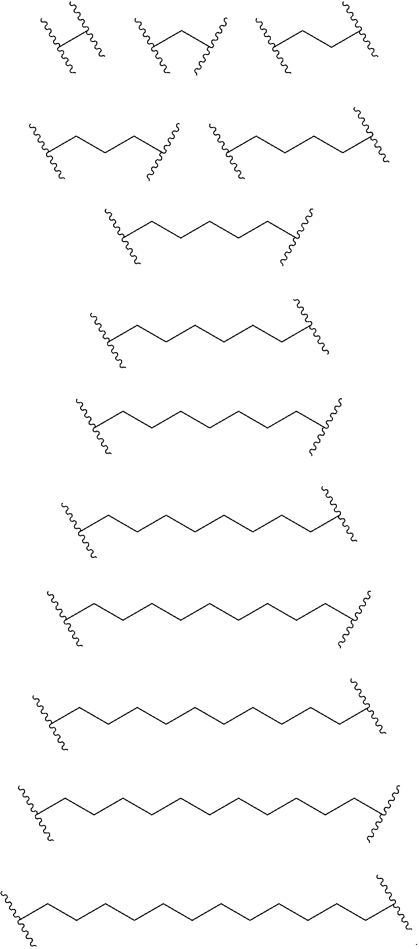

In certain embodiments, A is of one of the formulae:

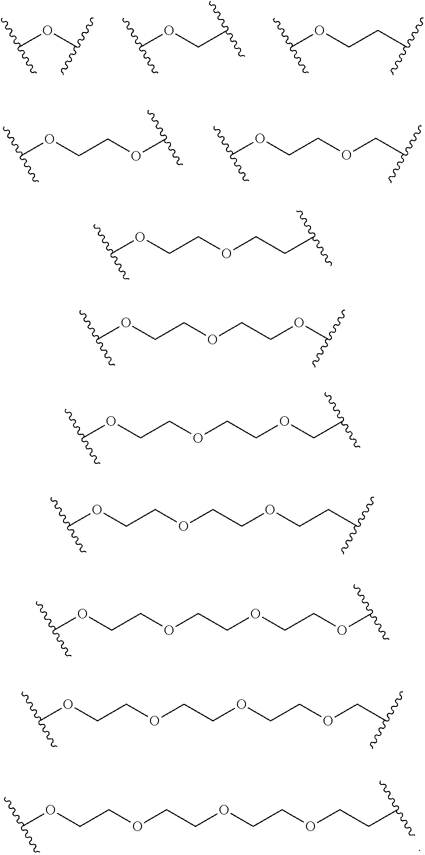

In certain embodiments, A is of one of the formulae:

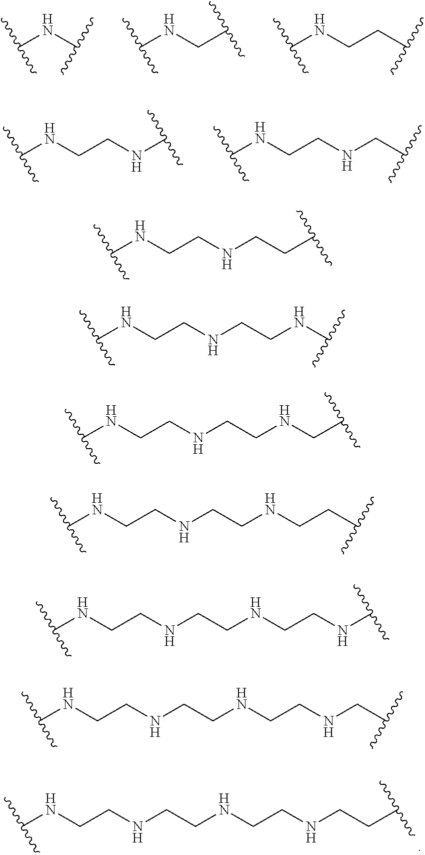

In certain embodiments, A is of the formula:

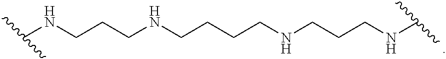 [SMILES: *NCCCNCCCCNCCCN*]

In certain embodiments, A is of the formula:

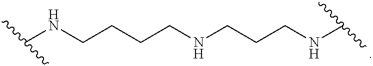 [SMILES: *NCCCCNCCCN*]

In certain embodiments, R is a steroid. In certain embodiments, R is a cholesterol. In certain embodiments, R¹ is a lipophilic vitamin. In certain embodiments, R¹ is a vitamin A. In certain embodiments, R¹ is a vitamin E.

In certain embodiments, R¹ is of the formula:

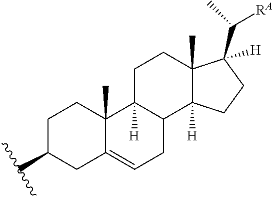

wherein R^{A} is substituted or unsubstituted, cyclic or acyclic, branched or unbranched aliphatic; or substituted or unsubstituted, cyclic or acyclic, branched or unbranched heteroaliphatic.

In certain embodiments, R¹ is of the formula:

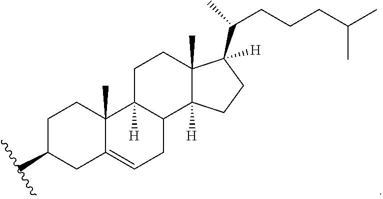

In certain embodiments, R¹ is of the formula:

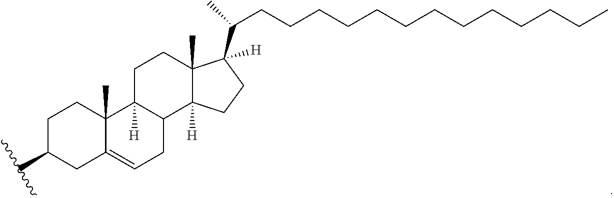

In certain embodiments, R¹ is of the formula:

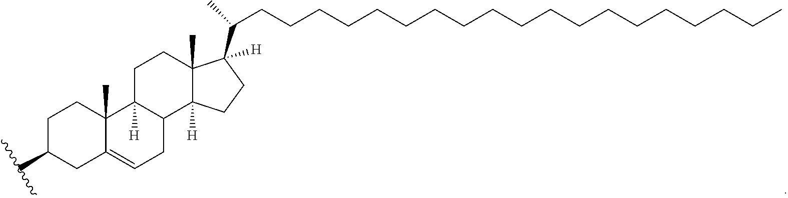

In certain embodiments, R¹ is of the formula:

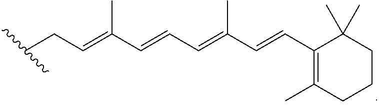 [SMILES: *C/C=C(C)/C=C/C=C(C)/C=C/C1=C(C)CCCC1(C)C]

In certain embodiments, R¹ is of the formula:

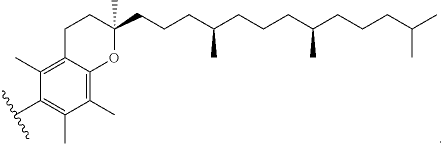 [SMILES: *c1c(C)c(C)c2c(c1C)CC[C@@](C)(CCC[C@H](C)CCC[C@H](C)CCCC(C)C)O2]

In certain embodiments, the nucleic acid molecule is of the formula:

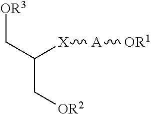

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the nucleic acid molecule is of the formula:

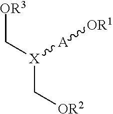

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the nucleic acid molecule is of the formula:

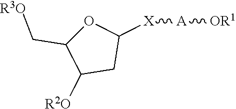

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid. In certain embodiments, the nucleic acid molecule is of the formula:

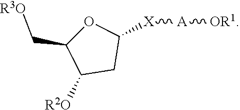

In certain embodiments, the nucleic acid molecule is of the formula:

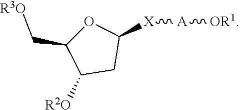

In certain embodiments, the nucleic acid molecule is of the formula:

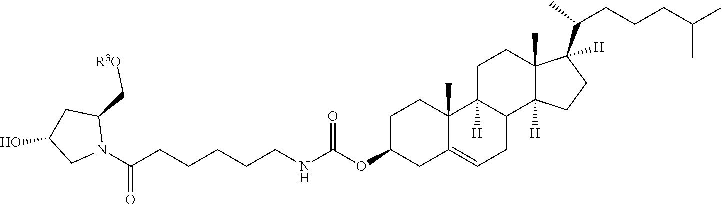

wherein R³ is a nucleic acid.

In certain embodiments, the nucleic acid molecule is of the formula:

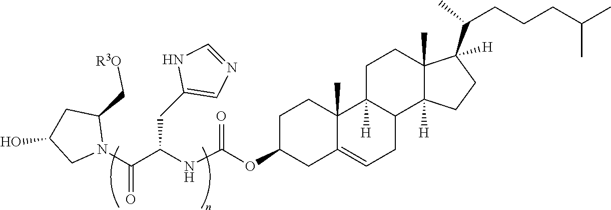

wherein R³ is a nucleic acid; and

n is an integer between 1 and 20, inclusive.

In certain embodiments, the nucleic acid molecule is of the formula:

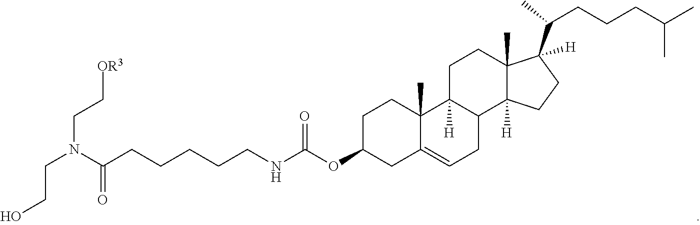

In certain embodiments, the nucleic acid molecule is of the formula:

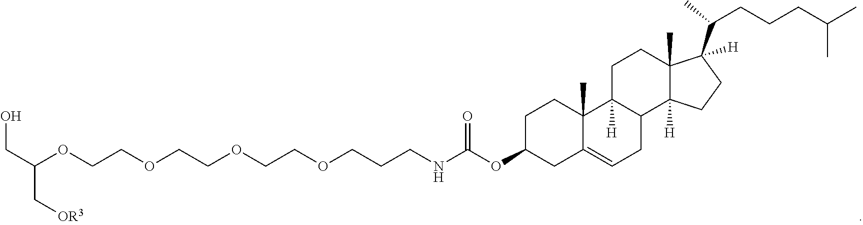

In certain embodiments, the nucleic acid molecule is of the formula:

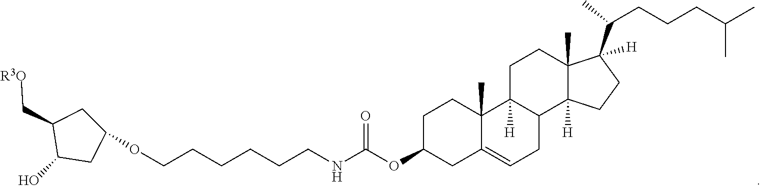

In certain embodiments, the nucleic acid molecule is of the formula:

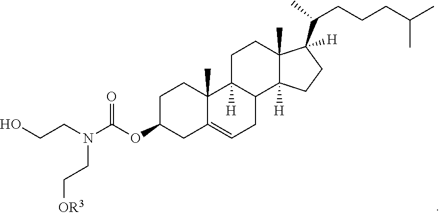

In certain embodiments, the nucleic acid molecule is of the formula:

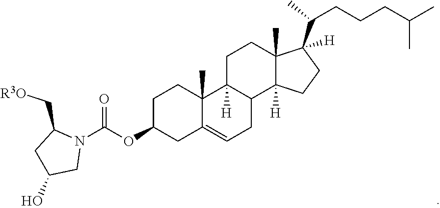

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothiate linkages.

In certain embodiments, oligonucleotides of the invention comprise hydrophobicly modified nucleotides or “hydrophobic modifications.” As used herein “hydrophobic modifications” refers to bases that are modified such that (1) overall hydrophobicity of the base is significantly increased, and/or (2) the base is still capable of forming close to regular Watson-Crick interaction. Several non-limiting examples of base modifications include 5-position uridine and cytidine modifications such as phenyl, 4-pyridyl, 2-pyridyl, indolyl, and isobutyl, phenyl (C6H5OH); tryptophanyl (C8H6N)CH2CH(NH2)CO), Isobutyl, butyl, aminobenzyl; phenyl; and naphthyl.

Another type of conjugates that can be attached to the end (3′ or 5′ end), the loop region, or any other parts of the sd-rxRNA might include a sterol, sterol type molecule, peptide, small molecule, protein, etc. In some embodiments, a sdrxRNA may contain more than one conjugates (same or different chemical nature). In some embodiments, the conjugate is cholesterol.

Another way to increase target gene specificity, or to reduce off-target silencing effect, is to introduce a 2′-modification (such as the 2′-O methyl modification) at a position corresponding to the second 5′-end nucleotide of the guide sequence. This allows the positioning of this 2′-modification in the Dicer-resistant hairpin structure, thus enabling one to design better RNAi constructs with less or no off-target silencing.

In one embodiment, a hairpin polynucleotide of the invention can comprise one nucleic acid portion which is DNA and one nucleic acid portion which is RNA. Antisense (guide) sequences of the invention can be “chimeric oligonucleotides” which comprise an RNA-like and a DNA-like region.

The language “RNase H activating region” includes a region of an oligonucleotide, e.g., a chimeric oligonucleotide, that is capable of recruiting RNase H to cleave the target RNA strand to which the oligonucleotide binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See, e.g., U.S. Pat. No. 5,849,902). Preferably, the RNase H activating region comprises about nine contiguous deoxyribose containing nucleomonomers.

The language “non-activating region” includes a region of an antisense sequence, e.g., a chimeric oligonucleotide, that does not recruit or activate RNase H. Preferably, a non-activating region does not comprise phosphorothioate DNA. The oligonucleotides of the invention comprise at least one non-activating region. In one embodiment, the non-activating region can be stabilized against nucleases or can provide specificity for the target by being complementary to the target and forming hydrogen bonds with the target nucleic acid molecule, which is to be bound by the oligonucleotide.

In one embodiment, at least a portion of the contiguous polynucleotides are linked by a substitute linkage, e.g., a phosphorothioate linkage.

In certain embodiments, most or all of the nucleotides beyond the guide sequence (2′-modified or not) are linked by phosphorothioate linkages. Such constructs tend to have improved pharmacokinetics due to their higher affinity for serum proteins. The phosphorothioate linkages in the non-guide sequence portion of the polynucleotide generally do not interfere with guide strand activity, once the latter is loaded into RISC.

Antisense (guide) sequences of the present invention may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

The chemical modifications described herein are believed, based on the data described herein, to promote single stranded polynucleotide loading into the RISC. Single stranded polynucleotides have been shown to be active in loading into RISC and inducing gene silencing. However, the level of activity for single stranded polynucleotides appears to be 2 to 4 orders of magnitude lower when compared to a duplex polynucleotide.

The present invention provides a description of the chemical modification patterns, which may (a) significantly increase stability of the single stranded polynucleotide (b) promote efficient loading of the polynucleotide into the RISC complex and (c) improve uptake of the single stranded nucleotide by the cell. The chemical modification patterns may include combination of ribose, backbone, hydrophobic nucleoside and conjugate type of modifications. In addition, in some of the embodiments, the 5′ end of the single polynucleotide may be chemically phosphorylated.

In yet another embodiment, the present invention provides a description of the chemical modifications patterns, which improve functionality of RISC inhibiting polynucleotides. Single stranded polynucleotides have been shown to inhibit activity of a preloaded RISC complex through the substrate competition mechanism. For these types of molecules, conventionally called antagomers, the activity usually requires high concentration and in vivo delivery is not very effective. The present invention provides a description of the chemical modification patterns, which may (a) significantly increase stability of the single stranded polynucleotide (b) promote efficient recognition of the polynucleotide by the RISC as a substrate and/or (c) improve uptake of the single stranded nucleotide by the cell. The chemical modification patterns may include combination of ribose, backbone, hydrophobic nucleoside and conjugate type of modifications.

The modifications provided by the present invention are applicable to all polynucleotides. This includes single stranded RISC entering polynucleotides, single stranded RISC inhibiting polynucleotides, conventional duplexed polynucleotides of variable length (15-40 bp), asymmetric duplexed polynucleotides, and the like. Polynucleotides may be modified with wide variety of chemical modification patterns, including 5′ end, ribose, backbone and hydrophobic nucleoside modifications.

### Synthesis

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; and U.S. Pat. No. 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J. Chrom. 599:35.

Other exemplary synthesis techniques are well known in the art (see, e.g., Sambrook et al., Molecular Cloning: a Laboratory Manual, Second Edition (1989); DNA Cloning, Volumes I and II (D N Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject RNAi constructs or at least portions thereof are transcribed from expression vectors encoding the subject constructs. Any art recognized vectors may be use for this purpose. The transcribed RNAi constructs may be isolated and purified, before desired modifications (such as replacing an unmodified sense strand with a modified one, etc.) are carried out.

### Delivery/Carrier

### Uptake of Oligonucleotides by Cells

Oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In a preferred embodiment, the oligonucleotide compositions of the invention are contacted with human cells.

Oligonucleotide compositions of the invention can be contacted with cells in vitro, e.g., in a test tube or culture dish, (and may or may not be introduced into a subject) or in vivo, e.g., in a subject such as a mammalian subject. In some embodiments, Oligonucleotides are administered topically or through electroporation. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet 2003 Jan. 19:9; Reichhart J M et al. Genesis. 2002. 34(1-2):1604, Yu et al. 2002. Proc. Natl. Acad Sci. USA 99:6047; Sui et al. 2002. Proc. Natl. Acad Sci. USA 99:5515) viruses, polyamine or polycation conjugates using compounds such as polylysine, protamine, or Ni, N12-bis (ethyl) spermine (see, e.g., Bartzatt, R. et al. 1989. Biotechnol. Appl. Biochem. 11:133; Wagner E. et al. 1992. Proc. Natl. Acad. Sci. 88:4255).

In certain embodiments, the sd-rxRNA of the invention may be delivered by using various beta-glucan containing particles, referred to as GeRPs (glucan encapsulated RNA loaded particle), described in, and incorporated by reference from, U.S. Provisional Application No. 61/310,611, filed on Mar. 4, 2010 and entitled “Formulations and Methods for Targeted Delivery to Phagocyte Cells.” Such particles are also described in, and incorporated by reference from US Patent Publications US 2005/0281781 A1, and US 2010/0040656, U.S. Pat. No. 8,815,818 and in PCT publications WO 2006/007372, and WO 2007/050643. The sd-rxRNA molecule may be hydrophobically modified and optionally may be associated with a lipid and/or amphiphilic peptide. In certain embodiments, the beta-glucan particle is derived from yeast. In certain embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine), etc.

Glucan particles can be derived from insoluble components of fungal cell walls such as yeast cell walls. In some embodiments, the yeast is Baker's yeast. Yeast-derived glucan molecules can include one or more of ß-(1,3)-Glucan, ß-(1,6)-Glucan, mannan and chitin. In some embodiments, a glucan particle comprises a hollow yeast cell wall whereby the particle maintains a three dimensional structure resembling a cell, within which it can complex with or encapsulate a molecule such as an RNA molecule. Some of the advantages associated with the use of yeast cell wall particles are availability of the components, their biodegradable nature, and their ability to be targeted to phagocytic cells.

In some embodiments, glucan particles can be prepared by extraction of insoluble components from cell walls, for example by extracting Baker's yeast (Fleischmann's) with IM NaOH/pH 4.0 H2O, followed by washing and drying. Methods of preparing yeast cell wall particles are discussed in, and incorporated by reference from U.S. Pat. Nos. 4,810,646, 4,992,540, 5,082,936, 5,028,703, 5,032,401, 5,322,841, 5,401,727, 5,504,079, 5,607,677, 5,968,811, 6,242,594, 6,444,448, 6,476,003, US Patent Publications 2003/0216346, 2004/0014715 and 2010/0040656, and PCT published application WO02/12348.

Protocols for preparing glucan particles are also described in, and incorporated by reference from, the following references: Soto and Ostroff (2008), “Characterization of multilayered nanoparticles encapsulated in yeast cell wall particles for DNA delivery.” Bioconjug Chem 19(4):840-8; Soto and Ostroff (2007), “Oral Macrophage Mediated Gene Delivery System,” Nanotech, Volume 2, Chapter 5 (“Drug Delivery”), pages 378-381; and Li et al. (2007), “Yeast glucan particles activate murine resident macrophages to secrete proinflammatory cytokines via MyD88- and Syk kinase-dependent pathways.” Clinical Immunology 124(2):170-181.

Glucan containing particles such as yeast cell wall particles can also be obtained commercially. Several non-limiting examples include: Nutricell MOS 55 from Biorigin (Sao Paolo, Brazil), SAF-Mannan (SAF Agri, Minneapolis, Minn.), Nutrex (Sensient Technologies, Milwaukee, Wis.), alkali-extracted particles such as those produced by Nutricepts (Nutricepts Inc., Burnsville, Minn.) and ASA Biotech, acid-extracted WGP particles from Biopolymer Engineering, and organic solvent-extracted particles such as Adjuvax™ from Alpha-beta Technology, Inc. (Worcester, Mass.) and microparticulate glucan from Novogen (Stamford, Conn.).

Glucan particles such as yeast cell wall particles can have varying levels of purity depending on the method of production and/or extraction. In some instances, particles are alkali-extracted, acid-extracted or organic solvent-extracted to remove intracellular components and/or the outer mannoprotein layer of the cell wall. Such protocols can produce particles that have a glucan (w/w) content in the range of 50%-90%. In some instances, a particle of lower purity, meaning lower glucan w/w content may be preferred, while in other embodiments, a particle of higher purity, meaning higher glucan w/w content may be preferred.

Glucan particles, such as yeast cell wall particles, can have a natural lipid content. For example, the particles can contain 1%, 2%, 3%, 4%, 5%, 6%, 7%, 8%, 9%, 10%, 11%, 12%, 13%, 14%, 15%, 16%, 17%, 18%, 19%, 20% or more than 20% w/w lipid. In the Examples section, the effectiveness of two glucan particle batches are tested: YGP SAF and YGP SAF+L (containing natural lipids). In some instances, the presence of natural lipids may assist in complexation or capture of RNA molecules.

Glucan containing particles typically have a diameter of approximately 2-4 microns, although particles with a diameter of less than 2 microns or greater than 4 microns are also compatible with aspects of the invention.

The RNA molecule(s) to be delivered are complexed or “trapped” within the shell of the glucan particle. The shell or RNA component of the particle can be labeled for visualization, as described in, and incorporated by reference from, Soto and Ostroff (2008) Bioconjug Chem 19:840. Methods of loading GeRPs are discussed further below.

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Encapsulating Agents

Encapsulating agents entrap oligonucleotides within vesicles. In another embodiment of the invention, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present invention may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some aspects, formulations associated with the invention might be selected for a class of naturally occurring or chemically synthesized or modified saturated and unsaturated fatty acid residues. Fatty acids might exist in a form of triglycerides, diglycerides or individual fatty acids. In another embodiment, the use of well-validated mixtures of fatty acids and/or fat emulsions currently used in pharmacology for parenteral nutrition may be utilized.

Liposome based formulations are widely used for oligonucleotide delivery. However, most of commercially available lipid or liposome formulations contain at least one positively charged lipid (cationic lipids). The presence of this positively charged lipid is believed to be essential for obtaining a high degree of oligonucleotide loading and for enhancing liposome fusogenic properties. Several methods have been performed and published to identify optimal positively charged lipid chemistries. However, the commercially available liposome formulations containing cationic lipids are characterized by a high level of toxicity. In vivo limited therapeutic indexes have revealed that liposome formulations containing positive charged lipids are associated with toxicity (i.e. elevation in liver enzymes) at concentrations only slightly higher than concentration required to achieve RNA silencing.

Nucleic acids associated with the invention can be hydrophobically modified and can be encompassed within neutral nanotransporters. Further description of neutral nanotransporters is incorporated by reference from PCT Application PCT/US2009/005251, filed on Sep. 22, 2009, and entitled “Neutral Nanotransporters.” Such particles enable quantitative oligonucleotide incorporation into non-charged lipid mixtures. The lack of toxic levels of cationic lipids in such neutral nanotransporter compositions is an important feature.

As demonstrated in PCT/US2009/005251, oligonucleotides can effectively be incorporated into a lipid mixture that is free of cationic lipids and such a composition can effectively deliver a therapeutic oligonucleotide to a cell in a manner that it is functional. For example, a high level of activity was observed when the fatty mixture was composed of a phosphatidylcholine base fatty acid and a sterol such as a cholesterol. For instance, one preferred formulation of neutral fatty mixture is composed of at least 20% of DOPC or DSPC and at least 20% of sterol such as cholesterol. Even as low as 1:5 lipid to oligonucleotide ratio was shown to be sufficient to get complete encapsulation of the oligonucleotide in a non charged formulation.

The neutral nanotransporters compositions enable efficient loading of oligonucleotide into neutral fat formulation. The composition includes an oligonucleotide that is modified in a manner such that the hydrophobicity of the molecule is increased (for example a hydrophobic molecule is attached (covalently or no-covalently) to a hydrophobic molecule on the oligonucleotide terminus or a non-terminal nucleotide, base, sugar, or backbone), the modified oligonucleotide being mixed with a neutral fat formulation (for example containing at least 25% of cholesterol and 25% of DOPC or analogs thereof). A cargo molecule, such as another lipid can also be included in the composition. This composition, where part of the formulation is build into the oligonucleotide itself, enables efficient encapsulation of oligonucleotide in neutral lipid particles.

In some aspects, stable particles ranging in size from 50 to 140 nm can be formed upon complexing of hydrophobic oligonucleotides with preferred formulations. It is interesting to mention that the formulation by itself typically does not form small particles, but rather, forms agglomerates, which are transformed into stable 50-120 nm particles upon addition of the hydrophobic modified oligonucleotide.

The neutral nanotransporter compositions of the invention include a hydrophobic modified polynucleotide, a neutral fatty mixture, and optionally a cargo molecule. A “hydrophobic modified polynucleotide” as used herein is a polynucleotide of the invention (i.e. sd-rxRNA) that has at least one modification that renders the polynucleotide more hydrophobic than the polynucleotide was prior to modification. The modification may be achieved by attaching (covalently or non-covalently) a hydrophobic molecule to the polynucleotide. In some instances the hydrophobic molecule is or includes a lipophilic group.

The term “lipophilic group” means a group that has a higher affinity for lipids than its affinity for water. Examples of lipophilic groups include, but are not limited to, cholesterol, a cholesteryl or modified cholesteryl residue, adamantine, dihydrotesterone, long chain alkyl, long chain alkenyl, long chain alkynyl, oleyl-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. The cholesterol moiety may be reduced (e.g. as in cholestan) or may be substituted (e.g. by halogen). A combination of different lipophilic groups in one molecule is also possible.

The hydrophobic molecule may be attached at various positions of the polynucleotide. As described above, the hydrophobic molecule may be linked to the terminal residue of the polynucleotide such as the 3′ of 5′-end of the polynucleotide. Alternatively, it may be linked to an internal nucleotide or a nucleotide on a branch of the polynucleotide. The hydrophobic molecule may be attached, for instance to a 2′-position of the nucleotide. The hydrophobic molecule may also be linked to the heterocyclic base, the sugar or the backbone of a nucleotide of the polynucleotide.

The hydrophobic molecule may be connected to the polynucleotide by a linker moiety. Optionally the linker moiety is a non-nucleotidic linker moiety. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g. via phosphodiester, phosphorothioate, methylphosphonate, or amide linkages.

Typical conjugation protocols involve the synthesis of polynucleotides bearing an aminolinker at one or more positions of the sequence, however, a linker is not required. The amino group is then reacted with the molecule being conjugated using appropriate coupling or activating reagents. The conjugation reaction may be performed either with the polynucleotide still bound to a solid support or following cleavage of the polynucleotide in solution phase. Purification of the modified polynucleotide by HPLC typically results in a pure material.

In some embodiments the hydrophobic molecule is a sterol type conjugate, a PhytoSterol conjugate, cholesterol conjugate, sterol type conjugate with altered side chain length, fatty acid conjugate, any other hydrophobic group conjugate, and/or hydrophobic modifications of the internal nucleoside, which provide sufficient hydrophobicity to be incorporated into micelles.

For purposes of the present invention, the term “sterols”, refers or steroid alcohols are a subgroup of steroids with a hydroxyl group at the 3-position of the A-ring. They are amphipathic lipids synthesized from acetyl-coenzyme A via the HMG-CoA reductase pathway. The overall molecule is quite flat. The hydroxyl group on the A ring is polar. The rest of the aliphatic chain is non-polar. Usually sterols are considered to have an 8 carbon chain at position 17.

For purposes of the present invention, the term “sterol type molecules”, refers to steroid alcohols, which are similar in structure to sterols. The main difference is the structure of the ring and number of carbons in a position 21 attached side chain.

For purposes of the present invention, the term “PhytoSterols” (also called plant sterols) are a group of steroid alcohols, phytochemicals naturally occurring in plants. There are more then 200 different known PhytoSterols

For purposes of the present invention, the term “Sterol side chain” refers to a chemical composition of a side chain attached at the position 17 of sterol-type molecule. In a standard definition sterols are limited to a 4 ring structure carrying a 8 carbon chain at position 17. In this invention, the sterol type molecules with side chain longer and shorter than conventional are described. The side chain may branched or contain double back bones.

Thus, sterols useful in the invention, for example, include cholesterols, as well as unique sterols in which position 17 has attached side chain of 2-7 or longer then 9 carbons. In a particular embodiment, the length of the polycarbon tail is varied between 5 and 9 carbons. Such conjugates may have significantly better in vivo efficacy, in particular delivery to liver. These types of molecules are expected to work at concentrations 5 to 9 fold lower then oligonucleotides conjugated to conventional cholesterols.

Alternatively the polynucleotide may be bound to a protein, peptide or positively charged chemical that functions as the hydrophobic molecule. The proteins may be selected from the group consisting of protamine, dsRNA binding domain, and arginine rich peptides. Exemplary positively charged chemicals include spermine, spermidine, cadaverine, and putrescine.

In another embodiment hydrophobic molecule conjugates may demonstrate even higher efficacy when it is combined with optimal chemical modification patterns of the polynucleotide (as described herein in detail), containing but not limited to hydrophobic modifications, phosphorothioate modifications, and 2′ ribo modifications.

In another embodiment the sterol type molecule may be a naturally occurring PhytoSterols. The polycarbon chain may be longer than 9 and may be linear, branched and/or contain double bonds. Some PhytoSterol containing polynucleotide conjugates may be significantly more potent and active in delivery of polynucleotides to various tissues. Some PhytoSterols may demonstrate tissue preference and thus be used as a way to delivery RNAi specifically to particular tissues.

The hydrophobic modified polynucleotide is mixed with a neutral fatty mixture to form a micelle. The neutral fatty acid mixture is a mixture of fats that has a net neutral or slightly net negative charge at or around physiological pH that can form a micelle with the hydrophobic modified polynucleotide. For purposes of the present invention, the term “micelle” refers to a small nanoparticle formed by a mixture of non charged fatty acids and phospholipids. The neutral fatty mixture may include cationic lipids as long as they are present in an amount that does not cause toxicity. In preferred embodiments the neutral fatty mixture is free of cationic lipids. A mixture that is free of cationic lipids is one that has less than 1% and preferably 0% of the total lipid being cationic lipid. The term “cationic lipid” includes lipids and synthetic lipids having a net positive charge at or around physiological pH. The term “anionic lipid” includes lipids and synthetic lipids having a net negative charge at or around physiological pH.

The neutral fats bind to the oligonucleotides of the invention by a strong but non-covalent attraction (e.g., an electrostatic, van der Waals, pi-stacking, etc. interaction).

The neutral fat mixture may include formulations selected from a class of naturally occurring or chemically synthesized or modified saturated and unsaturated fatty acid residues. Fatty acids might exist in a form of triglycerides, diglycerides or individual fatty acids. In another embodiment the use of well-validated mixtures of fatty acids and/or fat emulsions currently used in pharmacology for parenteral nutrition may be utilized.

The neutral fatty mixture is preferably a mixture of a choline based fatty acid and a sterol. Choline based fatty acids include for instance, synthetic phosphocholine derivatives such as DDPC, DLPC, DMPC, DPPC, DSPC, DOPC, POPC, and DEPC. DOPC (chemical registry number 4235-95-4) is dioleoylphosphatidylcholine (also known as dielaidoylphosphatidylcholine, dioleoyl-PC, dioleoylphosphocholine, dioleoyl-sn-glycero-3-phosphocholine, dioleylphosphatidylcholine). DSPC (chemical registry number 816-94-4) is distearoylphosphatidylcholine (also known as 1,2-Distearoyl-sn-Glycero-3-phosphocholine).

The sterol in the neutral fatty mixture may be for instance cholesterol. The neutral fatty mixture may be made up completely of a choline based fatty acid and a sterol or it may optionally include a cargo molecule. For instance, the neutral fatty mixture may have at least 20% or 25% fatty acid and 20% or 25% sterol.

For purposes of the present invention, the term “Fatty acids” relates to conventional description of fatty acid. They may exist as individual entities or in a form of two- and triglycerides. For purposes of the present invention, the term “fat emulsions” refers to safe fat formulations given intravenously to subjects who are unable to get enough fat in their diet. It is an emulsion of soy bean oil (or other naturally occurring oils) and egg phospholipids. Fat emulsions are being used for formulation of some insoluble anesthetics. In this disclosure, fat emulsions might be part of commercially available preparations like Intralipid, Liposyn, Nutrilipid, modified commercial preparations, where they are enriched with particular fatty acids or fully de novo-formulated combinations of fatty acids and phospholipids.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

50%-60% of the formulation can optionally be any other lipid or molecule. Such a lipid or molecule is referred to herein as a cargo lipid or cargo molecule. Cargo molecules include but are not limited to intralipid, small molecules, fusogenic peptides or lipids or other small molecules might be added to alter cellular uptake, endosomal release or tissue distribution properties. The ability to tolerate cargo molecules is important for modulation of properties of these particles, if such properties are desirable. For instance the presence of some tissue specific metabolites might drastically alter tissue distribution profiles. For example use of Intralipid type formulation enriched in shorter or longer fatty chains with various degrees of saturation affects tissue distribution profiles of these type of formulations (and their loads).

An example of a cargo lipid useful according to the invention is a fusogenic lipid. For instance, the zwitterionic lipid DOPE (chemical registry number 4004-5-1,1,2-Dioleoyl-sn-Glycero-3-phosphoethanolamine) is a preferred cargo lipid.

Intralipid may be comprised of the following composition: 1 000 mL contain: purified soybean oil 90 g, purified egg phospholipids 12 g, glycerol anhydrous 22 g, water for injection q.s. ad 1 000 mL. pH is adjusted with sodium hydroxide to pH approximately 8. Energy content/L: 4.6 MJ (190 kcal). Osmolality (approx.): 300 mOsm/kg water. In another embodiment fat emulsion is Liposyn that contains 5% safflower oil, 5% soybean oil, up to 1.2% egg phosphatides added as an emulsifier and 2.5% glycerin in water for injection. It may also contain sodium hydroxide for pH adjustment. pH 8.0 (6.0-9.0). Liposyn has an osmolarity of 276 m Osmol/liter (actual).

Variation in the identity, amounts and ratios of cargo lipids affects the cellular uptake and tissue distribution characteristics of these compounds. For example, the length of lipid tails and level of saturability will affect differential uptake to liver, lung, fat and cardiomyocytes. Addition of special hydrophobic molecules like vitamins or different forms of sterols can favor distribution to special tissues which are involved in the metabolism of particular compounds. In some embodiments, vitamin A or E is used. Complexes are formed at different oligonucleotide concentrations, with higher concentrations favoring more efficient complex formation.

In another embodiment, the fat emulsion is based on a mixture of lipids. Such lipids may include natural compounds, chemically synthesized compounds, purified fatty acids or any other lipids. In yet another embodiment the composition of fat emulsion is entirely artificial. In a particular embodiment, the fat emulsion is more then 70% linoleic acid. In yet another particular embodiment the fat emulsion is at least 1% of cardiolipin. Linoleic acid (LA) is an unsaturated omega-6 fatty acid. It is a colorless liquid made of a carboxylic acid with an 18-carbon chain and two cis double bonds.

In yet another embodiment of the present invention, the alteration of the composition of the fat emulsion is used as a way to alter tissue distribution of hydrophobicly modified polynucleotides. This methodology provides for the specific delivery of the polynucleotides to particular tissues.

In another embodiment the fat emulsions of the cargo molecule contain more then 70% of Linoleic acid (C18H32O2) and/or cardiolipin.

Fat emulsions, like intralipid have been used before as a delivery formulation for some non-water soluble drugs (such as Propofol, re-formulated as Diprivan). Unique features of the present invention include (a) the concept of combining modified polynucleotides with the hydrophobic compound(s), so it can be incorporated in the fat micelles and (b) mixing it with the fat emulsions to provide a reversible carrier. After injection into a blood stream, micelles usually bind to serum proteins, including albumin, HDL, LDL and other. This binding is reversible and eventually the fat is absorbed by cells. The polynucleotide, incorporated as a part of the micelle will then be delivered closely to the surface of the cells. After that cellular uptake might be happening though variable mechanisms, including but not limited to sterol type delivery.

### Complexing Agents

Complexing agents bind to the oligonucleotides of the invention by a strong but non-covalent attraction (e.g., an electrostatic, van der Waals, pi-stacking, etc. interaction). In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells. However, as discussed above, formulations free in cationic lipids are preferred in some embodiments.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. No. 4,235,871; U.S. Pat. Nos. 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93:3176). Cationic lipids and other complexing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. U.S.A. 93:3176). In one embodiment, a composition for delivering oligonucleotides of the invention comprises a number of arginine, lysine, histidine or ornithine residues linked to a lipophilic moiety (see e.g., U.S. Pat. No. 5,777,153).

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, a composition for delivering oligonucleotides of the invention comprises a natural or synthetic polypeptide having one or more gamma carboxyglutamic acid residues, or γ-Gla residues. These gamma carboxyglutamic acid residues may enable the polypeptide to bind to each other and to membrane surfaces. In other words, a polypeptide having a series of γ-Gla may be used as a general delivery modality that helps an RNAi construct to stick to whatever membrane to which it comes in contact. This may at least slow RNAi constructs from being cleared from the blood stream and enhance their chance of homing to the target.

The gamma carboxyglutamic acid residues may exist in natural proteins (for example, prothrombin has 10 γ-Gla residues). Alternatively, they can be introduced into the purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated/fine tuned to achieve different levels of “stickiness” of the polypeptide.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J Cell Biol. 128:919). In another embodiment, a Boc-Cys-(Npys)OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀ alkenyl chains, C₂-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see, e.g., Smith et al. Biochem J 1991.276: 417-2).

In one embodiment, oligonucleotides of the invention are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

### Targeting Agents

The delivery of oligonucleotides can also be improved by targeting the oligonucleotides to a cellular receptor. The targeting moieties can be conjugated to the oligonucleotides or attached to a carrier group (i.e., poly(L-lysine) or liposomes) linked to the oligonucleotides. This method is well suited to cells that display specific receptor-mediated endocytosis.

For instance, oligonucleotide conjugates to 6-phosphomannosylated proteins are internalized 20-fold more efficiently by cells expressing mannose 6-phosphate specific receptors than free oligonucleotides. The oligonucleotides may also be coupled to a ligand for a cellular receptor using a biodegradable linker. In another example, the delivery construct is mannosylated streptavidin which forms a tight complex with biotinylated oligonucleotides. Mannosylated streptavidin was found to increase 20-fold the internalization of biotinylated oligonucleotides. (Vlassov et al. 1994. Biochimica et Biophysica Acta 1197:95-108).

In addition specific ligands can be conjugated to the polylysine component of polylysine-based delivery systems. For example, transferrin-polylysine, adenovirus-polylysine, and influenza virus hemagglutinin HA-2 N-terminal fusogenic peptides-polylysine conjugates greatly enhance receptor-mediated DNA delivery in eucaryotic cells. Mannosylated glycoprotein conjugated to poly(L-lysine) in aveolar macrophages has been employed to enhance the cellular uptake of oligonucleotides. Liang et al. 1999. Pharmazie 54:559-566.

Because malignant cells have an increased need for essential nutrients such as folic acid and transferrin, these nutrients can be used to target oligonucleotides to cancerous cells. For example, when folic acid is linked to poly(L-lysine) enhanced oligonucleotide uptake is seen in promyelocytic leukaemia (HL-60) cells and human melanoma (M-14) cells. Ginobbi et al. 1997. Anticancer Res. 17:29. In another example, liposomes coated with maleylated bovine serum albumin, folic acid, or ferric protoporphyrin IX, show enhanced cellular uptake of oligonucleotides in murine macrophages, KB cells, and 2.2.15 human hepatoma cells. Liang et al. 1999. Pharmazie 54:559-566.

Liposomes naturally accumulate in the liver, spleen, and reticuloendothelial system (so-called, passive targeting). By coupling liposomes to various ligands such as antibodies are protein A, they can be actively targeted to specific cell populations. For example, protein A-bearing liposomes may be pretreated with H-2K specific antibodies which are targeted to the mouse major histocompatibility complex-encoded H-2K protein expressed on L cells. (Vlassov et al. 1994. Biochimica et Biophysica Acta 1197:95-108).

Other in vitro and/or in vivo delivery of RNAi reagents are known in the art, and can be used to deliver the subject RNAi constructs. See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2, just to name a few (all incorporated by reference).

### Alopecia and Therapeutic Targets

Alopecia areata is an autoimmune disease that involves the partial loss of hair on the scalp, full loss on the scalp (totalis), or full loss of hair on the body (universalis). Although the precise pathology of the disease is unknown, genetic, immunologic and environmental factors, such as viral infections, have been demonstrated to play a role in the development of alopecia areata. The growth cycle of a hair follicle occurs in three stages: anagen phase (active growth stage), catagen phase (short transition phase at the end of the anagen phase, signaling the end of the active growth phase) and telogen phase (resting phase). The hair follicle contains its own immunosuppressive microenvironment during the anagen phase which results in reduced immune stimulation due to reduced levels of major histocompatibility complex (MHC) class I molecules, termed the “hair follicle immune privilege”. In alopecia areata, the hair follicle immune privilege is impaired, leading to an autoimmune response against hair follicle autoantigens, resulting in the loss of hair.

In some aspects, the disclosure relates to methods for treating alopecia areata by targeting genes that are up-regulated in subjects having alopecia areata. Non-limiting examples of genes that are up-regulated in subjects having alopecia areata include Interleukin 2 (IL-2), Interleukin 15 (IL-15), Interleukin 12 (IL-12), Interleukin 17a (IL-17a), IFN-Gamma, CD 70, RORγt (RAR-related orphan receptor gamma), Tbet/Tbx21, ULBP3, MICA (MHC class 1 polypeptide-related sequence A), PRDX5, JAK1/JAK2, CTGF, Interleukin 2 receptor (IL-2R), Interleukin 15 receptor (IL-15R), Interleukin 12 receptor (IL-12R), CD 28, CD 27 and NKG2D. Examples of sequences encoding the above-described targets are listed in the Examples section.

Interleukin 2 (IL-2) is a type 1 cytokine, produced by T-cells in response to antigenic or mitogenic stimulation that regulates activities of lymphocytes. IL-2 mediates it effects by binding to IL-2 receptors. See Xing et al. (Nat Med. 2014 September; 20(9):1043-9. doi: 10.1038/nm.3645. Epub 2014 Aug. 17. Alopecia areata is driven by cytotoxic T lymphocytes and is reversed by JAK inhibition, herein incorporated by reference in its entirety).

Interleukin 2 receptor (IL-2R) is a protein expressed on lymphocytes that binds to the IL-2 cytokine.

Interleukin 15 (IL-15) is a cytokine that stimulates cell activation and proliferation of T-cells. IL-15 mediates it effects by binding to IL-15 receptors.

Interleukin 15 receptor (IL-15R) is a type 1 cytokine receptor, composed of three subunits: IL-15Rα, IL-2Rβ and IL-2Rγ.

Interleukin 12 (IL-12) is a cytokine produced by dendritic cells, macrophages and B-lymphoblastoid cells in response to antigenic stimulation. IL-12 is involved in the differentiation of naïve T-cells to Th1 cells. IL-12 is a heterodimeric protein composed of IL-12α and IL-12β.

Interleukin 12 receptor (IL-12R) is a type 1 cytokine receptor that specifically binds the IL-12 cytokine. IL-12R is composed of two subunits, IL-12Rβ1 and IL-12Rβ2.

Interleukin 17a (IL-17a): a proinflammatory cytokine produced by activated T-cells.

IFN-Gamma is a type 2 interferon, critical for innate and adaptive immunity against viral, bacterial and protozoan infections.

CD 28: is a signaling receptor on T-cells that serves as the receptor for CD80 and CD86 proteins.

CD 70 is a cytokine of the tumor necrosis family ligand family, a ligand for CD27.

CD 27 is a member of the tumor necrosis receptor family that binds to the CD70 ligand. This receptor is required for generation and maintenance of T-cell immunity.

RORγt (RAR-related orphan receptor gamma) is a transcription factor belonging to the nuclear receptor family. RORgT is involved in lymphoid organogenesis and also promotes thymocyte differentiation into Th17 cells.

Tbet/Tbx21 is a transcription factor involved in initiating the differentiation of Th1 cells from precursor cells. Tbx21 is Th1 cell-specific and controls the expression of IFN-gamma.

ULBP3 encodes a ligand for the NKG2D receptor and activates several signaling pathways in natural killer cells through binding to its receptor, NKG2D.

MICA encodes the MHC class 1 polypeptide-related sequence A, a protein that functions as a stress-induced antigen that is recognized by natural killer cells, natural killer T-cells as well as other T-cell subtypes.

NKG2D is a receptor on natural killer and CD8 T-cells. Ligands include but are not limited to MICA and ULBP3. See Petukhova et al. (Nature. 2010 Jul. 1; 466(7302):113-7. doi: 10.1038/nature09114. Genome-wide association study in alopecia areata implicates both innate and adaptive immunity, herein incorporated by reference in its entirety).

PRDX5 (peroxiredoxin 5) is a member of the peroxiredoxin family, which serve as an antioxidant in normal and inflammatory phase by reducing hydrogen peroxide and alkyl hydroperoxides.

JAK1/JAK2 encode protein-tyrosine kinases of the janus kinase family. JAK1 is essential for initiating responses to major cytokine receptor families. JAK2 is required for the IFN-gamma response. (Nat Med. 2014 September; 20(9):1043-9. doi: 10.1038/nm.3645. Epub 2014 Aug. 17. Alopecia areata is driven by cytotoxic T lymphocytes and is reversed by JAK inhibition, herein incorporated by reference in its entirety.)

CTGF (connective tissue growth factor) is a member of the CCN family of extracellular matrix-associated heparin-binding proteins and plays a role in cell adhesion, migration, proliferation, tissue wound repair and plays a key role in fibrosis.

Aspects of the invention relate to dsRNA directed against CTGF. For example, the antisense strand of a dsRNA directed against CTGF can be complementary to at least 12 contiguous nucleotides of a sequence selected from the sequences within Tables 11, 12 and 15, incorporated by reference from PCT Publication No. WO 2011/119887 and US Patent Publication No. US2014/0113950. The sense strand and/or the antisense strand of a dsRNA directed against CTGF can comprises at least 12 contiguous nucleotides of a sequence selected from the sequences within Tables 10, 11, 12, 15, 20 and 24, incorporated by reference from PCT Publication No. WO 2011/119887 and US Patent Publication No. US2014/0113950.

In some embodiments, the sense strand comprises at least 12 contiguous nucleotides of a sequence selected from the group consisting of: SEQ ID NOs: 25, 27, 30, 32, 34, 36, 38, 27 and 40 (corresponding to SEQ ID NOs: 2463, 3429, 2443, 3445, 2459, 3493, 2465, 3475 and 3469, incorporated by reference from PCT Publication No. WO 2011/119887 and US Patent Publication No. US2014/0113950). In certain embodiments, the sense strand comprises or consists of a sequence selected from the group consisting of: SEQ ID NOs: 25, 27, 30, 32, 34, 36, 38, 27 and 40 (corresponding to SEQ ID NOs: 2463, 3429, 2443, 3445, 2459, 3493, 2465, 3475 and 3469, incorporated by reference from PCT Publication No. WO 2011/119887 and US Patent Publication No. US2014/0113950).

In some embodiments, the antisense strand comprises at least 12 contiguous nucleotides of a sequence selected from the group consisting of: SEQ ID NOs: 26, 28, 31, 33, 35, 37, 39, 41 and 29 (corresponding to SEQ ID NOs: 2464, 3430, 4203, 3446, 2460, 3494, 2466, 3476 and 3470, incorporated by reference from PCT Publication No. WO 2011/119887 and US Patent Publication No. US2014/0113950). In certain embodiments, the antisense strand comprises or consists of a sequence selected from the group consisting of: SEQ ID NOs: 26, 28, 31, 33, 35, 37, 39, 41 and 29 (corresponding to SEQ ID NOs: 2464, 3430, 4203, 3446, 2460, 3494, 2466, 3476 and 3470, incorporated by reference from PCT Publication No. WO 2011/119887 and US Patent Publication No. US2014/0113950).

In a preferred embodiment, the sense strand comprises SEQ ID NO:25 (GCACCUUUCUAGA) and the antisense strand comprises SEQ ID NO:26 (UCUAGAAAGGUGCAAACAU), corresponding to and incorporated by reference from SEQ ID NOs 2463 and 2464 in PCT Publication No. WO 2011/119887 and US Patent Publication No. US2014/0113950). The sequences of SEQ ID NO:25 and SEQ ID NO:26 can be modified in a variety of ways according to modifications described herein. A preferred modification pattern for SEQ ID NO:25 is depicted by SEQ ID NO:27 (G.mC.A.mC.mC.mU.mU.mU.mC.mU. A*mG*mA.TEG-Chl), incorporated by reference from SEQ ID NOs: 3429 and 3475 in PCT Publication No. WO 2011/119887 and US Patent Publication No. US2014/0113950. A preferred modification pattern for SEQ ID NO:26 is depicted by SEQ ID NO:28 (P.mU.fC.fU. A. G.mA. A.mA. G. G.fU. G.mC* A* A* A*mC* A* U), incorporated by reference from SEQ ID NO: 3430 in PCT Publication No. WO 2011/119887 and US Patent Publication No. US2014/0113950. An sd-rxRNA consisting of a sense strand depicted by SEQ ID NO:27 (G.mC. A.mC.mC.mU.mU.mU.mC.mU. A*mG*mA.TEG-Chl) and an antisense strand depicted by SEQ ID NO:28 (P.mU.fC.fU. A. G.mA. A.mA. G. G.fU. G.mC* A* A* A*mC* A* U) is also referred to as RXI-109, as described in and incorporated by reference from SEQ ID NOs: 3429 and 3475 and SEQ ID NO: 3430 in PCT Publication No. WO 2011/119887 and US Patent Publication No. US2014/0113950. TEG-Chl refers to cholesterol with a TEG linker; m refers to 2′Ome; f refers to 2′fluoro; * refers to phosphorothioate linkage; and . refers to phosphodiester linkage.

In another preferred embodiment, the sense strand comprises SEQ ID NO:30 (UUGCACCUUUCUAA) and the antisense strand comprises SEQ ID NO:31 (UUAGAAAGGUGCAAACAAGG), incorporated by reference from SEQ ID NOs: 2443 and 4203 in PCT Publication No. WO 2011/119887 and US Patent Publication No. US2014/0113950. The sequences of SEQ ID NO:30 and SEQ ID NO:31 can be modified in a variety of ways according to modifications described herein. A preferred modification pattern for SEQ ID NO:30 is depicted by SEQ ID NO:32 (mU.mU. G.mC. A.mC.mC.mU.mU.mU.mC.mU*mA*mA.TEG-Chl), incorporated by reference from SEQ ID NO: 3445 in PCT Publication No. WO 2011/119887 and US Patent Publication No. US2014/0113950. A preferred modification pattern for SEQ ID NO:31 is depicted by SEQ ID NO:33 (P.mU.fU. A. G. A.mA. A. G. G.fU. G.fC.mA.mA*mA*fC*mA*mA*mG* G.), incorporated by reference from SEQ ID NO: 3446 in PCT Publication No. WO 2011/119887 and US Patent Publication No. US2014/0113950.

In another preferred embodiment, the sense strand comprises SEQ ID NO:34 (GUGACCAAAAGUA) and the antisense strand comprises SEQ ID NO:35 (UACUUUUGGUCACACUCUC), incorporated by reference from SEQ ID NOs 2459 and 2460 in PCT Publication No. WO 2011/119887 and US Patent Publication No. US2014/0113950. The sequences of SEQ ID NO:34 and SEQ ID NO:35 can be modified in a variety of ways according to modifications described herein. A preferred modification pattern for SEQ ID NO:34 is depicted by SEQ ID NO:36 (G.mU. G. A.mC.mC. A. A. A. A. G*mU*mA.TEG-Chl), incorporated by reference from SEQ ID NO: 3493 in PCT Publication No. WO 2011/119887 and US Patent Publication No. US2014/0113950. A preferred modification pattern for SEQ ID NO:35 is depicted by SEQ ID NO:37 (P.mU. A.fC.fU.fU.fU.fU. G. G.fU.mC. A.mC* A*mC*mU*mC*mU* C.), incorporated by reference from SEQ ID NO: 3494 in PCT Publication No. WO 2011/119887 and US Patent Publication No. US2014/0113950.

In another preferred embodiment, the sense strand comprises SEQ ID NO:38 (CCUUUCUAGUUGA) and the antisense strand comprises SEQ ID NO:39 (UCAACUAGAAAGGUGCAAA), incorporated by reference from SEQ ID NOs: 2465 and 2466 in PCT Publication No. WO 2011/119887 and US Patent Publication No. US2014/0113950. The sequences of SEQ ID NO:38 and SEQ ID NO:39 can be modified in a variety of ways according to modifications described herein. A preferred modification pattern for SEQ ID NO:38 is depicted by SEQ ID NO:40 (mC.mC.mU.mU.mU.mC.mU. A. G.mU.mU*mG*mA.TEG-Chl), incorporated by reference from SEQ ID NO: 3469 in PCT Publication No. WO 2011/119887 and US Patent Publication No. US2014/0113950. A preferred modification pattern for SEQ ID NO:39 is depicted by SEQ ID NO:29 (P.mU.fC. A. A.fC.fU. A. G. A.mA. A. G. G*fU*mG*fC*mA*mA* A.), incorporated by reference from SEQ ID NO: 3470 in PCT Publication No. WO 2011/119887 and US Patent Publication No. US2014/0113950.

In another preferred embodiment, the sense strand comprises SEQ ID NO:27 (G.mC. A.mC.mC.mU.mU.mU.mC.mU. A*mG*mA.TEG-Chl) and the antisense strand comprises SEQ ID NO:41 (P.mU.fC.fU. A. G.mA. A.mA. G. G.fU. G.fC*mA*mA*mA*fC*mA* U.) incorporated by reference from SEQ ID NOs 3475 and 3476 in PCT Publication No. WO 2011/119887 and US Patent Publication No. US2014/0113950.

### Administration

The present disclosure provides methods for treating alopecia areata by administering a hapten that elicits a T-cell response. Without wishing to be bound by any theory, the immune response induced in a subject by administering a hapten, such as DPCP, may include cellular immune responses mediated by CD8+ T-cells capable of killing tumor and infected cells, and CD4+ T-cell responses. Humoral immune responses, mediated primarily by antibody-producing B-cells may also be induced.

In some aspects, the disclosure relates to the administration of a therapeutically effective amount of a hapten to a subject in need thereof for the treatment of alopecia areata. In some embodiments, the hapten is administered to the subject by topical administration. In some embodiments, the hapten is administered to the subject more than once. In some embodiments, the hapten is administered twice, the first administration as a sensitization dose and the second administration as a challenge dose. In some embodiments, the sensitization dose (for example, in the range of about 0.1% DPCP to about 1% DPCP) is administered approximately 2 weeks prior to challenge dose. In some embodiments, the challenge dose (for example, in the range of about 0.0000001% to about 0.4% DPCP) is administered approximately two weeks post sensitization dose and then at a time period selected from the group consisting of twice a week, once every week, once every two weeks and once every three weeks, until the hair is fully regrown. In case of a relapse, dosing can be re-initiated.

In some embodiments, the disclosure provides methods for sensitizing a subject to a therapeutic modality by administering an initial sensitizing dose of hapten to a subject followed by a subsequent administration of challenge dose of hapten to the subject. Thus, in some embodiments, to enhance an immune response in a subject, the hapten is administered to the skin of a subject in an initial sensitizing dose (which elicits sensitivity to subsequent treatment) and one or more subsequent challenge dose(s).

In some embodiments, the disclosure provides a method for the treatment of alopecia areata in a subject, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the alopecia areata has been treated.

In any of these embodiments, the sensitization dose of the hapten can range from 0.1% to 1% hapten, including approximately 0.1%, 0.2%, 0.3%, 0.4%, 0.5%, 0.6%, 0.7%, 0.8%, 0.9%, and 1.0% hapten. In certain particular embodiments, the sensitization dose of the hapten is 0.4% or about 0.4% hapten. In any of these embodiments, the challenge dose of the hapten can range from 0.0000001% to 0.4% hapten (any integer between and including 0.0000001 and 0.4). In any of these embodiments, the hapten can be selected from DPCP, imiquimod, ingenol mebutate, and SADBE. In certain particular embodiments, the hapten is DPCP.

In any of these embodiments, the sensitization dose of hapten can be administered to the skin two weeks or approximately two weeks prior to the administration of the first challenge dose of hapten. In any of these embodiments, the first challenge dose can be administered to the skin subsequent to the sensitizing dose. In some embodiments, the first challenge dose is administered to the skin two weeks or about two weeks after the sensitizing dose. In some embodiments, the first challenge dose is administered to the skin earlier or later than two weeks after the sensitizing dose. For example, the first challenge dose can be administered from about 1-25 days following the initial sensitization dose, including 1, 2, 3, 4, 5, 6, 7, 8, 9, 10, 11, 12, 13, 14, 15, 16, 17, 18, 19, 20, 21, 22, 23, 24, or 25 days following the sensitization dose. In any of these embodiments, the first challenge dose of hapten can be administered to the skin following the sensitization dose and then subsequently administered on a schedule selected from 1-5 times daily, twice a day, once a day, every other day, twice a week, once a week, once every two weeks, once every three weeks, once a month, once every two months or longer, and in any schedule combination thereof until the skin disorder or condition is treated (e.g., alopecia areata). In the case of a relapse or insufficient or incomplete therapeutic effect, dosing can be re-initiated.

In any of these embodiments, the first challenge dose and the subsequent continuing challenge doses can be the same dose. In other embodiments, the first challenge dose and the subsequent continuing challenge doses can be different doses. In any of the embodiments disclosed herein, the sensitizing dose and the challenge dose(s) of hapten can be administered to the same site on the skin. In any of the embodiments described herein, the sensitizing dose and the challenge dose(s) can be administered to different sites on the skin. For example, the sensitizing dose may be applied to a normal skin area and the challenge dose may be applied to affected skin. It should be appreciated that dosing of a hapten could be optimized by one of ordinary skill in the art without undue experimentation.

In any of these embodiments, the hapten can be formulated in any of the compositions discussed herein, including gel or ointment formulations. In some embodiments, the composition comprises a non-ionic surfactant selected from polyoxyethylene (20) monoleate, polyoxyethylene (20) sorbitan monooleate, polysorbate 80, palmitate and stearate; an alcoholic ester selected from isopropyl myristate and isopropyl palmitate; and a gelling agent that is polyoxyl 40 stearate. In certain particular embodiments, the composition comprises a hapten, polysorbate 80, isopropyl myristate, and polyoxyl 40 stearate. In one particular embodiment, the composition is a formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some embodiments, a hapten, such as DPCP, is formulated as a gel comprising a) a first co-solvent comprising a non-ionic surfactant, b) a second co-solvent comprising an alcoholic ester, and c) a gelling agent. The first co-solvent can be selected from the group consisting of polyoxyethylene (20) monoleate, polyoxyethylene (20) sorbitan monooleate, palmitate and stearate, wherein the second co-solvent can be selected from the group consisting of isopropyl myristate and isopropyl palmitate, and wherein said gelling agent is polyoxyl 40 stearate.

Alternatively, the gel can be comprised of a) a first co-solvent comprising a non-ionic surfactant, b) a second co-solvent comprising an alcoholic ester, c) an alcohol and d) a thickening agent. The first co-solvent can be selected from the group consisting of polyoxyethylene (20) monoleate, polyoxyethylene (20) sorbitan monooleate, polysorbate 80 (PS80), palmitate and stearate, wherein the second co-solvent can be selected from the group consisting of isopropyl myristate and isopropyl palmitate, wherein the alcohol can be selected from the group consisting of ethanol or isopropanol and wherein the gelling agent is hydroxypropyl cellulose (Klucel™).

In other embodiments, the hapten, such as DPCP, is formulated as an ointment. The ointment can comprise a) a first co-solvent comprising a non-ionic surfactant, b) a second co-solvent comprising an alcoholic ester, and c) a thickening agent. The first co-solvent can be selected from the group consisting of polyoxyethylene (20) monoleate, polyoxyethylene (20) sorbitan monooleate, palmitate and stearate, wherein the second co-solvent can be selected from the group consisting of isopropyl myristate and isopropyl palmitate, and wherein the thickening agent can be selected from the group of and/or any combination of white wax, cetyl ester wax and/or glyceryl monostearate.

In other embodiments, the hapten, such as DPCP, is formulated as a cream, lotion, foam, patch or paste.

Hapten compositions may be applied to the skin by dabbing a cotton-tipped swab that has been saturated with solution onto the skin at the desired site of application, without repeated rubbing or spreading of the solution over an extended area. For both the sensitization and treatment applications, the hapten composition is preferably left on the skin for a period of time before washing it off. In some embodiments, the hapten composition is left on the skin for a time period selected from about 1-72 hours, about 2-60 hours, about 3-48 hours, about 4-36 hours, and about 8-24 hours.

In some aspects, the disclosure relates to the administration of a therapeutically effective amount of a nucleic acid molecule to a subject in need thereof for the treatment of alopecia areata. In some embodiments, the nucleic acid molecule is an oligonucleotide. The optimal course of administration or delivery of the oligonucleotide(s) may vary depending upon the desired result and/or on the subject to be treated. As used herein “administration” refers to contacting cells with oligonucleotides and can be performed in vitro or in vivo. The dosage of oligonucleotides may be adjusted to optimally reduce expression of a protein translated from a target nucleic acid molecule, e.g., as measured by a readout of RNA stability or by a therapeutic response, without undue experimentation.

For example, expression of the protein encoded by the nucleic acid target can be measured to determine whether or not the dosage regimen needs to be adjusted accordingly. In addition, an increase or decrease in RNA or protein levels in a cell or produced by a cell can be measured using any art recognized technique. By determining whether transcription has been decreased, the effectiveness of the oligonucleotide in inducing the cleavage of a target RNA can be determined.

Any of the compositions can be used alone or in conjunction with a pharmaceutically acceptable carrier. As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types.

With respect to in vivo applications, the formulations of the present invention can be administered to a patient in a variety of forms adapted to the chosen route of administration, e.g., parenterally, orally, or intraperitoneally. Parenteral administration, which is preferred in some embodiments, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. In preferred embodiments, the sd-rxRNA molecules are administered by intradermal injection or subcutaneously.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

Pharmaceutical preparations for topical administration include transdermal patches, ointments, lotions, creams, gels, drops, sprays, suppositories, liquids and powders. In addition, conventional pharmaceutical carriers, aqueous, powder or oily bases, or thickeners may be used in pharmaceutical preparations for topical administration.

Pharmaceutical preparations for oral administration include powders or granules, suspensions or solutions in water or non-aqueous media, capsules, sachets or tablets. In addition, thickeners, flavoring agents, diluents, emulsifiers, dispersing aids, or binders may be used in pharmaceutical preparations for oral administration.

For transmucosal or transdermal administration, penetrants appropriate to the barrier to be permeated are used in the formulation. Such penetrants are known in the art, and include, for example, for transmucosal administration bile salts and fusidic acid derivatives, and detergents. Transmucosal administration may be through nasal sprays or using suppositories. For oral administration, the oligonucleotides are formulated into conventional oral administration forms such as capsules, tablets, and tonics. For topical administration, the oligonucleotides of the invention are formulated into ointments, salves, gels, or creams as known in the art.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described oligonucleotides may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the oligonucleotide to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the oligonucleotide at the lymph node. The oligonucleotide can be modified to diffuse into the cell, or the liposome can directly participate in the delivery of either the unmodified or modified oligonucleotide into the cell.

The chosen method of delivery will result in entry into cells. In some embodiments, preferred delivery methods include liposomes (10-400 nm), hydrogels, controlled-release polymers, and other pharmaceutically applicable vehicles, and microinjection or electroporation (for ex vivo treatments).

The pharmaceutical preparations of the present invention may be prepared and formulated as emulsions. Emulsions are usually heterogeneous systems of one liquid dispersed in another in the form of droplets usually exceeding 0.1 μm in diameter. The emulsions of the present invention may contain excipients such as emulsifiers, stabilizers, dyes, fats, oils, waxes, fatty acids, fatty alcohols, fatty esters, humectants, hydrophilic colloids, preservatives, and anti-oxidants may also be present in emulsions as needed. These excipients may be present as a solution in either the aqueous phase, oily phase or itself as a separate phase.

Examples of naturally occurring emulsifiers that may be used in emulsion formulations of the present invention include lanolin, beeswax, phosphatides, lecithin and acacia. Finely divided solids have also been used as good emulsifiers especially in combination with surfactants and in viscous preparations. Examples of finely divided solids that may be used as emulsifiers include polar inorganic solids, such as heavy metal hydroxides, nonswelling clays such as bentonite, attapulgite, hectorite, kaolin, montrnorillonite, colloidal aluminum silicate and colloidal magnesium aluminum silicate, pigments and nonpolar solids such as carbon or glyceryl tristearate.

Examples of preservatives that may be included in the emulsion formulations include methyl paraben, propyl paraben, quaternary ammonium salts, benzalkonium chloride, esters of p-hydroxybenzoic acid, and boric acid. Examples of antioxidants that may be included in the emulsion formulations include free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of oligonucleotides are formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both oil/water and water/oil) have been proposed to enhance the oral bioavailability of drugs.

Microemulsions offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11:1385; Ho et al., J. Pharm. Sci., 1996, 85:138-143). Microemulsions have also been effective in the transdermal delivery of active components in both cosmetic and pharmaceutical applications. It is expected that the microemulsion compositions and formulations of the present invention will facilitate the increased systemic absorption of oligonucleotides from the gastrointestinal tract, as well as improve the local cellular uptake of oligonucleotides within the gastrointestinal tract, vagina, buccal cavity and other areas of administration.

In an embodiment, the present invention employs various penetration enhancers to affect the efficient delivery of nucleic acids, particularly oligonucleotides, to the skin of animals. Even non-lipophilic drugs may cross cell membranes if the membrane to be crossed is treated with a penetration enhancer. In addition to increasing the diffusion of non-lipophilic drugs across cell membranes, penetration enhancers also act to enhance the permeability of lipophilic drugs.

Five categories of penetration enhancers that may be used in the present invention include: surfactants, fatty acids, bile salts, chelating agents, and non-chelating non-surfactants. Other agents may be utilized to enhance the penetration of the administered oligonucleotides include: glycols such as ethylene glycol and propylene glycol, pyrrols such as 2-15 pyrrol, azones, and terpenes such as limonene, and menthone.

The oligonucleotides, especially in lipid formulations, can also be administered by coating a medical device, for example, a catheter, such as an angioplasty balloon catheter, with a cationic lipid formulation. Coating may be achieved, for example, by dipping the medical device into a lipid formulation or a mixture of a lipid formulation and a suitable solvent, for example, an aqueous-based buffer, an aqueous solvent, ethanol, methylene chloride, chloroform and the like. An amount of the formulation will naturally adhere to the surface of the device which is subsequently administered to a patient, as appropriate. Alternatively, a lyophilized mixture of a lipid formulation may be specifically bound to the surface of the device. Such binding techniques are described, for example, in K. Ishihara et al., Journal of Biomedical Materials Research, Vol. 27, pp. 1309-1314 (1993), the disclosures of which are incorporated herein by reference in their entirety.

The useful dosage to be administered and the particular mode of administration will vary depending upon such factors as the cell type, or for in vivo use, the age, weight and the particular animal and region thereof to be treated, the particular oligonucleotide and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligonucleotides, the amount of lipid compound that is administered can vary and generally depends upon the amount of oligonucleotide agent being administered. For example, the weight ratio of lipid compound to oligonucleotide agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligonucleotide agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the oligonucleotide is administered in a form in which any toxic effects are outweighed by the therapeutic effects of the oligonucleotide. In one embodiment, oligonucleotides can be administered to subjects. Examples of subjects include mammals, e.g., humans and other primates; cows, pigs, horses, and farming (agricultural) animals; dogs, cats, and other domesticated pets; mice, rats, and transgenic non-human animals.

Administration of an active amount of an oligonucleotide of the present invention is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically-modified oligonucleotides, e.g., with modification of the phosphate backbone, may require different dosing.

The exact dosage of an oligonucleotide and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regimens may be adjusted to provide the optimum therapeutic response. For example, the oligonucleotide may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

Administration of sd-rxRNAs, such as through intradermal injection or subcutaneous delivery, can be optimized through testing of dosing regimens. In some embodiments, a single administration is sufficient. To further prolong the effect of the administered sd-rxRNA, the sd-rxRNA can be administered in a slow-release formulation or device, as would be familiar to one of ordinary skill in the art. The hydrophobic nature of sd-rxRNA compounds can enable use of a wide variety of polymers, some of which are not compatible with conventional oligonucleotide delivery.

In other embodiments, the sd-rxRNA is administered multiple times. In some instances it is administered daily, bi-weekly, weekly, every two weeks, every three weeks, monthly, every two months, every three months, every four months, every five months, every six months or less frequently than every six months. In some instances, it is administered multiple times per day, week, month and/or year. For example, it can be administered approximately every hour, 2 hours, 3 hours, 4 hours, 5 hours, 6 hours, 7 hours, 8 hours, 9 hours 10 hours, 12 hours or more than twelve hours. It can be administered 1, 2, 3, 4, 5, 6, 7, 8, 9, 10 or more than 10 times per day.

Aspects of the invention relate to administering sd-rxRNA molecules to a subject. In some instances the subject is a patient and administering the sd-rxRNA molecule involves administering the sd-rxRNA molecule in a doctor's office.

In some embodiments, more than one sd-rxRNA molecule is administered simultaneously. For example a composition may be administered that contains 1, 2, 3, 4, 5, 6, 7, 8, 9, 10 or more than 10 different sd-rxRNA molecules. In certain embodiments, a composition comprises 2 or 3 different sd-rxRNA molecules. When a composition comprises more than one sd-rxRNA, the sd-rxRNA molecules within the composition can be directed to the same gene or to different genes.

In some instances, the effective amount of sd-rxRNA that is delivered by subcutaneous administration is at least approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 mg/kg including any intermediate values.

In some instances, the effective amount of sd-rxRNA that is delivered through intradermal injection is at least approximately 1, 5, 10, 15, 20, 25, 30, 35, 40, 45, 50, 55, 60, 65, 70, 75, 80, 85, 90, 95, 100, 125, 150, 200, 250, 300, 350, 400, 450, 500, 550, 600, 650, 700, 750, 800, 850, 900, 950 or more than 950 μg including any intermediate values.

sd-rxRNA molecules administered through methods described herein are effectively targeted to all the cell types in the skin.

Physical methods of introducing nucleic acids include injection of a solution containing the nucleic acid, bombardment by particles covered by the nucleic acid, soaking the cell or organism in a solution of the nucleic acid, or electroporation of cell membranes in the presence of the nucleic acid. A viral construct packaged into a viral particle would accomplish both efficient introduction of an expression construct into the cell and transcription of nucleic acid encoded by the expression construct. Other methods known in the art for introducing nucleic acids to cells may be used, such as lipid-mediated carrier transport, chemical-mediated transport, such as calcium phosphate, and the like. Thus the nucleic acid may be introduced along with components that perform one or more of the following activities: enhance nucleic acid uptake by the cell, inhibit annealing of single strands, stabilize the single strands, or other-wise increase inhibition of the target gene.

Nucleic acid may be directly introduced into the cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals.

Alternatively, vectors, e.g., transgenes encoding a siRNA of the invention can be engineered into a host cell or transgenic animal using art recognized techniques.

A further preferred use for the agents of the present invention (or vectors or transgenes encoding same) is a functional analysis to be carried out in eukaryotic cells, or eukaryotic non-human organisms, preferably mammalian cells or organisms and most preferably human cells, e.g. cell lines such as HeLa or 293 or rodents, e.g. rats and mice. By administering a suitable priming agent/RNAi agent which is sufficiently complementary to a target mRNA sequence to direct target-specific RNA interference, a specific knockout or knockdown phenotype can be obtained in a target cell, e.g. in cell culture or in a target organism.

Thus, a further subject matter of the invention is a eukaryotic cell or a eukaryotic non-human organism exhibiting a target gene-specific knockout or knockdown phenotype comprising a fully or at least partially deficient expression of at least one endogenous target gene wherein said cell or organism is transfected with at least one vector comprising DNA encoding an RNAi agent capable of inhibiting the expression of the target gene. It should be noted that the present invention allows a target-specific knockout or knockdown of several different endogenous genes due to the specificity of the RNAi agent.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

### Therapeutic Use

By inhibiting the expression of a gene, the hapten compositions and/or the oligonucleotide compositions of the present invention can be used to treat alopecia areata.

In one embodiment, in vitro treatment of cells with haptens and/or oligonucleotides can be used for ex vivo therapy of cells removed from a subject or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to evaluate gene function, to study gene regulation and protein synthesis or to evaluate improvements made to oligonucleotides designed to modulate gene expression or protein synthesis. In vivo treatment of cells can be useful in certain clinical settings where it is desirable to inhibit the expression of a protein. There are numerous medical conditions for which antisense therapy is reported to be suitable (see, e.g., U.S. Pat. No. 5,830,653) as well as respiratory syncytial virus infection (WO 95/22,553) influenza virus (WO 94/23,028), and malignancies (WO 94/08,003). Other examples of clinical uses of antisense sequences are reviewed, e.g., in Glaser. 1996. Genetic Engineering News 16:1. Exemplary targets for cleavage by oligonucleotides include, e.g., protein kinase Ca, ICAM-1, c-raf kinase, p53, c-myb, and the bcr/abl fusion gene found in chronic myelogenous leukemia.

The subject nucleic acids can be used in RNAi-based therapy in any animal having RNAi pathway, such as human, non-human primate, non-human mammal, non-human vertebrates, rodents (mice, rats, hamsters, rabbits, etc.), domestic livestock animals, pets (cats, dogs, etc.), Xenopus, fish, insects (Drosophila, etc.), and worms (C. elegans), etc.

The invention provides methods for preventing in a subject, a disease or condition associated with an aberrant or unwanted target gene expression or activity, by administering to the subject a therapeutic agent (e.g., a RNAi agent or vector or transgene encoding same). If appropriate, subjects are first treated with a priming agent so as to be more responsive to the subsequent RNAi therapy. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject.

In another aspect, the invention pertains to methods of modulating target gene expression, protein expression or activity for therapeutic purposes. Accordingly, in an exemplary embodiment, the modulatory method of the invention involves contacting a cell capable of expressing target gene with a therapeutic agent of the invention that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent), in vivo (e.g., by administering the agent to a subject), or ex vivo. Typically, subjects are first treated with a priming agent so as to be more responsive to the subsequent RNAi therapy. As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

The therapeutic agents of the invention can be administered to individuals to treat (prophylactically or therapeutically) disorders associated with aberrant or unwanted target gene activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent. Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Identification of sd-rxRNAs Useful for Treatment of Alopecia

Genes upregulated in subjects having alopecia, optionally alopecia areata, are identified by gene expression analysis. Subjects having alopecia are administered a hapten and post-treatment gene expression analysis is performed to identify genes that are suppressed by hapten treatment. Genes suppressed by the hapten treatment are then investigated as targets for sd-rxRNA.

sd-rxRNAs targeting genes associated with alopecia (for example, Interleukin 2 (IL-2), Interleukin 15 (IL-15), Interleukin 12 (IL-12), Interleukin 17a (IL-17a), IFN-Gamma, CD 70, RORγt (RAR-related orphan receptor gamma), Tbet/Tbx21, ULBP3, MICA (MHC class 1 polypeptide-related sequence A), PRDX5, JAK1/JAK2, CTGF, Interleukin 2 receptor (IL-2R), Interleukin 15 receptor (IL-15R), Interleukin 12 receptor (IL-12R), CD 28, CD 27 and NKG2D) are designed, synthesized and screened in vitro to determine the ability of the sd-rxRNAs to reduce target gene mRNA levels. The sd-rxRNAs are tested for activity in HT-1080 cells (human fibrosarcoma cell line, 10,000 cells/well, 96 well plate). HT-1080 cells are treated with varying concentrations of a panel of alopecia-associated gene-targeting sd-rxRNAs or non-targeting control (NTC) in serum-free media. Concentrations tested include 1 and 0.1 μM. The non-targeting control sd-rxRNA (NTC) is of similar structure to the alopecia-associated gene-targeting sd-rxRNA and contains similar stabilizing modifications throughout both strands. Forty-eight hours post administration, cells are lysed and mRNA levels determined by the Quantigene branched DNA assay according to the manufacture's protocol using gene-specific probes (Affymetrix). Data are normalized to a house keeping gene (PPIB) and graphed with respect to the non-targeting control.

### Example 2: Dose Response Analysis of sd-rxRNAs in HT-1080 Cells

Alopecia-associated gene-targeting sd-rxRNAs (for example, sd-RXRNAs targeting Interleukin 2 (IL-2), Interleukin 15 (IL-15), Interleukin 12 (IL-12), Interleukin 17a (IL-17a), IFN-Gamma, CD 70, RORγt (RAR-related orphan receptor gamma), Tbet/Tbx21, ULBP3, MICA (MHC class 1 polypeptide-related sequence A), PRDX5, JAK1/JAK2, CTGF, Interleukin 2 receptor (IL-2R), Interleukin 15 receptor (IL-15R), Interleukin 12 receptor (IL-12R), CD 28, CD 27 and NKG2D) are tested in an in vitro dose response study. The sd-rxRNAs are tested for activity in HT-1080 cells (human fibrosarcoma cell line, 10,000 cells/well, 96 well plate). HT-1080 cells are treated with varying concentrations of alopecia-associated gene-targeting sd-rxRNAs or non-targeting control (NTC) in serum-free media. Concentrations tested include 1, 0.5, 0.1, 0.05, 0.025 and 0.01 μM. The non-targeting control sd-rxRNA (NTC) is of similar structure to the alopecia-associated gene-targeting sd-rxRNA and contains similar stabilizing modifications throughout both strands. Forty-eight hours post administration, cells are lysed and mRNA levels determined by the Quantigene branched DNA assay according to the manufacture's protocol using gene-specific probes (Affymetrix). Data are normalized to a house keeping gene (PPIB) and graphed with respect to the non-targeting control.

Sequences of Target Genes

IL2: The human IL-2 sequence is represented by GenBank accession number
NM_000586.3 (SEQ ID NO: 1) listed below:
agttccctat cactctcttt aatcactact cacagtaacc tcaactcctg ccacaatgta
caggatgcaa ctcctgtctt gcattgcact aagtcttgca cttgtcacaa acagtgcacc
tacttcaagt tctacaaaga aaacacagct acaactggag catttactgc tggatttaca
gatgattttg aatggaatta ataattacaa gaatcccaaa ctcaccagga tgctcacatt
taagttttac atgcccaaga aggccacaga actgaaacat cttcagtgtc tagaagaaga
actcaaacct ctggaggaag tgctaaattt agctcaaagc aaaaactttc acttaagacc
cagggactta atcagcaata tcaacgtaat agttctggaa ctaaagggat ctgaaacaac
attcatgtgt gaatatgctg atgagacagc aaccattgta gaatttctga acagatggat
taccttttgt caaagcatca tctcaacact gacttgataa ttaagtgctt cccacttaaa
acatatcagg ccttctattt atttaaatat ttaaatttta tatttattgt tgaatgtatg
gtttgctacc tattgtaact attattctta atcttaaaac tataaatatg gatcttttat
gattcttttt gtaagcccta ggggctctaa aatggtttca cttatttatc ccaaaatatt
tattattatg ttgaatgtta aatatagtat ctatgtagat tggttagtaa aactatttaa
taaatttgat aaatataaaa aaaaaaaaaa aaaaaaaaaa aa
IL-2Rα: The human IL-2Rα sequence is represented by GenBank accession number
NM_000417.2 (SEQ ID NO: 2) listed below:
ggcagtttcc tggctgaaca cgccagccca atacttaaag agagcaactc ctgactccga
tagagactgg atggacccac aagggtgaca gcccaggcgg accgatcttc ccatcccaca
tcctccggcg cgatgccaaa aagaggctga cggcaactgg gccttctgca gagaaagacc
tccgcttcac tgccccggct ggtcccaagg gtcaggaaga tggattcata cctgctgatg
tggggactgc tcacgttcat catggtgcct ggctgccagg cagagctctg tgacgatgac
ccgccagaga tcccacacgc cacattcaaa gccatggcct acaaggaagg aaccatgttg
aactgtgaat gcaagagagg tttccgcaga ataaaaagcg ggtcactcta tatgctctgt
acaggaaact ctagccactc gtcctgggac aaccaatgtc aatgcacaag ctctgccact
cggaacacaa cgaaacaagt gacacctcaa cctgaagaac agaaagaaag gaaaaccaca
gaaatgcaaa gtccaatgca gccagtggac caagcgagcc ttccaggtca ctgcagggaa
cctccaccat gggaaaatga agccacagag agaatttatc atttcgtggt ggggcagatg
gtttattatc agtgcgtcca gggatacagg gctctacaca gaggtcctgc tgagagcgtc
tgcaaaatga cccacgggaa gacaaggtgg acccagcccc agctcatatg cacaggtgaa
atggagacca gtcagtttcc aggtgaagag aagcctcagg caagccccga aggccgtcct
gagagtgaga cttcctgcct cgtcacaaca acagattttc aaatacagac agaaatggct
gcaaccatgg agacgtccat atttacaaca gagtaccagg tagcagtggc cggctgtgtt
ttcctgctga tcagcgtcct cctcctgagt gggctcacct ggcagcggag acagaggaag
agtagaagaa caatctagaa aaccaaaaga acaagaattt cttggtaaga agccgggaac
agacaacaga agtcatgaag cccaagtgaa atcaaaggtg ctaaatggtc gcccaggaga
catccgttgt gcttgcctgc gttttggaag ctctgaagtc acatcacagg acacggggca
gtggcaacct tgtctctatg ccagctcagt cccatcagag agcgagcgct acccacttct
aaatagcaat ttcgccgttg aagaggaagg gcaaaaccac tagaactctc catcttattt
tcatgtatat gtgttcatta aagcatgaat ggtatggaac tctctccacc ctatatgtag
tataaagaaa agtaggttta cattcatctc attccaactt cccagttcag gagtcccaag
gaaagcccca gcactaacgt aaatacacaa cacacacact ctaccctata caactggaca
ttgtctgcgt ggttcctttc tcagccgctt ctgactgctg attctcccgt tcacgttgcc
taataaacat ccttcaagaa ctctgggctg ctacccagaa atcattttac ccttggctca
atcctctaag ctaaccccct tctactgagc cttcagtctt gaatttctaa aaaacagagg
ccatggcaga ataatctttg ggtaacttca aaacggggca gccaaaccca tgaggcaatg
tcaggaacag aaggatgaat gaggtcccag gcagagaatc atacttagca aagttttacc
tgtgcgttac taattggcct ctttaagagt tagtttcttt gggattgcta tgaatgatac
cctgaatttg gcctgcacta atttgatgtt tacaggtgga cacacaaggt gcaaatcaat
gcgtacgttt cctgagaagt gtctaaaaac accaaaaagg gatccgtaca ttcaatgttt
atgcaaggaa ggaaagaaag aaggaagtga agagggagaa gggatggagg tcacactggt
agaacgtaac cacggaaaag agcgcatcag gcctggcacg gtggctcagg cctataaccc
cagctcccta ggagaccaag gcgggagcat ctcttgaggc caggagtttg agaccagcct
gggcagcata gcaagacaca tccctacaaa aaattagaaa ttggctggat gtggtggcat
acgcctgtag tcctagccac tcaggaggct gaggcaggag gattgcttga gcccaggagt
tcgaggctgc agtcagtcat gatggcacca ctgcactcca gcctgggcaa cagagcaaga
tcctgtcttt aaggaaaaaa agacaagatg agcataccag cagtccttga acattatcaa
aaagttcagc atattagaat caccgggagg ccttgttaaa agagttcgct gggcccatct
tcagagtctc tgagttgttg gtctggaata gagccaaatg ttttgtgtgt ctaacaattc
ccaggtgctg ttgctgctgc tactattcca ggaacacact ttgagaacca ttgtgttatt
gctctgcacg cccacccact ctcaactccc acgaaaaaaa tcaacttcca gagctaagat
ttcggtggaa gtcctggttc catatctggt gcaagatctc ccctcacgaa tcagttgagt
caacattcta gctcaacaac atcacacgat taacattaac gaaaattatt catttgggaa
actatcagcc agttttcact tctgaagggg caggagagtg ttatgagaaa tcacggcagt
tttcagcagg gtccagattc agattaaata actattttct gtcatttctg tgaccaacca
catacaaaca gactcatctg tgcactctcc ccctccccct tcaggtatat gttttctgag
taaagttgaa aagaatctca gaccagaaaa tatagatata tatttaaatc ttacttgagt
agaactgatt acgacttttg ggtgttgagg ggtctataag atcaaaactt ttccatgata
atactaagat gttatcgacc atttatctgt ccttctctca aaagtgtatg gtggaatttt
ccagaagcta tgtgatacgt gatgatgtca tcactctgct gttaacatat aataaattta
ttgctattgt ttataaaaga ataaatgata tttttt
IL-21Rβ: The human IL-2Rβ sequence is represented by GenBank accession number
NM_000878.3 (SEQ ID NO: 3) listed below:
gcagccagag ctcagcaggg ccctggagag atggccacgg tcccagcacc ggggaggact
ggagagcgcg cgctgccacc gccccatgtc tcagccaggg cttccttcct cggctccacc
ctgtggatgt aatggcggcc cctgctctgt cctggcgtct gcccctcctc atcctcctcc
tgcccctggc tacctcttgg gcatctgcag cggtgaatgg cacttcccag ttcacatgct
tctacaactc gagagccaac atctcctgtg tctggagcca agatggggct ctgcaggaca
cttcctgcca agtccatgcc tggccggaca gacggcggtg gaaccaaacc tgtgagctgc
tccccgtgag tcaagcatcc tgggcctgca acctgatcct cggagcccca gattctcaga
aactgaccac agttgacatc gtcaccctga gggtgctgtg ccgtgagggg gtgcgatgga
gggtgatggc catccaggac ttcaagccct ttgagaacct tcgcctgatg gcccccatct
ccctccaagt tgtccacgtg gagacccaca gatgcaacat aagctgggaa atctcccaag
cctcccacta ctttgaaaga cacctggagt tcgaggcccg gacgctgtcc ccaggccaca
cctgggagga ggcccccctg ctgactctca agcagaagca ggaatggatc tgcctggaga
cgctcacccc agacacccag tatgagtttc aggtgcgggt caagcctctg caaggcgagt
tcacgacctg gagcccctgg agccagcccc tggccttcag gacaaagcct gcagcccttg
ggaaggacac cattccgtgg ctcggccacc tcctcgtggg cctcagcggg gcttttggct
tcatcatctt agtgtacttg ctgatcaact gcaggaacac cgggccatgg ctgaagaagg
tcctgaagtg taacacccca gacccctcga agttcttttc ccagctgagc tcagagcatg
gaggagacgt ccagaagtgg ctctcttcgc ccttcccctc atcgtccttc agccctggcg
gcctggcacc tgagatctcg ccactagaag tgctggagag ggacaaggtg acgcagctgc
tcctgcagca ggacaaggtg cctgagcccg catccttaag cagcaaccac tcgctgacca
gctgcttcac caaccagggt tacttcttct tccacctccc ggatgccttg gagatagagg
cctgccaggt gtactttact tacgacccct actcagagga agaccctgat gagggtgtgg
ccggggcacc cacagggtct tccccccaac ccctgcagcc tctgtcaggg gaggacgacg
cctactgcac cttcccctcc agggatgacc tgctgctctt ctcccccagt ctcctcggtg
gccccagccc cccaagcact gcccctgggg gcagtggggc cggtgaagag aggatgcccc
cttctttgca agaaagagtc cccagagact gggaccccca gcccctgggg cctcccaccc
caggagtccc agacctggtg gattttcagc caccccctga gctggtgctg cgagaggctg
gggaggaggt ccctgacgct ggccccaggg agggagtcag tttcccctgg tccaggcctc
ctgggcaggg ggagttcagg gcccttaatg ctcgcctgcc cctgaacact gatgcctact
tgtccctcca agaactccag ggtcaggacc caactcactt ggtgtagaca gatggccagg
gtgggaggca ggcagctgcc tgctctgcgc cgagcctcag aaggaccctg ttgagggtcc
tcagtccact gctgaggaca ctcagtgtcc agttgcagct ggacttctcc acccggatgg
cccccaccca gtcctgcaca cttggtccat ccatttccaa acctccactg ctgctcccgg
gtcctgctgc ccgagccagg aactgtgtgt gttgcagggg ggcagtaact ccccaactcc
ctcgttaatc acaggatccc acgaatttag gctcagaagc atcgctcctc tccagccctg
cagctattca ccaatatcag tcctcgcggc tctccagggc tccctgccct gacctcttcc
ctgggttttc tgccccagcc tcctccttcc ctcccctccc cgtccacagg gcagcctgag
cgtgctttcc aaaacccaaa tatggccacg ctccccctcg gttcaaaacc ttgcacaggt
cccactgccc tcagccccac ttctcagcct ggtacttgta cctccggtgt cgtgtgggga
catccccttc tgcaatcctc cctaccgtcc tcctgagcca ctcagagctc cctcacaccc
cctctgttgc acatgctatt ccctggggct gctgtgcgct ccccctcatc taggtgacaa
acttccctga ctcttcaagt gccggttttg cttctcctgg agggaagcac tgcctccctt
aatctgccag aaacttctag cgtcagtgct ggagggagaa gctgtcaggg acccagggcg
cctggagaaa gaggccctgt tactattcct ttgggatctc tgaggcctca gagtgcttgg
ctgctgtatc tttaatgctg gggcccaagt aagggcacag atccccccac aaagtggatg
cctgctgcat cttcccacag tggcttcaca gacccacaag agaagctgat ggggagtaaa
ccctggagtc cgaggcccag gcagcagccc cgcctagtgg tgggccctga tgctgccagg
cctgggacct cccactgccc cctccactgg aggggtctcc tctgcagctc agggactggc
acactggcct ccagaagggc agctccacag ggcagggcct cattattttt cactgcccca
gacacagtgc ccaacacccc gtcgtatacc ctggatgaac gaattaatta cctggcacca
cctcgtctgg gctccctgcg cctgacattc acacagagag gcagagtccc gtgcccatta
ggtctggcat gccccctcct gcaaggggct caacccccta ccccgacccc tccacgtatc
tttcctaggc agatcacgtt gcaatggctc aaacaacatt ccaccccagc aggacagtga
ccccagtccc agctaactct gacctgggag ccctcaggca cctgcactta caggccttgc
tcacagctga ttgggcacct gaccacacgc ccccacaggc tctgaccagc agcctatgag
ggggtttggc accaagctct gtccaatcag gtaggctggg cctgaactag ccaatcagat
caactctgtc ttgggcgttt gaactcaggg agggaggccc ttgggagcag gtgcttgtgg
acaaggctcc acaagcgttg agccttggaa aggtagacaa gcgttgagcc actaagcaga
ggaccttggg ttcccaatac aaaaatacct actgctgaga gggctgctga ccatttggtc
aggattcctg ttgcctttat atccaaaata aactcccctt tcttgaggtt gtctgagtct
tgggtctatg ccttgaaaaa agctgaatta ttggacagtc tcacctcctg ccatagggtc
ctgaatgttt cagaccacaa ggggctccac acctttgctg tgtgttctgg ggcaacctac
taatcctctc tgcaagtcgg tctccttatc cccccaaatg gaaattgtat ttgccttctc
cactttggga ggctcccact tcttgggagg gttacatttt ttaagtctta atcatttgtg
acatatgtat ctatacatcc gtatctttta atgatccgtg tgtaccatct ttgtgattat
ttccttaata ttttttcttt aagtcagttc attttcgttg aaatacattt atttaaagaa
aaatctttgt tactctgtaa atgaaaaaac ccattttcgc tataaataaa aggtaactgt
acaaaataag tacaatgcaa caaaaaaaaa
IL-2Rγ: The human IL-2Rγ sequence is represented by GenBank accession number
NM_000206.2 (SEQ ID NO: 4) listed below:
agaggaaacg tgtgggtggg gaggggtagt gggtgaggga cccaggttcc tgacacagac
agactacacc cagggaatga agagcaagcg ccatgttgaa gccatcatta ccattcacat
ccctcttatt cctgcagctg cccctgctgg gagtggggct gaacacgaca attctgacgc
ccaatgggaa tgaagacacc acagctgatt tcttcctgac cactatgccc actgactccc
tcagtgtttc cactctgccc ctcccagagg ttcagtgttt tgtgttcaat gtcgagtaca
tgaattgcac ttggaacagc agctctgagc cccagcctac caacctcact ctgcattatt
ggtacaagaa ctcggataat gataaagtcc agaagtgcag ccactatcta ttctctgaag
aaatcacttc tggctgtcag ttgcaaaaaa aggagatcca cctctaccaa acatttgttg
ttcagctcca ggacccacgg gaacccagga gacaggccac acagatgcta aaactgcaga
atctggtgat cccctgggct ccagagaacc taacacttca caaactgagt gaatcccagc
tagaactgaa ctggaacaac agattcttga accactgttt ggagcacttg gtgcagtacc
ggactgactg ggaccacagc tggactgaac aatcagtgga ttatagacat aagttctcct
tgcctagtgt ggatgggcag aaacgctaca cgtttcgtgt tcggagccgc tttaacccac
tctgtggaag tgctcagcat tggagtgaat ggagccaccc aatccactgg gggagcaata
cttcaaaaga gaatcctttc ctgtttgcat tggaagccgt ggttatctct gttggctcca
tgggattgat tatcagcctt ctctgtgtgt atttctggct ggaacggacg atgccccgaa
ttcccaccct gaagaaccta gaggatcttg ttactgaata ccacgggaac ttttcggcct
ggagtggtgt gtctaaggga ctggctgaga gtctgcagcc agactacagt gaacgactct
gcctcgtcag tgagattccc ccaaaaggag gggcccttgg ggaggggcct ggggcctccc
catgcaacca gcatagcccc tactgggccc ccccatgtta caccctaaag cctgaaacct
gaaccccaat cctctgacag aagaacccca gggtcctgta gccctaagtg gtactaactt
tccttcattc aacccacctg cgtctcatac tcacctcacc ccactgtggc tgatttggaa
ttttgtgccc ccatgtaagc accccttcat ttggcattcc ccacttgaga attacccttt
tgccccgaac atgtttttct tctccctcag tctggccctt ccttttcgca ggattcttcc
tccctccctc tttccctccc ttcctctttc catctaccct ccgattgttc ctgaaccgat
gagaaataaa gtttctgttg ataatcatca aaaaaaaaaa aaaaaaaaaa aaaaaaaaaa
IL-15: The human IL-15 sequence is represented by GenBank accession number
NM_172175.2 (SEQ ID NO: 5) listed below:
gttgggactc cgggtggcag gcgcccgggg gaatcccagc tgactcgctc actgccttcg
aagtccggcg ccccccggga gggaactggg tggccgcacc ctcccggctg cggtggctgt
cgccccccac cctgcagcca ggactcgatg gagaatccat tccaatatat ggccatgtgg
ctctttggag caatgttcca tcatgttcca tgctgctgac gtcacatgga gcacagaaat
caatgttagc agatagccag cccatacaag atcgttttca actagtggcc ccactgtgtc
cggaattgat gggttcttgg tctcactgac ttcaagaatg aagccgcgga ccctcgcggt
gagtgttaca gctcttaagg tggcgcatct ggagtttgtt ccttctgatg ttcggatgtg
ttcggagttt cttccttctg gtgggttcgt ggtctcgctg gctcaggagt gaagctacag
accttcgcgg aggcattgtg gatggatggc tgctggaaac cccttgccat agccagctct
tcttcaatac ttaaggattt accgtggctt tgagtaatga gaatttcgaa accacatttg
agaagtattt ccatccagtg ctacttgtgt ttacttctaa acagtcattt tctaactgaa
gctggcattc atgtcttcat tttgggatgc agctaatata cccagttggc ccaaagcacc
taacctatag ttatataatc tgactctcag ttcagtttta ctctactaat gccttcatgg
tattgggaac catagatttg tgcagctgtt tcagtgcagg gcttcctaaa acagaagcca
actgggtgaa tgtaataagt gatttgaaaa aaattgaaga tcttattcaa tctatgcata
ttgatgctac tttatatacg gaaagtgatg ttcaccccag ttgcaaagta acagcaatga
agtgctttct cttggagtta caagttattt cacttgagtc cggagatgca agtattcatg
atacagtaga aaatctgatc atcctagcaa acaacagttt gtcttctaat gggaatgtaa
cagaatctgg atgcaaagaa tgtgaggaac tggaggaaaa aaatattaaa gaatttttgc
agagttttgt acatattgtc caaatgttca tcaacacttc ttgattgcaa ttgattcttt
ttaaagtgtt tctgttatta acaaacatca ctctgctgct tagacataac aaaacactcg
gcatttcaaa tgtgctgtca aaacaagttt ttctgtcaag aagatgatca gaccttggat
cagatgaact cttagaaatg aaggcagaaa aatgtcattg agtaatatag tgactatgaa
cttctctcag acttacttta ctcatttttt taatttatta ttgaaattgt acatatttgt
ggaataatgt aaaatgttga ataaaaatat gtacaagtgt tgttttttaa gttgcactga
tattttacct cttattgcaa aatagcattt gtttaagggt gatagtcaaa ttatgtattg
gtggggctgg gtaccaatgc tgcaggtcaa cagctatgct ggtaggctcc tgccagtgtg
gaaccactga ctactggctc tcattgactt ccttactaag catagcaaac agaggaagaa
tttgttatca gtaagaaaaa gaagaactat atgtgaatcc tcttctttat actgtaattt
agttattgat gtataaagca actgttatga aataaagaaa ttgcaataac tggcatataa
tgtccatcag taaatcttgg tggtggtggc aataataaac ttctactgat aggtagaatg
gtgtgcaagc ttgtccaatc acggattgca ggccacatgc ggcccaggac aactttgaat
gtggcccaac acaaattcat aaactttcat acatctcgtt tttagctcat cagctatcat
tagcggtagt gtatttaaag tgtggcccaa gacaattctt cttattccaa tgtggcccag
ggaaatcaaa agattggatg cccctggtat agaaaactaa tagtgacagt gttcatattt
catgctttcc caaatacagg tattttattt tcacattctt tttgccatgt ttatataata
ataaagaaaa accctgttga tttgttggag ccattgttat ctgacagaaa ataattgttt
atattttttg cactacactg tctaaaatta gcaagctctc ttctaatgga actgtaagaa
agatgaaata tttttgtttt attataaatt tatttcacct taaaaaaaaa aaa
IL-15Ra: The human IL-15Ra sequence is represented by GenBank accession number
NM_001243539 (SEQ ID NO: 6) listed below:
agctgcagca ggaattcggc gaagtggcgg agctggggcc ccagcgggcg ccgggggccg
cgggagccag caggtggcgg gggctgcgct ccgcccgggc cagagcgcac caggcaggtg
cccgcgcctc cgcaccgcgg cgacacctcc gcgggcactc acccaggccg gccgctcaca
accgagcgca gggccgcgga gggagaccag gaaagccgaa ggcggagcag ctggaggcga
ccagcgccgg gcgaggtcaa gtggatccga gccgcagaga gggctggaga gagtctgctc
tccgatgact ttgcccactc tcttcgcagt ggggacaccg gaccgagtgc acactggagg
tcccagagca cgacgagcgc ggaggaccgg gaggctcccg ggcttgcgtg ggcatcacgt
gccctccccc catgtccgtg gaacacgcag acatctgggt caagagctac agcttgtact
ccagggagcg gtacatttgt aactctggtt tcaagcgtaa agccggcacg tccagcctga
cggagtgcgt gttgaacaag gccacgaatg tcgcccactg gacaaccccc agtctcaaat
gcattagaga ccctgccctg gttcaccaaa ggccagcgcc accctccaca gtaacgacgg
caggggtgac cccacagcca gagagcctct ccccttctgg aaaagagccc gcagcttcat
ctcccagctc aaacaacaca gcggccacaa cagcagctat tgtcccgggc tcccagctga
tgccttcaaa atcaccttcc acaggaacca cagagataag cagtcatgag tcctcccacg
gcaccccctc tcagacaaca gccaagaact gggaactcac agcatccgcc tcccaccagc
cgccaggtgt gtatccacag ggccacagcg acaccactgt ggctatctcc acgtccactg
tcctgctgtg tgggctgagc gctgtgtctc tcctggcatg ctacctcaag tcaaggcaaa
ctcccccgct ggccagcgtt gaaatggaag ccatggaggc tctgccggtg acttggggga
ccagcagcag agatgaagac ttggaaaact gctctcacca cctatgaaac tcggggaaac
cagcccagct aagtccggag tgaaggagcc tctctgcttt agctaaagac gactgagaag
aggtgcaagg aagcgggctc caggagcaag ctcaccaggc ctctcagaag tcccagcagg
atctcacgga ctgccgggtc ggcgcctcct gcgcgaggga gcaggttctc cgcattccca
tgggcaccac ctgcctgcct gtcgtgcctt ggacccaggg cccagcttcc caggagagac
tgagcaggat ttttatttca ttacagtgtg agctgcctgg aatacatgtg gtaatgaaat
aaaaaccctg ccccgaatct tccgtccctc atcctaactt tcagttcaca gagaaaagtg
acatacccaa agctctctgt caattacaag gcttctcctg gcgtgggaga cgtctacagg
gaagacacca gcgtttgggc ttctaaccac cctgtctcca gctgctctgc acacatggac
agggacctgg gaaaggtggg agagatgctg agcccagcga atcctctcca ttgaaggatt
caggaagaag aaaactcaac tcagtgccat tttacgaata tatgcgttta tatttatact
tccttgtcta ttatatctat acattatata ttatttgtat tttgacattg aaacaaaata
aaacatctat tttcaatatt tttaaaatgc aaaaaaaaaa a
IL-12α: The human IL-12 α sequence is represented by GenBank accession number
NM_000882.3 (SEQ ID NO:7) listed below:
tttcgctttc attttgggcc gagctggagg cggcggggcc gtcccggaac ggctgcggcc
gggcaccccg ggagttaatc cgaaagcgcc gcaagccccg cgggccggcc gcaccgcacg
tgtcaccgag aagctgatgt agagagagac acagaaggag acagaaagca agagaccaga
gtcccgggaa agtcctgccg cgcctcggga caattataaa aatgtggccc cctgggtcag
cctcccagcc accgccctca cctgccgcgg ccacaggtct gcatccagcg gctcgccctg
tgtccctgca gtgccggctc agcatgtgtc cagcgcgcag cctcctcctt gtggctaccc
tggtcctcct ggaccacctc agtttggcca gaaacctccc cgtggccact ccagacccag
gaatgttccc atgccttcac cactcccaaa acctgctgag ggccgtcagc aacatgctcc
agaaggccag acaaactcta gaattttacc cttgcacttc tgaagagatt gatcatgaag
atatcacaaa agataaaacc agcacagtgg aggcctgttt accattggaa ttaaccaaga
atgagagttg cctaaattcc agagagacct ctttcataac taatgggagt tgcctggcct
ccagaaagac ctcttttatg atggccctgt gccttagtag tatttatgaa gacttgaaga
tgtaccaggt ggagttcaag accatgaatg caaagcttct gatggatcct aagaggcaga
tctttctaga tcaaaacatg ctggcagtta ttgatgagct gatgcaggcc ctgaatttca
acagtgagac tgtgccacaa aaatcctccc ttgaagaacc ggatttttat aaaactaaaa
tcaagctctg catacttctt catgctttca gaattcgggc agtgactatt gatagagtga
tgagctatct gaatgcttcc taaaaagcga ggtccctcca aaccgttgtc atttttataa
aactttgaaa tgaggaaact ttgataggat gtggattaag aactagggag ggggaaagaa
ggatgggact attacatcca catgatacct ctgatcaagt atttttgaca tttactgtgg
ataaattgtt tttaagtttt catgaatgaa ttgctaagaa gggaaaatat ccatcctgaa
ggtgtttttc attcacttta atagaagggc aaatatttat aagctatttc tgtaccaaag
tgtttgtgga aacaaacatg taagcataac ttattttaaa atatttattt atataacttg
gtaatcatga aagcatctga gctaacttat atttatttat gttatattta ttaaattatt
tatcaagtgt atttgaaaaa tatttttaag tgttctaaaa ataaaagtat tgaattaaag
tgaaaaaaaa
IL-12β: The human IL-12 β sequence is represented by GenBank accession number
NM_002187.2.3 (SEQ ID NO: 8) listed below:
ctgtttcagg gccattggac tctccgtcct gcccagagca agatgtgtca ccagcagttg
gtcatctctt ggttttccct ggtttttctg gcatctcccc tcgtggccat atgggaactg
aagaaagatg tttatgtcgt agaattggat tggtatccgg atgcccctgg agaaatggtg
gtcctcacct gtgacacccc tgaagaagat ggtatcacct ggaccttgga ccagagcagt
gaggtcttag gctctggcaa aaccctgacc atccaagtca aagagtttgg agatgctggc
cagtacacct gtcacaaagg aggcgaggtt ctaagccatt cgctcctgct gcttcacaaa
aaggaagatg gaatttggtc cactgatatt ttaaaggacc agaaagaacc caaaaataag
acctttctaa gatgcgaggc caagaattat tctggacgtt tcacctgctg gtggctgacg
acaatcagta ctgatttgac attcagtgtc aaaagcagca gaggctcttc tgacccccaa
ggggtgacgt gcggagctgc tacactctct gcagagagag tcagagggga caacaaggag
tatgagtact cagtggagtg ccaggaggac agtgcctgcc cagctgctga ggagagtctg
cccattgagg tcatggtgga tgccgttcac aagctcaagt atgaaaacta caccagcagc
ttcttcatca gggacatcat caaacctgac ccacccaaga acttgcagct gaagccatta
aagaattctc ggcaggtgga ggtcagctgg gagtaccctg acacctggag tactccacat
tcctacttct ccctgacatt ctgcgttcag gtccagggca agagcaagag agaaaagaaa
gatagagtct tcacggacaa gacctcagcc acggtcatct gccgcaaaaa tgccagcatt
agcgtgcggg cccaggaccg ctactatagc tcatcttgga gcgaatgggc atctgtgccc
tgcagttagg ttctgatcca ggatgaaaat ttggaggaaa agtggaagat attaagcaaa
atgtttaaag acacaacgga atagacccaa aaagataatt tctatctgat ttgctttaaa
acgttttttt aggatcacaa tgatatcttt gctgtatttg tatagttaga tgctaaatgc
tcattgaaac aatcagctaa tttatgtata gattttccag ctctcaagtt gccatgggcc
ttcatgctat ttaaatattt aagtaattta tgtatttatt agtatattac tgttatttaa
cgtttgtctg ccaggatgta tggaatgttt catactctta tgacctgatc catcaggatc
agtccctatt atgcaaaatg tgaatttaat tttatttgta ctgacaactt ttcaagcaag
gctgcaagta catcagtttt atgacaatca ggaagaatgc agtgttctga taccagtgcc
atcatacact tgtgatggat gggaacgcaa gagatactta catggaaacc tgacaatgca
aacctgttga gaagatccag gagaacaaga tgctagttcc catgtctgtg aagacttcct
ggagatggtg ttgataaagc aatttagggc cacttacact tctaagcaag tttaatcttt
ggatgcctga attttaaaag ggctagaaaa aaatgattga ccagcctggg aaacataaca
agaccccgtc tctacaaaaa aaatttaaaa ttagccaggc gtggtggctc atgcttgtgg
tcccagctgt tcaggaggat gaggcaggag gatctcttga gcccaggagg tcaaggctat
ggtgagccgt gattgtgcca ctgcatacca gcctaggtga cagaatgaga ccctgtctca
aaaaaaaaaa tgattgaaat taaaattcag ctttagcttc catggcagtc ctcaccccca
cctctctaaa agacacagga ggatgacaca gaaacaccgt aagtgtctgg aaggcaaaaa
gatcttaaga ttcaagagag aggacaagta gttatggcta aggacatgaa attgtcagaa
tggcaggtgg cttcttaaca gccctgtgag aagcagacag atgcaaagaa aatctggaat
ccctttctca ttagcatgaa tgaacctgat acacaattat gaccagaaaa tatggctcca
tgaaggtgct acttttaagt aatgtatgtg cgctctgtaa agtgattaca tttgtttcct
gtttgtttat ttatttattt atttttgcat tctgaggctg aactaataaa aactcttctt
tgtaatc
IL-12Rβ1: The human IL-12Rβ1 sequence is represented by GenBank accession number
NM_005535 (SEQ ID NO: 9) listed below:
ctctttcact ttgacttgcc ttagggatgg gctgtgacac tttacttttt ttcttttttc
ttttttttca gtcttttctc cttgctcagc ttcaatgtgt tccggagtgg ggacggggtg
gctgaacctc gcaggtggca gagaggctcc cctggggctg tggggctcta cgtggatccg
atggagccgc tggtgacctg ggtggtcccc ctcctcttcc tcttcctgct gtccaggcag
ggcgctgcct gcagaaccag tgagtgctgt tttcaggacc cgccatatcc ggatgcagac
tcaggctcgg cctcgggccc tagggacctg agatgctatc ggatatccag tgatcgttac
gagtgctcct ggcagtatga gggtcccaca gctggggtca gccacttcct gcggtgttgc
cttagctccg ggcgctgctg ctacttcgcc gccggctcag ccaccaggct gcagttctcc
gaccaggctg gggtgtctgt gctgtacact gtcacactct gggtggaatc ctgggccagg
aaccagacag agaagtctcc tgaggtgacc ctgcagctct acaactcagt taaatatgag
cctcctctgg gagacatcaa ggtgtccaag ttggccgggc agctgcgtat ggagtgggag
accccggata accaggttgg tgctgaggtg cagttccggc accggacacc cagcagccca
tggaagttgg gcgactgcgg acctcaggat gatgatactg agtcctgcct ctgccccctg
gagatgaatg tggcccagga attccagctc cgacgacggc agctggggag ccaaggaagt
tcctggagca agtggagcag ccccgtgtgc gttccccctg aaaacccccc acagcctcag
gtgagattct cggtggagca gctgggccag gatgggagga ggcggctgac cctgaaagag
cagccaaccc agctggagct tccagaaggc tgtcaagggc tggcgcctgg cacggaggtc
acttaccgac tacagctcca catgctgtcc tgcccgtgta aggccaaggc caccaggacc
ctgcacctgg ggaagatgcc ctatctctcg ggtgctgcct acaacgtggc tgtcatctcc
tcgaaccaat ttggtcctgg cctgaaccag acgtggcaca ttcctgccga cacccacaca
gaaccagtgg ctctgaatat cagcgtcgga accaacggga ccaccatgta ttggccagcc
cgggctcaga gcatgacgta ttgcattgaa tggcagcctg tgggccagga cgggggcctt
gccacctgca gcctgactgc gccgcaagac ccggatccgg ctggaatggc aacctacagc
tggagtcgag agtctggggc aatggggcag gaaaagtgtt actacattac catctttgcc
tctgcgcacc ccgagaagct caccttgtgg tctacggtcc tgtccaccta ccactttggg
ggcaatgcct cagcagctgg gacaccgcac cacgtctcgg tgaagaatca tagcttggac
tctgtgtctg tggactgggc accatccctg ctgagcacct gtcccggcgt cctaaaggag
tatgttgtcc gctgccgaga tgaagacagc aaacaggtgt cagagcatcc cgtgcagccc
acagagaccc aagttaccct cagtggcctg cgggctggtg tagcctacac ggtgcaggtg
cgagcagaca cagcgtggct gaggggtgtc tggagccagc cccagcgctt cagcatcgaa
gtgcaggttt ctgattggct catcttcttc gcctccctgg ggagcttcct gagcatcctt
ctcgtgggcg tccttggcta ccttggcctg aacagggccg cacggcacct gtgcccgccg
ctgcccacac cctgtgccag ctccgccatt gagttccctg gagggaagga gacttggcag
tggatcaacc cagtggactt ccaggaagag gcatccctgc aggaggccct ggtggtagag
atgtcctggg acaaaggcga gaggactgag cctctcgaga agacagagct acctgagggt
gcccctgagc tggccctgga tacagagttg tccttggagg atggagacag gtgcaaggcc
aagatgtgat cgttgaggct cagagagggt gagtgactcg cccgaggcta cgtagcacac
acaggagtca catttggacc caaataaccc agagctcctc caggctccag tgcacctgcc
tcctctctgc cccgtgcctg ttgccaccca tcctgcgggg gaaccctaga tgctgccatg
aaatggaagc tgctgcaccc tgctgggcct ggcatccgtg gggcaggagc agaccctgcc
atttacctgt tctggcgtag aatggactgg gaatgggggc aaggggggct cagatggatc
cctggaccct gggctgggca tccaccccca ggagcactgg atggggagtc tggactcaag
ggctccctgc agcattgcgg ggtcttgtag cttggaggat ccaggcatat agggaagggg
gctgtaaact ttgtgggaaa aatgacggtc ctcccatccc accccccacc ccaccctcac
ccccctataa aatgggggtg gtgataatga ccttacacag ctgttcaaaa tcatcgtaaa
tgagcctcct cttgggtatt tttttcctgt ttgaagcttg aatgtcctgc tcaaaatctc
aaaacacgag ccttggaatt caaaaaaaaa aaaaaaaaaa
IL-12Rβ2: The human IL-12Rβ2 sequence is represented by GenBank accession number
NM_001559.2 (SEQ ID NO: 10) listed below:
tgcagagcac agagaaagga catctgcgag gaaagttccc tgatggctgt caacaaagtg
ccacgtctct atggctgtga acgctgagca cacgatttta tcgcgcctat catatcttgg
tgcataaacg cacctcacct cggtcaaccc ttgctccgtc ttatgagaca ggctttatta
tccgcatttt atatgagggg aaactgacgg tggagagaga attatcttgc tcaaggcgac
acagcagagc ccacaggtgg cagaatccca cccgagcccg cttcgacccg cggggtggaa
accacgggcg cccgcccggc tgcgcttcca gagctgaact gagaagcgag tcctctccgc
cctgcggcca ccgcccagcc ccgacccccg ccccggcccg atcctcactc gccgccagct
ccccgcgccc accccggagt tggtggcgca gaggcgggag gcggaggcgg gagggcgggc
gctggcaccg ggaacgcccg agcgccggca gagagcgcgg agagcgcgac acgtgcggcc
cagagcaccg gggccacccg gtccccgcag gcccgggacc gcgcccgctg gcaggcgaca
cgtggaagaa tacggagttc tataccagag ttgattgttg atggcacata cttttagagg
atgctcattg gcatttatgt ttataatcac gtggctgttg attaaagcaa aaatagatgc
gtgcaagaga ggcgatgtga ctgtgaagcc ttcccatgta attttacttg gatccactgt
caatattaca tgctctttga agcccagaca aggctgcttt cactattcca gacgtaacaa
gttaatcctg tacaagtttg acagaagaat caattttcac catggccact ccctcaattc
tcaagtcaca ggtcttcccc ttggtacaac cttgtttgtc tgcaaactgg cctgtatcaa
tagtgatgaa attcaaatat gtggagcaga gatcttcgtt ggtgttgctc cagaacagcc
tcaaaattta tcctgcatac agaagggaga acaggggact gtggcctgca cctgggaaag
aggacgagac acccacttat acactgagta tactctacag ctaagtggac caaaaaattt
aacctggcag aagcaatgta aagacattta ttgtgactat ttggactttg gaatcaacct
cacccctgaa tcacctgaat ccaatttcac agccaaggtt actgctgtca atagtcttgg
aagctcctct tcacttccat ccacattcac attcttggac atagtgaggc ctcttcctcc
gtgggacatt agaatcaaat ttcaaaaggc ttctgtgagc agatgtaccc tttattggag
agatgaggga ctggtactgc ttaatcgact cagatatcgg cccagtaaca gcaggctctg
gaatatggtt aatgttacaa aggccaaagg aagacatgat ttgctggatc tgaaaccatt
tacagaatat gaatttcaga tttcctctaa gctacatctt tataagggaa gttggagtga
ttggagtgaa tcattgagag cacaaacacc agaagaagag cctactggga tgttagatgt
ctggtacatg aaacggcaca ttgactacag tagacaacag atttctcttt tctggaagaa
tctgagtgtc tcagaggcaa gaggaaaaat tctccactat caggtgacct tgcaggagct
gacaggaggg aaagccatga cacagaacat cacaggacac acctcctgga ccacagtcat
tcctagaacc ggaaattggg ctgtggctgt gtctgcagca aattcaaaag gcagttctct
gcccactcgt attaacataa tgaacctgtg tgaggcaggg ttgctggctc ctcgccaggt
ctctgcaaac tcagagggca tggacaacat tctggtgact tggcagcctc ccaggaaaga
tccctctgct gttcaggagt acgtggtgga atggagagag ctccatccag ggggtgacac
acaggtccct ctaaactggc tacggagtcg accctacaat gtgtctgctc tgatttcaga
gaacataaaa tcctacatct gttatgaaat ccgtgtgtat gcactctcag gggatcaagg
aggatgcagc tccatcctgg gtaactctaa gcacaaagca ccactgagtg gcccccacat
taatgccatc acagaggaaa aggggagcat tttaatttca tggaacagca ttccagtcca
ggagcaaatg ggctgcctcc tccattatag gatatactgg aaggaacggg actccaactc
ccagcctcag ctctgtgaaa ttccctacag agtctcccaa aattcacatc caataaacag
cctgcagccc cgagtgacat atgtcctgtg gatgacagct ctgacagctg ctggtgaaag
ttcccacgga aatgagaggg aattttgtct gcaaggtaaa gccaattgga tggcgtttgt
ggcaccaagc atttgcattg ctatcatcat ggtgggcatt ttctcaacgc attacttcca
gcaaaaggtg tttgttctcc tagcagccct cagacctcag tggtgtagca gagaaattcc
agatccagca aatagcactt gcgctaagaa atatcccatt gcagaggaga agacacagct
gcccttggac aggctcctga tagactggcc cacgcctgaa gatcctgaac cgctggtcat
cagtgaagtc cttcatcaag tgaccccagt tttcagacat cccccctgct ccaactggcc
acaaagggaa aaaggaatcc aaggtcatca ggcctctgag aaagacatga tgcacagtgc
ctcaagccca ccacctccaa gagctctcca agctgagagc agacaactgg tggatctgta
caaggtgctg gagagcaggg gctccgaccc aaagcccgaa aacccagcct gtccctggac
ggtgctccca gcaggtgacc ttcccaccca tgatggctac ttaccctcca acatagatga
cctcccctca catgaggcac ctctcgctga ctctctggaa gaactggagc ctcagcacat
ctccctttct gttttcccct caagttctct tcacccactc accttctcct gtggtgataa
gctgactctg gatcagttaa agatgaggtg tgactccctc atgctctgag tggtgaggct
tcaagcctta aagtcagtgt gccctcaacc agcacagcct gccccaattc ccccagcccc
tgctccagca gctgtcatct ctgggtgcca ccatcggtct ggctgcagct agaggacagg
caagccagct ctgggggagt cttaggaact gggagttggt cttcactcag atgcctcatc
ttgcctttcc cagggcctta aaattacatc cttcactgtg tggacctaga gactccaact
tgaattccta gtaactttct tggtatgctg gccagaaagg gaaatgagga ggagagtaga
aaccacagct cttagtagta atggcataca gtctagagga ccattcatgc aatgactatt
tctaaagcac ctgctacaca gcaggctgta cacagcagat cagtactgtt caacagaact
tcctgagatg atggaaatgt tctacctctg cactcactgt ccagtacatt agacactagg
cacattggct gttaatcact tggaatgtgt ttagcttgac tgaggaatta aattttgatt
gtaaatttaa atcgccacac atggctagtg gctactgtat tggagtgcac agctctagat
ggctcctaga ttattgagag ccttcaaaac aaatcaacct agttctatag atgaagacat
aaaagacact ggtaaacacc aaggtaaaag ggcccccaag gtggtcatga ctggtctcat
ttgcagaagt ctaagaatgt acctttttct ggccgggcgt ggtagctcat gcctgtaatc
ccagcacttt gggaggctga
IL-17a: The human IL-17a sequence is represented by GenBank accession number
NM_002190.2 (SEQ ID NO: 11) listed below:
gcaggcacaa actcatccat ccccagttga ttggaagaaa caacgatgac tcctgggaag
acctcattgg tgtcactgct actgctgctg agcctggagg ccatagtgaa ggcaggaatc
acaatcccac gaaatccagg atgcccaaat tctgaggaca agaacttccc ccggactgtg
atggtcaacc tgaacatcca taaccggaat accaatacca atcccaaaag gtcctcagat
tactacaacc gatccacctc accttggaat ctccaccgca atgaggaccc tgagagatat
ccctctgtga tctgggaggc aaagtgccgc cacttgggct gcatcaacgc tgatgggaac
gtggactacc acatgaactc tgtccccatc cagcaagaga tcctggtcct gcgcagggag
cctccacact gccccaactc cttccggctg gagaagatac tggtgtccgt gggctgcacc
tgtgtcaccc cgattgtcca ccatgtggcc taagagctct ggggagccca cactccccaa
agcagttaga ctatggagag ccgacccagc ccctcaggaa ccctcatcct tcaaagacag
cctcatttcg gactaaactc attagagttc ttaaggcagt ttgtccaatt aaagcttcag
aggtaacact tggccaagat atgagatctg aattaccttt ccctctttcc aagaaggaag
gtttgactga gtaccaattt gcttcttgtt tactttttta agggctttaa gttatttatg
tatttaatat gccctgagat aactttgggg tataagattc cattttaatg aattacctac
tttattttgt ttgtcttttt aaagaagata agattctggg cttgggaatt ttattattta
aaaggtaaaa cctgtattta tttgagctat ttaaggatct atttatgttt aagtatttag
aaaaaggtga aaaagcacta ttatcagttc tgcctaggta aatgtaagat agaattaaat
ggcagtgcaa aatttctgag tctttacaac atacggatat agtatttcct cctctttgtt
tttaaaagtt ataacatggc tgaaaagaaa gattaaacct actttcatat gtattaattt
aaattttgca atttgttgag gttttacaag agatacagca agtctaactc tctgttccat
taaaccctta taataaaatc cttctgtaat aataaagttt caaaagaaaa tgtttatttg
ttctcattaa atgtatttta gcaaactcag ctcttcccta ttgggaagag ttatgcaaat
tctcctataa gcaaaacaaa gcatgtcttt gagtaacaat gacctggaaa tacccaaaat
tccaagttct cgatttcaca tgccttcaag actgaacacc gactaaggtt ttcatactat
tagccaatgc tgtagacaga agcattttga taggaataga gcaaataaga taatggccct
gaggaatggc atgtcattat taaagatcat atggggaaaa tgaaaccctc cccaaaatac
aagaagttct gggaggagac attgtcttca gactacaatg tccagtttct cccctagact
caggcttcct ttggagatta aggcccctca gagatcaaca gaccaacatt tttctcttcc
tcaagcaaca ctcctagggc ctggcttctg tctgatcaag gcaccacaca acccagaaag
gagctgatgg ggcagaacga actttaagta tgagaaaagt tcagcccaag taaaataaaa
actcaatcac attcaattcc agagtagttt caagtttcac atcgtaacca ttttcgccc
IFN-gamma: The human IFN-gamma sequence is represented by GenBank accession
number NM_000619.2 (SEQ ID NO: 12) listed below:
cacattgttc tgatcatctg aagatcagct attagaagag aaagatcagt taagtccttt
ggacctgatc agcttgatac aagaactact gatttcaact tctttggctt aattctctcg
gaaacgatga aatatacaag ttatatcttg gcttttcagc tctgcatcgt tttgggttct
cttggctgtt actgccagga cccatatgta aaagaagcag aaaaccttaa gaaatatttt
aatgcaggtc attcagatgt agcggataat ggaactcttt tcttaggcat tttgaagaat
tggaaagagg agagtgacag aaaaataatg cagagccaaa ttgtctcctt ttacttcaaa
ctttttaaaa actttaaaga tgaccagagc atccaaaaga gtgtggagac catcaaggaa
gacatgaatg tcaagttttt caatagcaac aaaaagaaac gagatgactt cgaaaagctg
actaattatt cggtaactga cttgaatgtc caacgcaaag caatacatga actcatccaa
gtgatggctg aactgtcgcc agcagctaaa acagggaagc gaaaaaggag tcagatgctg
tttcgaggtc gaagagcatc ccagtaatgg ttgtcctgcc tgcaatattt gaattttaaa
tctaaatcta tttattaata tttaacatta tttatatggg gaatatattt ttagactcat
caatcaaata agtatttata atagcaactt ttgtgtaatg aaaatgaata tctattaata
tatgtattat ttataattcc tatatcctgt gactgtctca cttaatcctt tgttttctga
ctaattaggc aaggctatgt gattacaagg ctttatctca ggggccaact aggcagccaa
cctaagcaag atcccatggg ttgtgtgttt atttcacttg atgatacaat gaacacttat
aagtgaagtg atactatcca gttactgccg gtttgaaaat atgcctgcaa tctgagccag
tgctttaatg gcatgtcaga cagaacttga atgtgtcagg tgaccctgat gaaaacatag
catctcagga gatttcatgc ctggtgcttc caaatattgt tgacaactgt gactgtaccc
aaatggaaag taactcattt gttaaaatta tcaatatcta atatatatga ataaagtgta
agttcacaac aaaaaaaaaa aaaaaaaaaa aaaaaaaaaa
CD28: The human CD28 sequence is represented by GenBank accession number
NM_006139.3 (SEQ ID NO: 13) listed below:
taaagtcatc aaaacaacgt tatatcctgt gtgaaatgct gcagtcagga tgccttgtgg
tttgagtgcc ttgatcatgt gccctaaggg gatggtggcg gtggtggtgg ccgtggatga
cggagactct caggccttgg caggtgcgtc tttcagttcc cctcacactt cgggttcctc
ggggaggagg ggctggaacc ctagcccatc gtcaggacaa agatgctcag gctgctcttg
gctctcaact tattcccttc aattcaagta acaggaaaca agattttggt gaagcagtcg
cccatgcttg tagcgtacga caatgcggtc aaccttagct gcaagtattc ctacaatctc
ttctcaaggg agttccgggc atcccttcac aaaggactgg atagtgctgt ggaagtctgt
gttgtatatg ggaattactc ccagcagctt caggtttact caaaaacggg gttcaactgt
gatgggaaat tgggcaatga atcagtgaca ttctacctcc agaatttgta tgttaaccaa
acagatattt acttctgcaa aattgaagtt atgtatcctc ctccttacct agacaatgag
aagagcaatg gaaccattat ccatgtgaaa gggaaacacc tttgtccaag tcccctattt
cccggacctt ctaagccctt ttgggtgctg gtggtggttg gtggagtcct ggcttgctat
agcttgctag taacagtggc ctttattatt ttctgggtga ggagtaagag gagcaggctc
ctgcacagtg actacatgaa catgactccc cgccgccccg ggcccacccg caagcattac
cagccctatg ccccaccacg cgacttcgca gcctatcgct cctgacacgg acgcctatcc
agaagccagc cggctggcag cccccatctg ctcaatatca ctgctctgga taggaaatga
ccgccatctc cagccggcca cctcaggccc ctgttgggcc accaatgcca atttttctcg
agtgactaga ccaaatatca agatcatttt gagactctga aatgaagtaa aagagatttc
ctgtgacagg ccaagtctta cagtgccatg gcccacattc caacttacca tgtacttagt
gacttgactg agaagttagg gtagaaaaca aaaagggagt ggattctggg agcctcttcc
ctttctcact cacctgcaca tctcagtcaa gcaaagtgtg gtatccacag acattttagt
tgcagaagaa aggctaggaa atcattcctt ttggttaaat gggtgtttaa tcttttggtt
agtgggttaa acggggtaag ttagagtagg gggagggata ggaagacata tttaaaaacc
attaaaacac tgtctcccac tcatgaaatg agccacgtag ttcctattta atgctgtttt
cctttagttt agaaatacat agacattgtc ttttatgaat tctgatcata tttagtcatt
ttgaccaaat gagggatttg gtcaaatgag ggattccctc aaagcaatat caggtaaacc
aagttgcttt cctcactccc tgtcatgaga cttcagtgtt aatgttcaca atatactttc
gaaagaataa aatagttctc ctacatgaag aaagaatatg tcaggaaata aggtcacttt
atgtcaaaat tatttgagta ctatgggacc tggcgcagtg gctcatgctt gtaatcccag
cactttggga ggccgaggtg ggcagatcac ttgagatcag gaccagcctg gtcaagatgg
tgaaactccg tctgtactaa aaatacaaaa tttagcttgg cctggtggca ggcacctgta
atcccagctg cccaagaggc tgaggcatga gaatcgcttg aacctggcag gcggaggttg
cagtgagccg agatagtgcc acagctctcc agcctgggcg acagagtgag actccatctc
aaacaacaac aacaacaaca acaacaacaa caaaccacaa aattatttga gtactgtgaa
ggattatttg tctaacagtt cattccaatc agaccaggta ggagctttcc tgtttcatat
gtttcagggt tgcacagttg gtctctttaa tgtcggtgtg gagatccaaa gtgggttgtg
gaaagagcgt ccataggaga agtgagaata ctgtgaaaaa gggatgttag cattcattag
agtatgagga tgagtcccaa gaaggttctt tggaaggagg acgaatagaa tggagtaatg
aaattcttgc catgtgctga ggagatagcc agcattaggt gacaatcttc cagaagtggt
caggcagaag gtgccctggt gagagctcct ttacagggac tttatgtggt ttagggctca
gagctccaaa actctgggct cagctgctcc tgtaccttgg aggtccattc acatgggaaa
gtattttgga atgtgtcttt tgaagagagc atcagagttc ttaagggact gggtaaggcc
tgaccctgaa atgaccatgg atatttttct acctacagtt tgagtcaact agaatatgcc
tggggacctt gaagaatggc ccttcagtgg ccctcaccat ttgttcatgc ttcagttaat
tcaggtgttg aaggagctta ggttttagag gcacgtagac ttggttcaag tctcgttagt
agttgaatag cctcaggcaa gtcactgccc acctaagatg atggttcttc aactataaaa
tggagataat ggttacaaat gtctcttcct atagtataat ctccataagg gcatggccca
agtctgtctt tgactctgcc tatccctgac atttagtagc atgcccgaca tacaatgtta
gctattggta ttattgccat atagataaat tatgtataaa aattaaactg ggcaatagcc
taagaagggg ggaatattgt aacacaaatt taaacccact acgcagggat gaggtgctat
aatatgagga ccttttaact tccatcattt tcctgtttct tgaaatagtt tatcttgtaa
tgaaatataa ggcacctccc acttttatgt atagaaagag gtcttttaat ttttttttaa
tgtgagaagg aagggaggag taggaatctt gagattccag atcgaaaata ctgtactttg
gttgattttt aagtgggctt ccattccatg gatttaatca gtcccaagaa gatcaaactc
agcagtactt gggtgctgaa gaactgttgg atttaccctg gcacgtgtgc cacttgccag
cttcttgggc acacagagtt cttcaatcca agttatcaga ttgtatttga aaatgacaga
gctggagagt tttttgaaat ggcagtggca aataaataaa tacttttttt taaatggaaa
gacttgatct atggtaataa atgattttgt tttctgactg gaaaaatagg cctactaaag
atgaatcaca cttgagatgt ttcttactca ctctgcacag aaacaaagaa gaaatgttat
acagggaagt ccgttttcac tattagtatg aaccaagaaa tggttcaaaa acagtggtag
gagcaatgct ttcatagttt cagatatggt agttatgaag aaaacaatgt catttgctgc
tattattgta agagtcttat aattaatggt actcctataa tttttgattg tgagctcacc
tatttgggtt aagcatgcca atttaaagag accaagtgta tgtacattat gttctacata
ttcagtgata aaattactaa actactatat gtctgcttta aatttgtact ttaatattgt
cttttggtat taagaaagat atgctttcag aatagatatg cttcgctttg gcaaggaatt
tggatagaac ttgctattta aaagaggtgt ggggtaaatc cttgtataaa tctccagttt
agcctttttt gaaaaagcta gactttcaaa tactaatttc acttcaagca gggtacgttt
ctggtttgtt tgcttgactt cagtcacaat ttcttatcag accaatggct gacctctttg
agatgtcagg ctaggcttac ctatgtgttc tgtgtcatgt gaatgctgag aagtttgaca
gagatccaac ttcagccttg accccatcag tccctcgggt taactaactg agccaccggt
cctcatggct attttaatga gggtattgat ggttaaatgc atgtctgatc ccttatccca
gccatttgca ctgccagctg ggaactatac cagacctgga tactgatccc aaagtgttaa
attcaactac atgctggaga ttagagatgg tgccaataaa ggacccagaa ccaggatctt
gattgctata gacttattaa taatccaggt caaagagagt gacacacact ctctcaagac
ctggggtgag ggagtctgtg ttatctgcaa ggccatttga ggctcagaaa gtctctcttt
cctatagata tatgcatact ttctgacata taggaatgta tcaggaatac tcaaccatca
caggcatgtt cctacctcag ggcctttaca tgtcctgttt actctgtcta gaatgtcctt
ctgtagatga cctggcttgc ctcgtcaccc ttcaggtcct tgctcaagtg tcatcttctc
ccctagttaa actaccccac accctgtctg ctttccttgc ttatttttct ccatagcatt
ttaccatctc ttacattaga catttttctt atttatttgt agtttataag cttcatgagg
caagtaactt tgctttgttt cttgctgtat ctccagtgcc cagagcagtg cctggtatat
aataaatatt tattgactga gtgaaaaaaa aaaaaaaaaa
CD70: The human CD70 sequence is represented by GenBank accession number
NM_001252.4 (SEQ ID NO: 14) listed below:
ccagagaggg gcaggctggt cccctgacag gttgaagcaa gtagacgccc aggagccccg
ggagggggct gcagtttcct tccttccttc tcggcagcgc tccgcgcccc catcgcccct
cctgcgctag cggaggtgat cgccgcggcg atgccggagg agggttcggg ctgctcggtg
cggcgcaggc cctatgggtg cgtcctgcgg gctgctttgg tcccattggt cgcgggcttg
gtgatctgcc tcgtggtgtg catccagcgc ttcgcacagg ctcagcagca gctgccgctc
gagtcacttg ggtgggacgt agctgagctg cagctgaatc acacaggacc tcagcaggac
cccaggctat actggcaggg gggcccagca ctgggccgct ccttcctgca tggaccagag
ctggacaagg ggcagctacg tatccatcgt gatggcatct acatggtaca catccaggtg
acgctggcca tctgctcctc cacgacggcc tccaggcacc accccaccac cctggccgtg
ggaatctgct ctcccgcctc ccgtagcatc agcctgctgc gtctcagctt ccaccaaggt
tgtaccattg cctcccagcg cctgacgccc ctggcccgag gggacacact ctgcaccaac
ctcactggga cacttttgcc ttcccgaaac actgatgaga ccttctttgg agtgcagtgg
gtgcgcccct gaccactgct gctgattagg gttttttaaa ttttatttta ttttatttaa
gttcaagaga aaaagtgtac acacaggggc cacccggggt tggggtggga gtgtggtggg
gggtagtggt ggcaggacaa gagaaggcat tgagcttttt ctttcatttt cctattaaaa
aatacaaaaa tca
CD27: The human CD27 sequence is represented by GenBank accession number
NM_001242.4 (SEQ ID NO: 15) listed below:
cggaagggga agggggtgga ggttgctgct atgagagaga aaaaaaaaac agccacaata
gagattctgc cttcaaaggt tggcttgcca cctgaagcag ccactgccca gggggtgcaa
agaagagaca gcagcgccca gcttggaggt gctaactcca gaggccagca tcagcaactg
ggcacagaaa ggagccgcct gggcagggac catggcacgg ccacatccct ggtggctgtg
cgttctgggg accctggtgg ggctctcagc tactccagcc cccaagagct gcccagagag
gcactactgg gctcagggaa agctgtgctg ccagatgtgt gagccaggaa cattcctcgt
gaaggactgt gaccagcata gaaaggctgc tcagtgtgat ccttgcatac cgggggtctc
cttctctcct gaccaccaca cccggcccca ctgtgagagc tgtcggcact gtaactctgg
tcttctcgtt cgcaactgca ccatcactgc caatgctgag tgtgcctgtc gcaatggctg
gcagtgcagg gacaaggagt gcaccgagtg tgatcctctt ccaaaccctt cgctgaccgc
tcggtcgtct caggccctga gcccacaccc tcagcccacc cacttacctt atgtcagtga
gatgctggag gccaggacag ctgggcacat gcagactctg gctgacttca ggcagctgcc
tgcccggact ctctctaccc actggccacc ccaaagatcc ctgtgcagct ccgattttat
tcgcatcctt gtgatcttct ctggaatgtt ccttgttttc accctggccg gggccctgtt
cctccatcaa cgaaggaaat atagatcaaa caaaggagaa agtcctgtgg agcctgcaga
gccttgtcgt tacagctgcc ccagggagga ggagggcagc accatcccca tccaggagga
ttaccgaaaa ccggagcctg cctgctcccc ctgagccagc acctgcggga gctgcactac
agccctggcc tccaccccca ccccgccgac catccaaggg agagtgagac ctggcagcca
caactgcagt cccatcctct tgtcagggcc ctttcctgtg tacacgtgac agagtgcctt
ttcgagactg gcagggacga ggacaaatat ggatgaggtg gagagtggga agcaggagcc
cagccagctg cgcctgcgct gcaggagggc gggggctctg gttgtaaaac acacttcctg
ctgcgaaaga cccacatgct acaagacggg caaaataaag tgacagatga ccaccctgca
RORgT: The human RORgT sequence is represented by GenBank accession number
NM_005060.3 (SEQ ID NO: 16) listed below:
gccaggtgct cccgccttcc accctccgcc ctcctccctc ccctgggccc tgctccctgc
cctcctgggc agccagggca gccaggacgg caccaaggga gctgccccat ggacagggcc
ccacagagac agcaccgagc ctcacgggag ctgctggctg caaagaagac ccacacctca
caaattgaag tgatcccttg caaaatctgt ggggacaagt cgtctgggat ccactacggg
gttatcacct gtgaggggtg caagggcttc ttccgccgga gccagcgctg taacgcggcc
tactcctgca cccgtcagca gaactgcccc atcgaccgca ccagccgaaa ccgatgccag
cactgccgcc tgcagaaatg cctggcgctg ggcatgtccc gagatgctgt caagttcggc
cgcatgtcca agaagcagag ggacagcctg catgcagaag tgcagaaaca gctgcagcag
cggcaacagc agcaacagga accagtggtc aagacccctc cagcaggggc ccaaggagca
gataccctca cctacacctt ggggctccca gacgggcagc tgcccctggg ctcctcgcct
gacctgcctg aggcttctgc ctgtccccct ggcctcctga aagcctcagg ctctgggccc
tcatattcca acaacttggc caaggcaggg ctcaatgggg cctcatgcca ccttgaatac
agccctgagc ggggcaaggc tgagggcaga gagagcttct atagcacagg cagccagctg
acccctgacc gatgtggact tcgttttgag gaacacaggc atcctgggct tggggaactg
ggacagggcc cagacagcta cggcagcccc agtttccgca gcacaccgga ggcaccctat
gcctccctga cagagataga gcacctggtg cagagcgtct gcaagtccta cagggagaca
tgccagctgc ggctggagga cctgctgcgg cagcgctcca acatcttctc ccgggaggaa
gtgactggct accagaggaa gtccatgtgg gagatgtggg aacggtgtgc ccaccacctc
accgaggcca ttcagtacgt ggtggagttc gccaagaggc tctcaggctt tatggagctc
tgccagaatg accagattgt gcttctcaaa gcaggagcaa tggaagtggt gctggttagg
atgtgccggg cctacaatgc tgacaaccgc acggtctttt ttgaaggcaa atacggtggc
atggagctgt tccgagcctt gggctgcagc gagctcatca gctccatctt tgacttctcc
cactccctaa gtgccttgca cttttccgag gatgagattg ccctctacac agcccttgtt
ctcatcaatg cccatcggcc agggctccaa gagaaaagga aagtagaaca gctgcagtac
aatctggagc tggcctttca tcatcatctc tgcaagactc atcgccaaag catcctggca
aagctgccac ccaaggggaa gcttcggagc ctgtgtagcc agcatgtgga aaggctgcag
atcttccagc acctccaccc catcgtggtc caagccgctt tccctccact ctacaaggag
ctcttcagca ctgaaaccga gtcacctgtg gggctgtcca agtgacctgg aagagggact
ccttgcctct ccctatggcc tgctggccca cctccctgga ccccgttcca ccctcaccct
tttcctttcc catgaaccct ggagggtggt ccccaccagc tctttggaag tgagcagatg
ctgcggctgg ctttctgtca gcaggccggc ctggcagtgg gacaatcgcc agagggtggg
gctggcagaa caccatctcc agcctcagct ttgacctgtc tcatttccca tattccttca
cacccagctt ctggaaggca tggggtggct gggatttaag gacttctggg ggaccaagac
atcctcaaga aaacaggggc atccagggct ccctggatga atagaatgca attcattcag
aagctcagaa gctaagaata agcctttgaa atacctcatt gcatttccct ttgggcttcg
gcttggggag atggatcaag ctcagagact ggcagtgaga gcccagaagg acctgtataa
aatgaatctg gagctttaca ttttctgcct ctgccttcct cccagctcag caaggaagta
tttgggcacc ctacccttta cctggggtct aaccaaaaat ggatgggatg aggatgagag
gctggagata attgttttat gggatttggg tgtgggacta gggtacaatg aaggccaaga
gcatctcaga catagagtta aaactcaaac ctcttatgtg cactttaaag atagacttta
ggggctggca caaatctgat cagagacaca tatccataca caggtgaaac acatacagac
tcaacagcaa tcatgcagtt ccagagacac atgaacctga cacaatctct cttatccttg
aggccacagc ttggaggagc ctagaggcct caggggaaag tcccaatcct gagggaccct
cccaaacatt tccatggtgc tccagtccac tgatcttggg tctggggtga tccaaatacc
accccagctc cagctgtctt ctaccactag aagacccaag agaagcagaa gtcgctcgca
ctggtcagtc ggaaggcaag atcagatcct ggaggacttt cctggcctgc ccgccagccc
tgctcttgtt gtggagaagg aagcagatgt gatcacatca ccccgtcatt gggcaccgct
gactccagca tggaggacac cagggagcag ggcctgggcc tgtttcccca gctgtgatct
gcccagaac ctctcttggc ttcataaaca gctgtgaacc ctcccctgag ggattaacag
caatgatggg cagtcgtgga gttggggggg ttgggggtgg gattgtgtcc tctaagggga
cgggttcatc tgagtaaaca taaaccccaa cttgtgccat tctttataaa atgattttaa
aggcaaaaaa aaaaaaaaaa aaaa
Tbx21: The human Tbx21 sequence is represented by GenBank accession number
NM_013351.1 (SEQ ID NO: 17) listed below:
cggcccgctg gagaggaagc ccgagagctg ccgcgcgcct gccggacgag ggcgtagaag
ccaggcgtca gagcccgggc tccggtgggg tcccccaccc ggccctcggg tcccccgccc
cctgctccct gcccatccca gcccacgcga ccctctcgcg cgcggagggg cgggtcctcg
acggctacgg gaaggtgcca gcccgccccg gatgggcatc gtggagccgg gttgcggaga
catgctgacg ggcaccgagc cgatgccggg gagcgacgag ggccgggcgc ctggcgccga
cccgcagcac cgctacttct acccggagcc gggcgcgcag gacgcggacg agcgtcgcgg
gggcggcagc ctggggtctc cctacccggg gggcgccttg gtgcccgccc cgccgagccg
cttccttgga gcctacgcct acccgccgcg accccaggcg gccggcttcc ccggcgcggg
cgagtccttc ccgccgcccg cggacgccga gggctaccag ccgggcgagg gctacgccgc
cccggacccg cgcgccgggc tctacccggg gccgcgtgag gactacgcgc tacccgcggg
actggaggtg tcggggaaac tgagggtcgc gctcaacaac cacctgttgt ggtccaagtt
taatcagcac cagacagaga tgatcatcac caagcaggga cggcggatgt tcccattcct
gtcatttact gtggccgggc tggagcccac cagccactac aggatgtttg tggacgtggt
cttggtggac cagcaccact ggcggtacca gagcggcaag tgggtgcagt gtggaaaggc
cgagggcagc atgccaggaa accgcctgta cgtccacccg gactccccca acacaggagc
gcactggatg cgccaggaag tttcatttgg gaaactaaag ctcacaaaca acaagggggc
gtccaacaat gtgacccaga tgattgtgct ccagtccctc cataagtacc agccccggct
gcatatcgtt gaggtgaacg acggagagcc agaggcagcc tgcaacgctt ccaacacgca
tatctttact ttccaagaaa cccagttcat tgccgtgact gcctaccaga atgccgagat
tactcagctg aaaattgata ataacccctt tgccaaagga ttccgggaga actttgagtc
catgtacaca tctgttgaca ccagcatccc ctccccgcct ggacccaact gtcaattcct
tgggggagat cactactctc ctctcctacc caaccagtat cctgttccca gccgcttcta
ccccgacctt cctggccagg cgaaggatgt ggttccccag gcttactggc tgggggcccc
ccgggaccac agctatgagg ctgagtttcg agcagtcagc atgaagcctg cattcttgcc
ctctgcccct gggcccacca tgtcctacta ccgaggccag gaggtcctgg cacctggagc
tggctggcct gtggcacccc agtaccctcc caagatgggc ccggccagct ggttccgccc
tatgcggact ctgcccatgg aacccggccc tggaggctca gagggacggg gaccagagga
ccagggtccc cccttggtgt ggactgagat tgcccccatc cggccggaat ccagtgattc
aggactgggc gaaggagact ctaagaggag gcgcgtgtcc ccctatcctt ccagtggtga
cagctcctcc cctgctgggg ccccttctcc ttttgataag gaagctgaag gacagtttta
taactatttt cccaactgag cagatgacat gatgaaagga acagaaacag tgttattagg
ttggaggaca ccgactaatt tgggaaacgg atgaaggact gagaaggccc ccgctccctc
tggcccttct ctgtttagta gttggttggg gaagtggggc tcaagaagga ttttggggtt
caccagatgc ttcctggccc acgatgaaac ctgagagggg tgtccccttg ccccatcctc
tgccctaact acagtcgttt acctggtgct gcgtcttgct tttggtttcc agctggagaa
aagaagacaa gaaagtcttg ggcatgaagg agctttttgc atctagtggg tgggaggggt
caggtgtggg acatgggagc aggagactcc actttcttcc tttgtacagt aactttcaac
cttttcgttg gcatgtgtgt taatccctga tccaaaaaga acaaatacac gtatgttata
accatcagcc cgccagggtc agggaaagga ctcacctgac tttggacagc tggcctgggc
tccccctgct caaacacagt ggggatcaga gaaaaggggc tggaaagggg ggaatggccc
acatctcaag aagcaagata ttgtttgtgg tggttgtgtg tgggtgtgtg ttttttcttt
ttctttcttt ttattttttt tgaatggggg aggctattta ttgtactgag agtggtgtct
ggatatattc cttttgtctt catcactttc tgaaaataaa cataaaactg ttaaaaaaaa
aaaaaaaaa
ULBP3: The human ULBP3 sequence is represented by GenBank accession number
NM_024518.1 (SEQ ID NO: 18) listed below:
atggcagcgg ccgccagccc cgcgatcctt ccgcgcctcg cgattcttcc gtacctgcta
ttcgactggt ccgggacggg gcgggccgac gctcactctc tctggtataa cttcaccatc
attcatttgc ccagacatgg gcaacagtgg tgtgaggtcc agagccaggt ggatcagaag
aattttctct cctatgactg tggcagtgac aaggtcttat ctatgggtca cctagaagag
cagctgtatg ccacagatgc ctggggaaaa caactggaaa tgctgagaga ggtggggcag
aggctcagac tggaactggc tgacactgag ctggaggatt tcacacccag tggacccctc
acgctgcagg tcaggatgtc ttgtgagtgt gaagccgatg gatacatccg tggatcttgg
cagttcagct tcgatggacg gaagttcctc ctctttgact caaacaacag aaagtggaca
gtggttcacg ctggagccag gcggatgaaa gagaagtggg agaaggatag cggactgacc
accttcttca agatggtctc aatgagagac tgcaagagct ggcttaggga cttcctgatg
cacaggaaga agaggctgga acccacagca ccacccacca tggccccagg cttagctcaa
cccaaagcca tagccaccac cctcagtccc tggagcttcc tcatcatcct ctgcttcatc
ctccctggca tctga
MICA: The human MICA sequence is represented by GenBank accession number
NM_001289152.1 (SEQ ID NO: 19) listed below:
gtatcatttc agtgaaggtc actccagtct ttcatggagg ccaaactaag ggtgtaaatt
aggatcctca ctgaagtggc gggaccctaa gaggcttttt cctggcccct tagttgtggg
ttttcctgcg ggcggcgcag ccggtttcca tcagaaccgc ccagaggcgg acgctgcctt
cctggggtga cggagcagca ggaagcgttt tcggatcctg gaatacgtgg gcggcccgtg
ggaggggctg aggcgcagtt tcctactcac ccggatccga atcctccgcg gtgctgtttc
aagagagccg gattccagat cacgctccag cccggactcg gaattcctgc cctgcgggtc
tgcattttca taacgggcag gtgtgagtgc cctgcagctg gagaccagaa gcctgaaggc
agctcggccc tccccagccc acagcgccgt tattccgttt ctatatcagt aaacacattt
cattttccgt agaccagggc ggggtgacgg gtgatcccag tcctcgcagt gaattccggg
cagcaaaatt caaaacacat gcggccaagg ccgggcacgg tggttcacgc ctgtaatccc
agcactttgg gaggtcgagg cgggcgatca cctgaggtcg ggagctcgag accaacctga
ccaacatggg gaaatcccgt ctctactaaa aatataaaat tagacgggct tggtggtgaa
tgcctgtaat cccagctagt cgggaggctg aggcaggaga atcgcttaaa ccttggaggc
ggaggttgcg gtgagccgag atcgcgccat tgcacttcag cctgggcaac aagagggaaa
actccgtcgc aaaaactttc gggggcggag cggagccccg ccctgggtta tgtaagcgac
cgcgctgggc cgtttctctt tcttttccgg accctgcagt ggcgcctaaa gtctgagaga
gggaagtcgc ctctgtgctc gtgagtgcat ggggtataag agccccacag tcttcgttat
aacctcacgg tgctgtcctg ggatggatct gtgcagtcag ggtttcttgc tgaggtacat
ctggatggtc agcccttcct gcgctatgac aggcagaaat gcagggcaaa gccccaggga
cagtgggcag aagatgtcct gggaaataag acatgggaca gagagaccag ggacttgaca
gggaacggaa aggacctcag gatgaccctg gctcatatca aggaccagaa agaaggcttg
cattccctcc aggagattag ggtctgtgag atccatgaag acaacagcac caggagctcc
cagcatttct actacgatgg ggagctcttc ctctcccaaa acctggagac tgaggaatgg
acagtgcccc agtcctccag agctcagacc ttggccatga acgtcaggaa tttcttgaag
gaagatgcca tgaagaccaa gacacactat cacgctatgc atgcagactg cctgcaggaa
ctacggcgat atctagaatc cggcgtagtc ctgaggagaa cagtgccccc catggtgaat
gtcacccgca gcgaggcctc agagggcaac atcaccgtga catgcagggc ttccagcttc
tatccccgga atatcatact gacctggcgt caggatgggg tatctttgag ccacgacacc
cagcagtggg gggatgtcct gcctgatggg aatggaacct accagacctg ggtggccacc
aggatttgcc gaggagagga gcagaggttc acctgctaca tggaacacag cgggaatcac
agcactcacc ctgtgccctc tgggaaagtg ctggtgcttc agagtcattg gcagacattc
catgtttctg ctgttgctgc tggctgctgc tatttttgtt attattattt tctatgtccg
ttgttgtaag aagaaaacat cagctgcaga gggtccagag ctcgtgagcc tgcaggtcct
ggatcaacac ccagttggga cgagtgacca cagggatgcc acacagctcg gatttcagcc
tctgatgtca gctcttgggt ccactggctc cactgagggc acctagactc tacagccagg
cggctggaat tgaattccct gcctggatct cacaagcact ttccctcttg gtgcctcagt
ttcctgacct atgaaacaga gaaaataaaa gcacttattt attgttgttg gaggctgcaa
aatgttagta gatatgaggc atttgcagct gtgccatatt aaaaaaaaaa aaaaaaaa
KLRK1 (mRNA sequence for NKG2D): The human KLRK1 sequence is represented by
GenBank accession number NM_007360.3 (SEQ ID NO: 20) listed below:
actaagtatc tccactttca attctagatc aggaactgag gacatatcta aattttctag
ttttatagaa ggcttttatc cacaagaatc aagatcttcc ctctctgagc aggaatcctt
tgtgcattga agactttaga ttcctctctg cggtagacgt gcacttataa gtatttgatg
gggtggattc gtggtcggag gtctcgacac agctgggaga tgagtgaatt tcataattat
aacttggatc tgaagaagag tgatttttca acacgatggc aaaagcaaag atgtccagta
gtcaaaagca aatgtagaga aaatgcatct ccattttttt tctgctgctt catcgctgta
gccatgggaa tccgtttcat tattatggta acaatatgga gtgctgtatt cctaaactca
ttattcaacc aagaagttca aattcccttg accgaaagtt actgtggccc atgtcctaaa
aactggatat gttacaaaaa taactgctac caattttttg atgagagtaa aaactggtat
gagagccagg cttcttgtat gtctcaaaat gccagccttc tgaaagtata cagcaaagag
gaccaggatt tacttaaact ggtgaagtca tatcattgga tgggactagt acacattcca
acaaatggat cttggcagtg ggaagatggc tccattctct cacccaacct actaacaata
attgaaatgc agaagggaga ctgtgcactc tatgcctcga gctttaaagg ctatatagaa
aactgttcaa ctccaaatac gtacatctgc atgcaaagga ctgtgtaaag atgatcaacc
atctcaataa aagccaggaa cagagaagag attacaccag cggtaacact gccaactgag
actaaaggaa acaaacaaaa acaggacaaa atgaccaaag actgtcagat ttcttagact
ccacaggacc aaaccataga acaatttcac tgcaaacatg catgattctc caagacaaaa
gaagagagat cctaaaggca attcagatat ccccaaggct gcctctccca ccacaagccc
agagtggatg ggctggggga ggggtgctgt tttaatttct aaaggtagga ccaacaccca
ggggatcagt gaaggaagag aaggccagca gatcactgag agtgcaaccc caccctccac
aggaaattgc ctcatgggca gggccacagc agagagacac agcatgggca gtgccttccc
tgcctgtggg ggtcatgctg ccacttttaa tgggtcctcc acccaacggg gtcagggagg
tggtgctgcc ccagtgggcc atgattatct taaaggcatt attctccagc cttaagtaag
atcttaggac gtttcctttg ctatgatttg tacttgcttg agtcccatga ctgtttctct
tcctctcttt cttccttttg gaatagtaat atccatccta tgtttgtccc actattgtat
tttggaagca cataacttgt ttggtttcac aggttcacag ttaagaagga attttgcctc
tgaataaata gaatcttgag tctcatgcaa aaaaaaaaaa aaaaaa
PRDX5: The human PRDX5 sequence is represented by GenBank accession number
NM_012094.4 (SEQ ID NO: 21) listed below:
cgcgcctgcg cagtggaggc ggcccaggcc cgccttccgc agggtgtcgc cgctgtgccg
ctagcggtgc cccgcctgct gcggtggcac cagccaggag gcggagtgga agtggccgtg
gggcgggtat gggactagct ggcgtgtgcg ccctgagacg ctcagcgggc tatatactcg
tcggtggggc cggcggtcag tctgcggcag cggcagcaag acggtgcagt gaaggagagt
gggcgtctgg cggggtccgc agtttcagca gagccgctgc agccatggcc ccaatcaagg
tgggagatgc catcccagca gtggaggtgt ttgaagggga gccagggaac aaggtgaacc
tggcagagct gttcaagggc aagaagggtg tgctgtttgg agttcctggg gccttcaccc
ctggatgttc caagacacac ctgccagggt ttgtggagca ggctgaggct ctgaaggcca
agggagtcca ggtggtggcc tgtctgagtg ttaatgatgc ctttgtgact ggcgagtggg
gccgagccca caaggcggaa ggcaaggttc ggctcctggc tgatcccact ggggcctttg
ggaaggagac agacttatta ctagatgatt cgctggtgtc catctttggg aatcgacgtc
tcaagaggtt ctccatggtg gtacaggatg gcatagtgaa ggccctgaat gtggaaccag
atggcacagg cctcacctgc agcctggcac ccaatatcat ctcacagctc tgaggccctg
ggccagatta cttcctccac ccctccctat ctcacctgcc cagccctgtg ctggggccct
gcaattggaa tgttggccag atttctgcaa taaacacttg tggtttgcgg ccatctcctt
ggttaaaaaa aaa
JAK1: The human JAK1 sequence is represented by GenBank accession number
NM_002227.2 (SEQ ID NO: 22) listed below:
tgcagacagt gcgggcctgc gcccagtccc ggctgtcctc gccgcgaccc ctcctcagcc
ctgggcgcgc gcacgctggg gccccgcggg gctggccgcc tagcgagcct gccggtcgac
cccagccagc gcagcgacgg ggcgctgcct ggcccaggcg cacacggaag tgcgcttctc
tgaagtagct ttggaaagta gagaagaaaa tccagtttgc ttcttggaga acactggaca
gctgaataaa tgcagtatct aaatataaaa gaggactgca atgccatggc tttctgtgct
aaaatgagga gctccaagaa gactgaggtg aacctggagg cccctgagcc aggggtggaa
gtgatcttct atctgtcgga cagggagccc ctccggctgg gcagtggaga gtacacagca
gaggaactgt gcatcagggc tgcacaggca tgccgtatct ctcctctttg tcacaacctc
tttgccctgt atgacgagaa caccaagctc tggtatgctc caaatcgcac catcaccgtt
gatgacaaga tgtccctccg gctccactac cggatgaggt tctatttcac caattggcat
ggaaccaacg acaatgagca gtcagtgtgg cgtcattctc caaagaagca gaaaaatggc
tacgagaaaa aaaagattcc agatgcaacc cctctccttg atgccagctc actggagtat
ctgtttgctc agggacagta tgatttggtg aaatgcctgg ctcctattcg agaccccaag
accgagcagg atggacatga tattgagaac gagtgtctag ggatggctgt cctggccatc
tcacactatg ccatgatgaa gaagatgcag ttgccagaac tgcccaagga catcagctac
aagcgatata ttccagaaac attgaataag tccatcagac agaggaacct tctcaccagg
atgcggataa ataatgtttt caaggatttc ctaaaggaat ttaacaacaa gaccatttgt
gacagcagcg tgtccacgca tgacctgaag gtgaaatact tggctacctt ggaaactttg
acaaaacatt acggtgctga aatatttgag acttccatgt tactgatttc atcagaaaat
gagatgaatt ggtttcattc gaatgacggt ggaaacgttc tctactacga agtgatggtg
actgggaatc ttggaatcca gtggaggcat aaaccaaatg ttgtttctgt tgaaaaggaa
aaaaataaac tgaagcggaa aaaactggaa aataaacaca agaaggatga ggagaaaaac
aagatccggg aagagtggaa caatttttct tacttccctg aaatcactca cattgtaata
aaggagtctg tggtcagcat taacaagcag gacaacaaga aaatggaact gaagctctct
tcccacgagg aggccttgtc ctttgtgtcc ctggtagatg gctacttccg gctcacagca
gatgcccatc attacctctg caccgacgtg gcccccccgt tgatcgtcca caacatacag
aatggctgtc atggtccaat ctgtacagaa tacgccatca ataaattgcg gcaagaagga
agcgaggagg ggatgtacgt gctgaggtgg agctgcaccg actttgacaa catcctcatg
accgtcacct gctttgagaa gtctgagcag gtgcagggtg cccagaagca gttcaagaac
tttcagatcg aggtgcagaa gggccgctac agtctgcacg gttcggaccg cagcttcccc
agcttgggag acctcatgag ccacctcaag aagcagatcc tgcgcacgga taacatcagc
ttcatgctaa aacgctgctg ccagcccaag ccccgagaaa tctccaacct gctggtggct
actaagaaag cccaggagtg gcagcccgtc taccccatga gccagctgag tttcgatcgg
atcctcaaga aggatctggt gcagggcgag caccttggga gaggcacgag aacacacatc
tattctggga ccctgatgga ttacaaggat gacgaaggaa cttctgaaga gaagaagata
aaagtgatcc tcaaagtctt agaccccagc cacagggata tttccctggc cttcttcgag
gcagccagca tgatgagaca ggtctcccac aaacacatcg tgtacctcta tggcgtctgt
gtccgcgacg tggagaatat catggtggaa gagtttgtgg aagggggtcc tctggatctc
ttcatgcacc ggaaaagcga tgtccttacc acaccatgga aattcaaagt tgccaaacag
ctggccagtg ccctgagcta cttggaggat aaagacctgg tccatggaaa tgtgtgtact
aaaaacctcc tcctggcccg tgagggcatc gacagtgagt gtggcccatt catcaagctc
agtgaccccg gcatccccat tacggtgctg tctaggcaag aatgcattga acgaatccca
tggattgctc ctgagtgtgt tgaggactcc aagaacctga gtgtggctgc tgacaagtgg
agctttggaa ccacgctctg ggaaatctgc tacaatggcg agatcccctt gaaagacaag
acgctgattg agaaagagag attctatgaa agccggtgca ggccagtgac accatcatgt
aaggagctgg ctgacctcat gacccgctgc atgaactatg accccaatca gaggcctttc
ttccgagcca tcatgagaga cattaataag cttgaagagc agaatccaga tattgtttca
gaaaaaaaac cagcaactga agtggacccc acacattttg aaaagcgctt cctaaagagg
atccgtgact tgggagaggg ccactttggg aaggttgagc tctgcaggta tgaccccgaa
ggggacaata caggggagca ggtggctgtt aaatctctga agcctgagag tggaggtaac
cacatagctg atctgaaaaa ggaaatcgag atcttaagga acctctatca tgagaacatt
gtgaagtaca aaggaatctg cacagaagac ggaggaaatg gtattaagct catcatggaa
tttctgcctt cgggaagcct taaggaatat cttccaaaga ataagaacaa aataaacctc
aaacagcagc taaaatatgc cgttcagatt tgtaagggga tggactattt gggttctcgg
caatacgttc accgggactt ggcagcaaga aatgtccttg ttgagagtga acaccaagtg
aaaattggag acttcggttt aaccaaagca attgaaaccg ataaggagta ttacaccgtc
aaggatgacc gggacagccc tgtgttttgg tatgctccag aatgtttaat gcaatctaaa
ttttatattg cctctgacgt ctggtctttt ggagtcactc tgcatgagct gctgacttac
tgtgattcag attctagtcc catggctttg ttcctgaaaa tgataggccc aacccatggc
cagatgacag tcacaagact tgtgaatacg ttaaaagaag gaaaacgcct gccgtgccca
cctaactgtc cagatgaggt ttatcaactt atgaggaaat gctgggaatt ccaaccatcc
aatcggacaa gctttcagaa ccttattgaa ggatttgaag cacttttaaa ataagaagca
tgaataacat ttaaattcca cagattatca agtccttctc ctgcaacaaa tgcccaagtc
attttttaaa aatttctaat gaaagaagtt tgtgttctgt ccaaaaagtc actgaactca
tacttcagta catatacatg tataaggcac actgtagtgc ttaatatgtg taaggacttc
ctctttaaat ttggtaccag taacttagtg acacataatg acaaccaaaa tatttgaaag
cacttaagca ctcctccttg tggaaagaat ataccaccat ttcatctggc tagttcacca
tcacaactgc attaccaaaa ggggattttt gaaaacgagg agttgaccaa aataatatct
gaagatgatt gcttttccct gctgccagct gatctgaaat gttttgctgg cacattaatc
atagataaag aaagattgat ggacttagcc ctcaaatttc agtatctata cagtactaga
ccatgcattc ttaaaatatt agataccagg tagtatatat tgtttctgta caaaaatgac
tgtattctct caccagtagg acttaaactt tgtttctcca gtggcttagc tcctgttcct
ttgggtgatc actagcaccc atttttgaga aagctggttc tacatggggg gatagctgtg
gaatagataa tttgctgcat gttaattctc aagaactaag cctgtgccag tgctttccta
agcagtatac ctttaatcag aactcattcc cagaacctgg atgctattac acatgctttt
aagaaacgtc aatgtatatc cttttataac tctaccactt tggggcaagc tattccagca
ctggttttga atgctgtatg caaccagtct gaataccaca tacgctgcac tgttcttaga
gggtttccat acttaccacc gatctacaag ggttgatccc tgtttttacc atcaatcatc
accctgtggt gcaacacttg aaagacccgg ctagaggcac tatggacttc aggatccact
agacagtttt cagtttgctt ggaggtagct gggtaatcaa aaatgtttag tcattgattc
aatgtgaacg attacggtct ttatgaccaa gagtctgaaa atctttttgt tatgctgttt
agtattcgtt tgatattgtt acttttcacc tgttgagccc aaattcagga ttggttcagt
ggcagcaatg aagttgccat ttaaatttgt tcatagccta catcaccaag gtctctgtgt
caaacctgtg gccactctat atgcactttg tttactcttt atacaaataa atatactaaa
gactttacat gca
JAK2: The human JAK2 sequence is represented by GenBank accession number
NM_004972.3 (SEQ ID NO: 23) listed below:
ctgcaggaag gagagaggaa gaggagcaga agggggcagc agcggacgcc gctaacggcc
tccctcggcg ctgacaggct gggccggcgc ccggctcgct tgggtgttcg cgtcgccact
tcggcttctc ggccggtcgg gcccctcggc ccgggcttgc ggcgcgcgtc ggggctgagg
gctgctgcgg cgcagggaga ggcctggtcc tcgctgccga gggatgtgag tgggagctga
gcccacactg gagggccccc gagggcccag cctggaggtc gttcagagcc gtgcccgtcc
cggggcttcg cagaccttga cccgccgggt aggagccgcc cctgcgggct cgagggcgcg
ctctggtcgc ccgatctgtg tagccggttt cagaagcagg caacaggaac aagatgtgaa
ctgtttctct tctgcagaaa aagaggctct tcctcctcct cccgcgacgg caaatgttct
gaaaaagact ctgcatggga atggcctgcc ttacgatgac agaaatggag ggaacatcca
cctcttctat atatcagaat ggtgatattt ctggaaatgc caattctatg aagcaaatag
atccagttct tcaggtgtat ctttaccatt cccttgggaa atctgaggca gattatctga
cctttccatc tggggagtat gttgcagaag aaatctgtat tgctgcttct aaagcttgtg
gtatcacacc tgtgtatcat aatatgtttg ctttaatgag tgaaacagaa aggatctggt
atccacccaa ccatgtcttc catatagatg agtcaaccag gcataatgta ctctacagaa
taagatttta ctttcctcgt tggtattgca gtggcagcaa cagagcctat cggcatggaa
tatctcgagg tgctgaagct cctcttcttg atgactttgt catgtcttac ctctttgctc
agtggcggca tgattttgtg cacggatgga taaaagtacc tgtgactcat gaaacacagg
aagaatgtct tgggatggca gtgttagata tgatgagaat agccaaagaa aacgatcaaa
ccccactggc catctataac tctatcagct acaagacatt cttaccaaaa tgtattcgag
caaagatcca agactatcat attttgacaa ggaagcgaat aaggtacaga tttcgcagat
ttattcagca attcagccaa tgcaaagcca ctgccagaaa cttgaaactt aagtatctta
taaatctgga aactctgcag tctgccttct acacagagaa atttgaagta aaagaacctg
gaagtggtcc ttcaggtgag gagatttttg caaccattat aataactgga aacggtggaa
ttcagtggtc aagagggaaa cataaagaaa gtgagacact gacagaacag gatttacagt
tatattgcga ttttcctaat attattgatg tcagtattaa gcaagcaaac caagagggtt
caaatgaaag ccgagttgta actatccata agcaagatgg taaaaatctg gaaattgaac
ttagctcatt aagggaagct ttgtctttcg tgtcattaat tgatggatat tatagattaa
ctgcagatgc acatcattac ctctgtaaag aagtagcacc tccagccgtg cttgaaaata
tacaaagcaa ctgtcatggc ccaatttcga tggattttgc cattagtaaa ctgaagaaag
caggtaatca gactggactg tatgtacttc gatgcagtcc taaggacttt aataaatatt
ttttgacttt tgctgtcgag cgagaaaatg tcattgaata taaacactgt ttgattacaa
aaaatgagaa tgaagagtac aacctcagtg ggacaaagaa gaacttcagc agtcttaaag
atcttttgaa ttgttaccag atggaaactg ttcgctcaga caatataatt ttccagttta
ctaaatgctg tcccccaaag ccaaaagata aatcaaacct tctagtcttc agaacgaatg
gtgtttctga tgtaccaacc tcaccaacat tacagaggcc tactcatatg aaccaaatgg
tgtttcacaa aatcagaaat gaagatttga tatttaatga aagccttggc caaggcactt
ttacaaagat ttttaaaggc gtacgaagag aagtaggaga ctacggtcaa ctgcatgaaa
cagaagttct tttaaaagtt ctggataaag cacacagaaa ctattcagag tctttctttg
aagcagcaag tatgatgagc aagctttctc acaagcattt ggttttaaat tatggagtat
gtgtctgtgg agacgagaat attctggttc aggagtttgt aaaatttgga tcactagata
catatctgaa aaagaataaa aattgtataa atatattatg gaaacttgaa gttgctaaac
agttggcatg ggccatgcat tttctagaag aaaacaccct tattcatggg aatgtatgtg
ccaaaaatat tctgcttatc agagaagaag acaggaagac aggaaatcct cctttcatca
aacttagtga tcctggcatt agtattacag ttttgccaaa ggacattctt caggagagaa
taccatgggt accacctgaa tgcattgaaa atcctaaaaa tttaaatttg gcaacagaca
aatggagttt tggtaccact ttgtgggaaa tctgcagtgg aggagataaa cctctaagtg
ctctggattc tcaaagaaag ctacaatttt atgaagatag gcatcagctt cctgcaccaa
agtgggcaga attagcaaac cttataaata attgtatgga ttatgaacca gatttcaggc
cttctttcag agccatcata cgagatctta acagtttgtt tactccagat tatgaactat
taacagaaaa tgacatgtta ccaaatatga ggataggtgc cctggggttt tctggtgcct
ttgaagaccg ggatcctaca cagtttgaag agagacattt gaaatttcta cagcaacttg
gcaagggtaa ttttgggagt gtggagatgt gccggtatga ccctctacag gacaacactg
gggaggtggt cgctgtaaaa aagcttcagc atagtactga agagcaccta agagactttg
aaagggaaat tgaaatcctg aaatccctac agcatgacaa cattgtaaag tacaagggag
tgtgctacag tgctggtcgg cgtaatctaa aattaattat ggaatattta ccatatggaa
gtttacgaga ctatcttcaa aaacataaag aacggataga tcacataaaa cttctgcagt
acacatctca gatatgcaag ggtatggagt atcttggtac aaaaaggtat atccacaggg
atctggcaac gagaaatata ttggtggaga acgagaacag agttaaaatt ggagattttg
ggttaaccaa agtcttgcca caagacaaag aatactataa agtaaaagaa cctggtgaaa
gtcccatatt ctggtatgct ccagaatcac tgacagagag caagttttct gtggcctcag
atgtttggag ctttggagtg gttctgtatg aacttttcac atacattgag aagagtaaaa
gtccaccagc ggaatttatg cgtatgattg gcaatgacaa acaaggacag atgatcgtgt
tccatttgat agaacttttg aagaataatg gaagattacc aagaccagat ggatgcccag
atgagatcta tatgatcatg acagaatgct ggaacaataa tgtaaatcaa cgcccctcct
ttagggatct agctcttcga gtggatcaaa taagggataa catggctgga tgaaagaaat
gaccttcatt ctgagaccaa agtagattta cagaacaaag ttttatattt cacattgctg
tggactatta ttacatatat cattattata taaatcatga tgctagccag caaagatgtg
aaaatatctg ctcaaaactt tcaaagttta gtaagttttt cttcatgagg ccaccagtaa
aagacattaa tgagaattcc ttagcaagga ttttgtaaga agtttcttaa acattgtcag
ttaacatcac tcttgtctgg caaaagaaaa aaaatagact ttttcaactc agctttttga
gacctgaaaa aattattatg taaattttgc aatgttaaag atgcacagaa tatgtatgta
tagtttttac cacagtggat gtataatacc ttggcatctt gtgtgatgtt ttacacacat
gagggctggt gttcattaat actgttttct aatttttcca tagttaatct ataattaatt
acttcactat acaaacaaat taagatgttc agataattga ataagtacct ttgtgtcctt
gttcatttat atcgctggcc agcattataa gcaggtgtat acttttagct tgtagttcca
tgtactgtaa atatttttca cataaaggga acaaatgtct agttttattt gtataggaaa
tttccctgac cctaaataat acattttgaa atgaaacaag cttacaaaga tataatctat
tttattatgg tttcccttgt atctatttgt ggtgaatgtg ttttttaaat ggaactatct
ccaaattttt ctaagactac tatgaacagt tttcttttaa aattttgaga ttaagaatgc
caggaatatt gtcatccttt gagctgctga ctgccaataa cattcttcga tctctgggat
ttatgctcat gaactaaatt taagcttaag ccataaaata gattagattg ttttttaaaa
atggatagct cattaagaag tgcagcaggt taagaatttt ttcctaaaga ctgtatattt
gaggggtttc agaattttgc attgcagtca tagaagagat ttatttcctt tttagagggg
aaatgaggta aataagtaaa aaagtatgct tgttaatttt attcaagaat gccagtagaa
aattcataac gtgtatcttt aagaaaaatg agcatacatc ttaaatcttt tcaattaagt
ataaggggtt gttcgttgtt gtcatttgtt atagtgctac tccactttag acaccatagc
taaaataaaa tatggtgggt tttgtgtgtg tgtgtgtgtg tgtgtgtgtg tgtgtgtgtg
tgttatttat acaaaactta aaatacttgc tgttttgatt aaaaagaaaa tagtttctta
cttta
CTGF: The human CTGF sequence is represented by GenBank accession number
NM_001901.2 (SEQ ID NO: 24) listed below:
aaactcacac aacaactctt ccccgctgag aggagacagc cagtgcgact ccaccctcca
gctcgacggc agccgccccg gccgacagcc ccgagacgac agcccggcgc gtcccggtcc
ccacctccga ccaccgccag cgctccaggc cccgccgctc cccgctcgcc gccaccgcgc
cctccgctcc gcccgcagtg ccaaccatga ccgccgccag tatgggcccc gtccgcgtcg
ccttcgtggt cctcctcgcc ctctgcagcc ggccggccgt cggccagaac tgcagcgggc
cgtgccggtg cccggacgag ccggcgccgc gctgcccggc gggcgtgagc ctcgtgctgg
acggctgcgg ctgctgccgc gtctgcgcca agcagctggg cgagctgtgc accgagcgcg
acccctgcga cccgcacaag ggcctcttct gtgacttcgg ctccccggcc aaccgcaaga
tcggcgtgtg caccgccaaa gatggtgctc cctgcatctt cggtggtacg gtgtaccgca
gcggagagtc cttccagagc agctgcaagt accagtgcac gtgcctggac ggggcggtgg
gctgcatgcc cctgtgcagc atggacgttc gtctgcccag ccctgactgc cccttcccga
ggagggtcaa gctgcccggg aaatgctgcg aggagtgggt gtgtgacgag cccaaggacc
aaaccgtggt tgggcctgcc ctcgcggctt accgactgga agacacgttt ggcccagacc
caactatgat tagagccaac tgcctggtcc agaccacaga gtggagcgcc tgttccaaga
cctgtgggat gggcatctcc acccgggtta ccaatgacaa cgcctcctgc aggctagaga
agcagagccg cctgtgcatg gtcaggcctt gcgaagctga cctggaagag aacattaaga
agggcaaaaa gtgcatccgt actcccaaaa tctccaagcc tatcaagttt gagctttctg
gctgcaccag catgaagaca taccgagcta aattctgtgg agtatgtacc gacggccgat
gctgcacccc ccacagaacc accaccctgc cggtggagtt caagtgccct gacggcgagg
tcatgaagaa gaacatgatg ttcatcaaga cctgtgcctg ccattacaac tgtcccggag
acaatgacat ctttgaatcg ctgtactaca ggaagatgta cggagacatg gcatgaagcc
agagagtgag agacattaac tcattagact ggaacttgaa ctgattcaca tctcattttt
ccgtaaaaat gatttcagta gcacaagtta tttaaatctg tttttctaac tgggggaaaa
gattcccacc caattcaaaa cattgtgcca tgtcaaacaa atagtctatc aaccccagac
actggtttga agaatgttaa gacttgacag tggaactaca ttagtacaca gcaccagaat
gtatattaag gtgtggcttt aggagcagtg ggagggtacc agcagaaagg ttagtatcat
cagatagcat cttatacgag taatatgcct gctatttgaa gtgtaattga gaaggaaaat
tttagcgtgc tcactgacct gcctgtagcc ccagtgacag ctaggatgtg cattctccag
ccatcaagag actgagtcaa gttgttcctt aagtcagaac agcagactca gctctgacat
tctgattcga atgacactgt tcaggaatcg gaatcctgtc gattagactg gacagcttgt
ggcaagtgaa tttgcctgta acaagccaga ttttttaaaa tttatattgt aaatattgtg
tgtgtgtgtg tgtgtgtata tatatatata tgtacagtta tctaagttaa tttaaagttg
tttgtgcctt tttatttttg tttttaatgc tttgatattt caatgttagc ctcaatttct
gaacaccata ggtagaatgt aaagcttgtc tgatcgttca aagcatgaaa tggatactta
tatggaaatt ctgctcagat agaatgacag tccgtcaaaa cagattgttt gcaaagggga
ggcatcagtg tccttggcag gctgatttct aggtaggaaa tgtggtagcc tcacttttaa
tgaacaaatg gcctttatta aaaactgagt gactctatat agctgatcag ttttttcacc
tggaagcatt tgtttctact ttgatatgac tgtttttcgg acagtttatt tgttgagagt
gtgaccaaaa gttacatgtt tgcacctttc tagttgaaaa taaagtgtat attttttcta
taaaaaaaaa aaaaaaaa

### Example 3: Compatibility of DPCP with Solvents for Gels and Ointments

The compatibility and solubility of DPCP was determined in both isopropyl myristate (IPM) as well as Polysorbate 80 (PS80). The solubility of DPCP in IPM is ˜1.1% w/w and DPCP was found to be highly soluble in PS80. Next, the stability of DPCP in these solvents was determined. A solution of 0.4% DPCP in isopropyl myristate and a solution of 0.4% DPCP in Polysorbate 80 was placed at 50° C. for two weeks. The stability of DPCP in these solvents was determined using reverse phase HPLC on a C18 column.

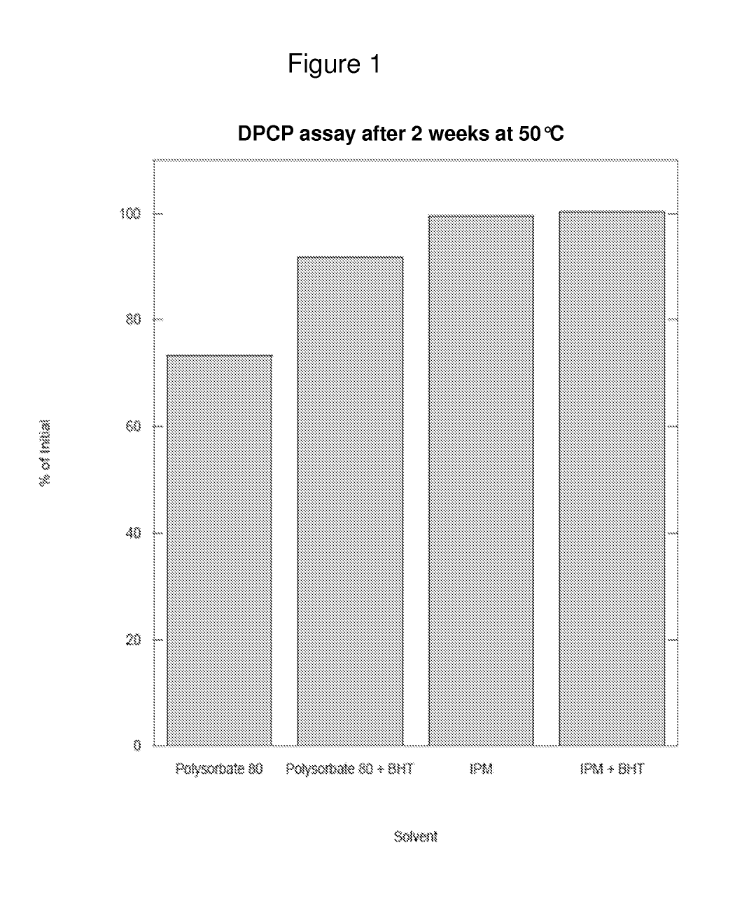

DPCP is stable in IPM at accelerated conditions; however, some degradation of DPCP was observed in the presence of PS80 (FIG. 1). Butylated hydroxytoloune (BHT) was shown to reduce the amount of degradation of DPCP in PS80.

### Example 4: Stability of DPCP in Ethanol and Isopropyl Alcohol

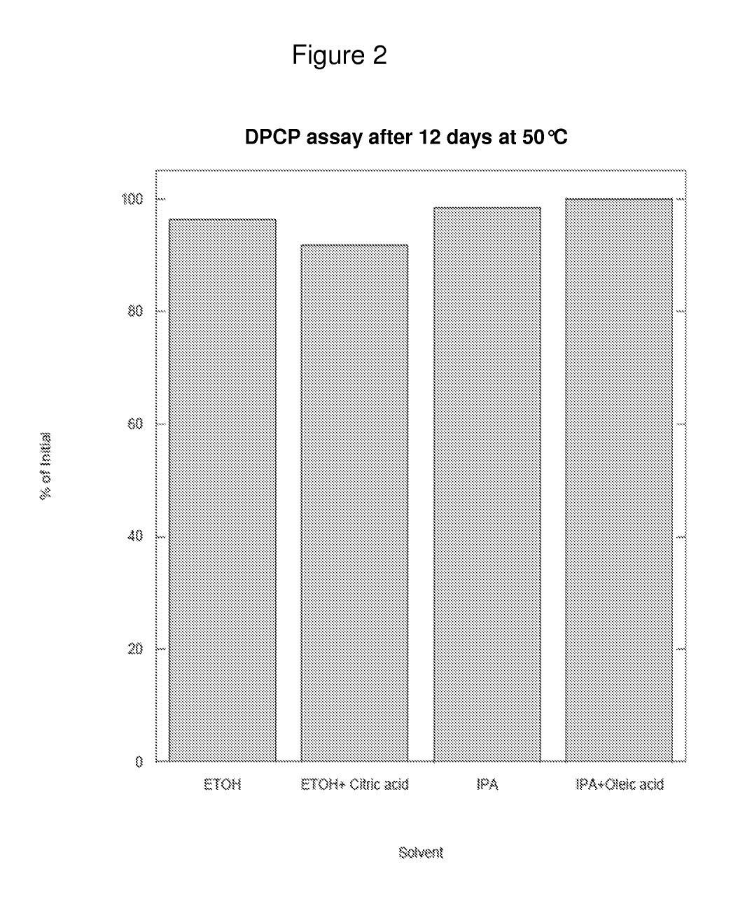

For the development of a gel formulation, the stability of DPCP was determined in both Ethanol (ETOH) and Isopropanol (IPA) (0.4% DPCP solutions in each solvent was placed at 50° C. for two weeks; FIG. 2. All solutions contained 0.1% BHT). Some degradation of DPCP in ETOH was observed in the presence of citric acid. However, DPCP was stable in IPA. The stability of DPCP in the above solvents was determined using reverse phase HPLC on a C18 column.

### Example 5: Ointment Formulations (Ointment 1)

Ointments containing haptens can comprise one or more of the following excipients:

| Excipient | % w/w |
|---|---|
| BHT | 0.1% |
| Methylparaben | 0.1% |
| Propylparaben | 0.05% |
| Cetyl esters wax | 10% |
| White wax | 10% |
| Polysorbate 80^{a} | 39.875% |
| Isopropyl myristate^{a} | 39.875% |

^{a}These excipients can be reduced slightly in formulations containing DPCP

### Example 6: Further Ointment Formulations (Ointment 2)

Ointments containing haptens can comprise one or more of the following excipients:

| Excipient | % w/w |
|---|---|
| BHT | 0.1% |
| Methylparaben | 0.1% |
| Propylparaben | 0.05% |
| Glyceryl monostearate, EP | 5% |
| Cetyl esters wax | 7.5% |
| White wax | 7.5% |
| Polysorbate 80^{a} | 39.875% |
| Isopropyl myristate^{a} | 39.875% |

^{a}These excipients can be reduced slightly in formulations containing DPCP

### Example 7: Gel Formulations

Gels containing haptens can comprise one or more of the following excipients:

| Excipient | % w/w |
|---|---|
| BHT | 0.1% |
| Klucel ME Pharm | 2% |
| Isopropyl alcohol | 57.9% |
| Propylene glycol | 10% |
| Polysorbate 80^{a} | 15% |
| Isopropyl myristate^{a} | 15% |

^{a}These excipients can be reduced slightly in formulations containing DPCP

### Example 8: Stability of Formulations at 3 Weeks

The gel and ointment formulations outlined in Examples 5-7 (containing 0.4% DPCP), were manufactured and their stability was monitored over a period of 3 weeks at both 25° C. and 30° C. The appearance, strength of DPCP and viscosity of the formulations were observed. At the 3 week time point, no significant changes were observed at the 25° C. or 30° C. conditions.

TABLE 1
Initial Test Results

| | | Assay, | Viscosity, |
|---|---|---|---|
| Formulation | Appearance | % w/w | cP |
| Ointment 1 | Off-white to beige | 0.386 | 23,000¹ |
| (0.4% DPCP) | homogeneous ointment | | |
| Ointment 2 | Off-white to beige | 0.392 | 24,500¹ |
| (0.4% DPCP) | homogeneous ointment | | |
| Gel | Clear to translucent | 0.388 | 89,000² |
| (0.4% DPCP) | slightly granular gel | | |

¹Rheosys cone/plate, 1 rpm, 20° C.
²Brookfield LV, spindle #14, sample holder #6R, 20° C.

TABLE 2
Assay and Viscosity after 3 weeks at 30° C.:

| | | Assay, | Viscosity, |
|---|---|---|---|
| Formulation | Appearance | % initial | cP |
| Ointment 1 | Very slightly softened | 99.2 | 22,200¹ |
| (0.4% DPCP) | with no syneresis | | |
| Ointment 2 | Slightly softened with | 98.7 | 24,000¹ |
| (0.4% DPCP) | no syneresis | | |
| Gel | Clear to translucent | 101.2 | 91,000² |
| (0.4% DPCP) | slightly granular gel | | |

¹Rheosys cone/plate, 1 rpm, 20° C.
²Brookfield LV, spindle #14, sample holder #6R, 20° C.

TABLE 3
Appearance after 3 weeks:

| Formulation | 25° C. | 30° C. | 40° C. |
|---|---|---|---|
| Ointment 1 | Off-white | Very slightly | Very soft, |
| (0.4% DPCP) | to beige | softened with | pourable with |
| | homogeneous | no syneresis | no syneresis |
| | ointment | | |
| Ointment 2 | Off-white | Slightly | Liquefied, |
| (0.4% DPCP) | to beige | softened with | with some |
| | homogeneous | no syneresis | very slight |
| | ointment | | syneresis |
| Gel | Clear to | Clear to | Clear to |
| (0.4% DPCP) | translucent | translucent | translucent |
| | slightly | slightly | slightly |
| | granular gel | granular gel | granular gel |

In any of the embodiments discussed above, a hapten, such as DPCP can be topically administered as a gel, ointment or cream. Sensitization dose (in the range of 0.1% DPCP to 1% DPCP) can be provided approximately 2 weeks prior to challenge dose. Challenge dose (in the range of 0.0000001% to 0.4% DPCP) can be provided approximately two weeks post sensitization dose and then approximately twice every week, once every week, once every two weeks or once every three weeks. In case of a relapse, dosing can be re-initiated.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method for treating alopecia areata comprising administering to a subject in need thereof a therapeutically effective amount of a hapten that reduces the expression of a gene encoding and/or a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2RΦ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

2. The method of claim 1, wherein the hapten is DPCP, imiquimod, ingenol mebutate, or SADBE.

3. The method of claim 1 or 2, wherein the hapten is DPCP.

4. The method of claim 3, wherein a therapeutically effective amount of DPCP is used to reduce levels of Tbx21 for treating alopecia areata.

5. The method of any one of claims 1 to 4, wherein the hapten is formulated in a composition comprising a gel formulation.

6. The method of claim 5, wherein a low sensitizing dose of the composition is administered to a first site on the skin of the subject, followed by a subsequent administration of a challenge dose of the composition to a second site on the skin of the subject, wherein the composition comprises DPCP.

7. The method of claim 6, wherein the low sensitizing dose is about 0.1 to about 1% DPCP, and wherein the challenge dose is 0.0000001% to about 0.4% DPCP.

8. The method of claim 6, wherein the sensitizing dose is 0.4% DPCP.

9. The method of claim 6, wherein the challenge dose is administered to the skin daily.

10. The method of claim 6, wherein the challenge dose is administered to the skin every other day.

11. The method of claim 6, wherein the challenge dose is administered to the skin twice a week.

12. The method of claim 6, wherein the challenge dose is administered to the skin weekly.

13. The method of claim 6, wherein the challenge dose is administered to the skin every two weeks.

14. The method of claim 6, wherein the challenge dose is administered to the skin every three weeks.

15. The method of claim 6, wherein the challenge dose is administered to the skin in any combination of daily, twice a week, weekly, every other week, every three weeks and/or monthly.

16. The method of claim 5, wherein the composition comprises DPCP.

17. The method of any one of claims 5 to 16, wherein the composition comprises a) a first co-solvent comprising a non-ionic surfactant; b) a second co-solvent comprising an alcoholic ester; and, c) a gelling agent.

18. The method of claim 17, wherein the first co-solvent is selected from the group consisting of polyoxyethylene (20) monoleate, polyoxyethylene (20) sorbitan monooleate, polysorbate 80, palmitate and stearate, wherein the second co-solvent is selected from the group consisting of isopropyl myristate and isopropyl palmitate, and wherein the gelling agent is selected from the group consisting of polyoxyl 40 stearate and hydroxypropyl cellulose.

19. A method for treating alopecia areata comprising administering to a subject in need thereof a therapeutically effective amount of at least one nucleic acid molecule that is directed against a gene encoding a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

20. The method of claim 19, wherein the nucleic acid molecule is a chemically modified oligonucleotide.

21. The method of claim 19 or 20, wherein the nucleic acid molecule is a double stranded nucleic acid molecule.

22. The method of claim 21, wherein the nucleic acid molecule is an isolated double stranded nucleic acid molecule that includes a double stranded region and a single stranded region, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the isolated double stranded nucleic acid molecule are modified.

23. The method of claim 22, wherein the isolated double stranded nucleic acid molecule further comprises a hydrophobic conjugate that is attached to the isolated double stranded nucleic acid molecule.

24. The method of any one of claims 19-23, wherein the nucleic acid molecule is directed against a gene encoding Tbx21.

25. The method of any one of claims 19-23, wherein the nucleic acid molecule is directed against a gene encoding CTGF.

26. The method of any one of claims 19-25, wherein the nucleic acid molecule silences gene expression through an RNAi mechanism of action.

27. The method of any one of claims 19-26, wherein the nucleic acid molecule is in a composition formulated for topical delivery.

28. The method of any one of claims 19-27, wherein the nucleic acid molecule is in a composition formulated for delivery to the skin.

29. The method of claim 28, wherein the nucleic acid molecule is in a composition formulated for intradermal injection.

30. The method of claim 28 or 29, wherein the nucleic acid molecule is in a composition formulated for extended release of the molecule following intradermal injection.

31. The method of any one of claims 19-30, wherein two or more nucleic acid molecules directed against genes encoding different proteins are administered to the subject.

32. The method of any one of claims 19-31, wherein two or more nucleic acid molecules directed against genes encoding the same protein are administered to the subject.

33. The method of any one of claims 19-32, wherein the nucleic acid molecule is composed of nucleotides and at least 30% of the nucleotides are chemically modified.

34. The method of any one of claims 19-33, wherein the nucleic acid molecule contains at least one modified backbone linkage.

35. The method of claim 34, wherein the nucleic acid molecule contains at least one phosphorothioate linkage.

36. The method of any one of claims 19-35, wherein the nucleic acid molecule is composed of nucleotides and at least one of the nucleotides contains a 2′ chemical modification selected from the group consisting of 2′OMe and 2′Fluoro.

37. The method of any one of claims 19-36, wherein the nucleic acid molecule is administered once.

38. The method of any one of claims 19-36, wherein the nucleic acid molecule is administered more than once.

39. The method of claim 24, wherein the nucleic acid molecule comprises at least 12 contiguous nucleotides of a sequence as set forth in SEQ ID NO.: 17.

40. The method of claim 25, wherein the nucleic acid molecule is directed against at least 12 contiguous nucleotides of a sequence as set forth in SEQ ID NO.: 24.

41. A method for treating alopecia areata comprising administering to a subject in need thereof a therapeutically effective amount of a hapten that reduces the expression of a gene encoding and/or a protein selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF and
a therapeutically effective amount of at least one nucleic acid molecule that is directed against a gene encoding a molecule selected from the group consisting of Interleukin 2 (IL-2), Interleukin 2 receptor (IL-2Rα or IL-2Rβ), Interleukin 15 (IL-15), Interleukin 15 receptor (IL15Rα, IL-2Rα or IL-2Rβ), Interleukin 12 (IL-12α or IL-12β), Interleukin 2 receptor (IL-12Rβ1 or IL-12Rβ2), Interleukin 17a (IL-17a), IFN-gamma (IFN-γ), CD28, CD70, CD27, RORγT, Tbx21, ULBP3, major histocompatibility complex class 1 polypeptide-related sequence A (MICA), NKG2d (KLRK1), PRDX5, JAK1, JAK2 and CTGF.

42. The method of claim 41, wherein the hapten is DPCP, imiquimod, ingenol mebutate, or SADBE.

43. The method of claim 41 or 42, wherein the hapten and the nucleic acid are administered separately.

44. The method of claim 41 or 42, wherein the hapten and the nucleic acid are administered at the same time.

45. The method of claim 41 or 42, wherein the hapten and the nucleic acid are administered in the same formulation.

46. The method of claim 41 or 42, wherein the administration of the hapten and the nucleic acid is temporally separate.

47. The method of any one of claims 1 to 4, wherein the hapten is formulated in a composition comprising an ointment formulation.

48. The method of claim 47, wherein a low sensitizing dose of the composition is administered to a first site on the skin of the subject, followed by a subsequent administration of a challenge dose of the composition to a second site on the skin of the subject, wherein the composition comprises DPCP.

49. The method of claim 48, wherein the low sensitizing dose is about 0.1 to about 1% DPCP, and wherein the challenge dose is 0.0000001% to about 0.4% DPCP.

50. The method of claim 48, wherein the sensitizing dose is 0.4% DPCP.

51. The method of claim 48, wherein the challenge dose is administered to the skin daily.

52. The method of claim 48, wherein the challenge dose is administered to the skin every other day.

53. The method of claim 48, wherein the challenge dose is administered to the skin twice a week.

54. The method of claim 48, wherein the challenge dose is administered to the skin weekly.

55. The method of claim 48, wherein the challenge dose is administered to the skin every two weeks.

56. The method of claim 48, wherein the challenge dose is administered to the skin every three weeks.

57. The method of claim 48, wherein said challenge dose is administered to the skin in any combination of daily, twice a week, weekly, every other week, every three weeks and/or monthly.

58. The method of claim 47, wherein the composition comprises DPCP.

59. The method of any one of claims 47-58, wherein the composition comprises a) a first co-solvent comprising a non-ionic surfactant; b) a second co-solvent comprising an alcoholic ester; and, c) a thickening agent.

60. The method of claim 59, wherein the first co-solvent is selected from the group consisting of polyoxyethylene (20) monoleate, polyoxyethylene (20) sorbitan monooleate, polysorbate 80, palmitate and stearate, wherein the second co-solvent is selected from the group consisting of isopropyl myristate and isopropyl palmitate, and wherein the thickening agent is selected from the group consisting of white wax, cetyl ester wax and glyceryl monosterate.

61. A composition comprising a hapten gel formulation, wherein the composition comprises a) a first co-solvent comprising a non-ionic surfactant, b) a second co-solvent comprising an alcoholic ester, and c) a gelling agent.

62. The composition of claim 61, wherein said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

63. The composition of claim 62, wherein the composition comprises 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol.

64. The composition of any one of claims 61-63, wherein the hapten is DPCP, imiquimod, ingenol mebutate or SADBE.

65. The composition of claim 64, wherein the hapten is DPCP.

66. A composition comprising a hapten ointment formulation, wherein the composition comprises a) a first co-solvent comprising a non-ionic surfactant, b) a second co-solvent comprising an alcoholic ester, and c) a thickening agent.

67. The composition of claim 66, wherein said first co-solvent is selected from the group consisting of polyoxyethylene (20) monoleate, polyoxyethylene (20) sorbitan monooleate, polysorbate 80, palmitate and stearate, wherein said second co-solvent is selected from the group consisting of isopropyl myristate and isopropyl palmitate, and wherein said thickening agent is selected from the group consisting of white wax, cetyl ester wax and glyceryl monosterate.

68. A composition comprising a hapten ointment formulation, wherein the composition comprises 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

69. The composition of any one of claims 66-68, wherein the hapten is DPCP, imiquimod, ingenol mebutate or SADBE.

70. The composition of claim 68 or 69, wherein the hapten is DPCP.

71. The composition of any one of claims 61-70, wherein the dose of DPCP is 0.0000001% to about 1%.

72. A method for treating alopecia areata comprising administering to a subject in need thereof a therapeutically effective amount of a composition comprising a hapten gel formulation, wherein the composition comprises a) a first co-solvent comprising a non-ionic surfactant, b) a second co-solvent comprising an alcoholic ester, and c) a gelling agent.

73. The method of claim 72, wherein said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

74. The method of claim 73, wherein the gel composition comprises 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol.

75. The method of any one of claims 72-74, wherein the hapten is DPCP, imiquimod, ingenol mebutate or SADBE.

76. The method of claim 75, wherein the hapten is DPCP.

77. A method for treating alopecia areata comprising administering to a subject in need thereof a therapeutically effective amount of a composition comprising a hapten ointment formulation, wherein the composition comprises a) a first co-solvent comprising a non-ionic surfactant, b) a second co-solvent comprising an alcoholic ester, and c) a thickening agent.

78. The method of claim 77, wherein said first co-solvent is selected from the group comprising polyoxyethylene (20) monoleate, polyoxyethylene (20) sorbitan monooleate, polysorbate 80, palmitate and stearate, and wherein said second co-solvent is selected from the group comprising of isopropyl myristate and isopropyl palmitate, and wherein said thickening agent is selected from the group comprising of white wax, cetyl ester wax and glyceryl monosterate.

79. A method for treating alopecia areata comprising administering to a subject in need thereof a therapeutically effective amount of a composition comprising a hapten ointment formulation, wherein the ointment is comprised of 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

80. The method of any one of claims 77-79, wherein the hapten is DPCP, imiquimod, ingenol mebutate or SADBE.

81. The method of claim 80, wherein the hapten is DPCP.

82. The method of any one of claims 72-81, wherein the hapten is DPCP and wherein the dose of DPCP is about 0.0000001% to about 1%.

83. A method comprising administering the composition of any one of claims 61-71 to a subject in need thereof.


SEQUENCE LISTING (41 sequences)

SEQ ID NO: 1 - DNA, 822 bp, Homo sapiens
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 2 - DNA, 3216 bp, Homo sapiens
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 3 - DNA, 4050 bp, Homo sapiens
gcagccagagctcagcagggccctggagagatggccacggtcccagcaccggggaggactggagagcgcgcgctgccaccgccccatgtctcagccagggcttccttcctcggctccaccctgtggatgtaatggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtagacagatggccagggtgggaggcaggcagctgcctgctctgcgccgagcctcagaaggaccctgttgagggtcctcagtccactgctgaggacactcagtgtccagttgcagctggacttctccacccggatggcccccacccagtcctgcacacttggtccatccatttccaaacctccactgctgctcccgggtcctgctgcccgagccaggaactgtgtgtgttgcaggggggcagtaactccccaactccctcgttaatcacaggatcccacgaatttaggctcagaagcatcgctcctctccagccctgcagctattcaccaatatcagtcctcgcggctctccagggctccctgccctgacctcttccctgggttttctgccccagcctcctccttccctcccctccccgtccacagggcagcctgagcgtgctttccaaaacccaaatatggccacgctccccctcggttcaaaaccttgcacaggtcccactgccctcagccccacttctcagcctggtacttgtacctccggtgtcgtgtggggacatccccttctgcaatcctccctaccgtcctcctgagccactcagagctccctcacaccccctctgttgcacatgctattccctggggctgctgtgcgctccccctcatctaggtgacaaacttccctgactcttcaagtgccggttttgcttctcctggagggaagcactgcctcccttaatctgccagaaacttctagcgtcagtgctggagggagaagctgtcagggacccagggcgcctggagaaagaggccctgttactattcctttgggatctctgaggcctcagagtgcttggctgctgtatctttaatgctggggcccaagtaagggcacagatccccccacaaagtggatgcctgctgcatcttcccacagtggcttcacagacccacaagagaagctgatggggagtaaaccctggagtccgaggcccaggcagcagccccgcctagtggtgggccctgatgctgccaggcctgggacctcccactgccccctccactggaggggtctcctctgcagctcagggactggcacactggcctccagaagggcagctccacagggcagggcctcattatttttcactgccccagacacagtgcccaacaccccgtcgtataccctggatgaacgaattaattacctggcaccacctcgtctgggctccctgcgcctgacattcacacagagaggcagagtcccgtgcccattaggtctggcatgccccctcctgcaaggggctcaaccccctaccccgacccctccacgtatctttcctaggcagatcacgttgcaatggctcaaacaacattccaccccagcaggacagtgaccccagtcccagctaactctgacctgggagccctcaggcacctgcacttacaggccttgctcacagctgattgggcacctgaccacacgcccccacaggctctgaccagcagcctatgagggggtttggcaccaagctctgtccaatcaggtaggctgggcctgaactagccaatcagatcaactctgtcttgggcgtttgaactcagggagggaggcccttgggagcaggtgcttgtggacaaggctccacaagcgttgagccttggaaaggtagacaagcgttgagccactaagcagaggaccttgggttcccaatacaaaaatacctactgctgagagggctgctgaccatttggtcaggattcctgttgcctttatatccaaaataaactcccctttcttgaggttgtctgagtcttgggtctatgccttgaaaaaagctgaattattggacagtctcacctcctgccatagggtcctgaatgtttcagaccacaaggggctccacacctttgctgtgtgttctggggcaacctactaatcctctctgcaagtcggtctccttatccccccaaatggaaattgtatttgccttctccactttgggaggctcccacttcttgggagggttacattttttaagtcttaatcatttgtgacatatgtatctatacatccgtatcttttaatgatccgtgtgtaccatctttgtgattatttccttaatattttttctttaagtcagttcattttcgttgaaatacatttatttaaagaaaaatctttgttactctgtaaatgaaaaaacccattttcgctataaataaaaggtaactgtacaaaataagtacaatgcaacaaaaaaaaa

SEQ ID NO: 4 - DNA, 1560 bp, Homo sapiens
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaaaaaaaaaaaaaaaaaaaaaa

SEQ ID NO: 5 - DNA, 2333 bp, Homo sapiens
gttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgttttcaactagtggccccactgtgtccggaattgatgggttcttggtctcactgacttcaagaatgaagccgcggaccctcgcggtgagtgttacagctcttaaggtggcgcatctggagtttgttccttctgatgttcggatgtgttcggagtttcttccttctggtgggttcgtggtctcgctggctcaggagtgaagctacagaccttcgcggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggatgcagctaatatacccagttggcccaaagcacctaacctatagttatataatctgactctcagttcagttttactctactaatgccttcatggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaaaaaaaaaaaa

SEQ ID NO: 6 - DNA, 1841 bp, Homo sapiens
agctgcagcaggaattcggcgaagtggcggagctggggccccagcgggcgccgggggccgcgggagccagcaggtggcgggggctgcgctccgcccgggccagagcgcaccaggcaggtgcccgcgcctccgcaccgcggcgacacctccgcgggcactcacccaggccggccgctcacaaccgagcgcagggccgcggagggagaccaggaaagccgaaggcggagcagctggaggcgaccagcgccgggcgaggtcaagtggatccgagccgcagagagggctggagagagtctgctctccgatgactttgcccactctcttcgcagtggggacaccggaccgagtgcacactggaggtcccagagcacgacgagcgcggaggaccgggaggctcccgggcttgcgtgggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagactgagcaggatttttatttcattacagtgtgagctgcctggaatacatgtggtaatgaaataaaaaccctgccccgaatcttccgtccctcatcctaactttcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgaaacaaaataaaacatctattttcaatatttttaaaatgcaaaaaaaaaaa

SEQ ID NO: 7 - DNA, 1450 bp, Homo sapiens
tttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtgaaaaaaaa

SEQ ID NO: 8 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 9 - DNA, 2800 bp, Homo sapiens
ctctttcactttgacttgccttagggatgggctgtgacactttactttttttcttttttcttttttttcagtcttttctccttgctcagcttcaatgtgttccggagtggggacggggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagagcatcccgtgcagcccacagagacccaagttaccctcagtggcctgcgggctggtgtagcctacacggtgcaggtgcgagcagacacagcgtggctgaggggtgtctggagccagccccagcgcttcagcatcgaagtgcaggtttctgattggctcatcttcttcgcctccctggggagcttcctgagcatccttctcgtgggcgtccttggctaccttggcctgaacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtgatcgttgaggctcagagagggtgagtgactcgcccgaggctacgtagcacacacaggagtcacatttggacccaaataacccagagctcctccaggctccagtgcacctgcctcctctctgccccgtgcctgttgccacccatcctgcgggggaaccctagatgctgccatgaaatggaagctgctgcaccctgctgggcctggcatccgtggggcaggagcagaccctgccatttacctgttctggcgtagaatggactgggaatgggggcaaggggggctcagatggatccctggaccctgggctgggcatccacccccaggagcactggatggggagtctggactcaagggctccctgcagcattgcggggtcttgtagcttggaggatccaggcatatagggaagggggctgtaaactttgtgggaaaaatgacggtcctcccatcccaccccccaccccaccctcacccccctataaaatgggggtggtgataatgaccttacacagctgttcaaaatcatcgtaaatgagcctcctcttgggtatttttttcctgtttgaagcttgaatgtcctgctcaaaatctcaaaacacgagccttggaattcaaaaaaaaaaaaaaaaaaa

SEQ ID NO: 10 - DNA, 4040 bp, Homo sapiens
tgcagagcacagagaaaggacatctgcgaggaaagttccctgatggctgtcaacaaagtgccacgtctctatggctgtgaacgctgagcacacgattttatcgcgcctatcatatcttggtgcataaacgcacctcacctcggtcaacccttgctccgtcttatgagacaggctttattatccgcattttatatgaggggaaactgacggtggagagagaattatcttgctcaaggcgacacagcagagcccacaggtggcagaatcccacccgagcccgcttcgacccgcggggtggaaaccacgggcgcccgcccggctgcgcttccagagctgaactgagaagcgagtcctctccgccctgcggccaccgcccagccccgacccccgccccggcccgatcctcactcgccgccagctccccgcgcccaccccggagttggtggcgcagaggcgggaggcggaggcgggagggcgggcgctggcaccgggaacgcccgagcgccggcagagagcgcggagagcgcgacacgtgcggcccagagcaccggggccacccggtccccgcaggcccgggaccgcgcccgctggcaggcgacacgtggaagaatacggagttctataccagagttgattgttgatggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtatcaatagtgatgaaattcaaatatgtggagcagagatcttcgttggtgttgctccagaacagcctcaaaatttatcctgcatacagaagggagaacaggggactgtggcctgcacctgggaaagaggacgagacacccacttatacactgagtatactctacagctaagtggaccaaaaaatttaacctggcagaagcaatgtaaagacatttattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttctgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctgagtggtgaggcttcaagccttaaagtcagtgtgccctcaaccagcacagcctgccccaattcccccagcccctgctccagcagctgtcatctctgggtgccaccatcggtctggctgcagctagaggacaggcaagccagctctgggggagtcttaggaactgggagttggtcttcactcagatgcctcatcttgcctttcccagggccttaaaattacatccttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagccttcaaaacaaatcaacctagttctatagatgaagacataaaagacactggtaaacaccaaggtaaaagggcccccaaggtggtcatgactggtctcatttgcagaagtctaagaatgtacctttttctggccgggcgtggtagctcatgcctgtaatcccagcactttgggaggctga

SEQ ID NO: 11 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 12 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 13 - DNA, 4900 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaaaaaaaaaaaaaaaaa

SEQ ID NO: 14 - DNA, 913 bp, Homo sapiens
ccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtggtggcaggacaagagaaggcattgagctttttctttcattttcctattaaaaaatacaaaaatca

SEQ ID NO: 15 - DNA, 1320 bp, Homo sapiens
cggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccaccctgca

SEQ ID NO: 16 - DNA, 3083 bp, Homo sapiens
gccaggtgctcccgccttccaccctccgccctcctccctcccctgggccctgctccctgccctcctgggcagccagggcagccaggacggcaccaagggagctgccccatggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacctggggtctaaccaaaaatggatgggatgaggatgagaggctggagataattgttttatgggatttgggtgtgggactagggtacaatgaaggccaagagcatctcagacatagagttaaaactcaaacctcttatgtgcactttaaagatagactttaggggctggcacaaatctgatcagagacacatatccatacacaggtgaaacacatacagactcaacagcaatcatgcagttccagagacacatgaacctgacacaatctctcttatccttgaggccacagcttggaggagcctagaggcctcaggggaaagtcccaatcctgagggaccctcccaaacatttccatggtgctccagtccactgatcttgggtctggggtgatccaaataccaccccagctccagctgtcttctaccactagaagacccaagagaagcagaagtcgctcgcactggtcagtcggaaggcaagatcagatcctggaggactttcctggcctgcccgccagccctgctcttgttgtggagaaggaagcagatgtgatcacatcaccccgtcattgggcaccgctgactccagcatggaggacaccagggagcagggcctgggcctgtttccccagctgtgatctgcccagaacctctcttggcttcataaacagctgtgaaccctcccctgagggattaacagcaatgatgggcagtcgtggagttgggggggttgggggtgggattgtgtcctctaaggggacgggttcatctgagtaaacataaaccccaacttgtgccattctttataaaatgattttaaaggcaaaaaaaaaaaaaaaaaaaa

SEQ ID NO: 17 - DNA, 2589 bp, Homo sapiens
cggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttaaaaaaaaaaaaaaaaa

SEQ ID NO: 18 - DNA, 735 bp, Homo sapiens
atggcagcggccgccagccccgcgatccttccgcgcctcgcgattcttccgtacctgctattcgactggtccgggacggggcgggccgacgctcactctctctggtataacttcaccatcattcatttgcccagacatgggcaacagtggtgtgaggtccagagccaggtggatcagaagaattttctctcctatgactgtggcagtgacaaggtcttatctatgggtcacctagaagagcagctgtatgccacagatgcctggggaaaacaactggaaatgctgagagaggtggggcagaggctcagactggaactggctgacactgagctggaggatttcacacccagtggacccctcacgctgcaggtcaggatgtcttgtgagtgtgaagccgatggatacatccgtggatcttggcagttcagcttcgatggacggaagttcctcctctttgactcaaacaacagaaagtggacagtggttcacgctggagccaggcggatgaaagagaagtgggagaaggatagcggactgaccaccttcttcaagatggtctcaatgagagactgcaagagctggcttagggacttcctgatgcacaggaagaagaggctggaacccacagcaccacccaccatggccccaggcttagctcaacccaaagccatagccaccaccctcagtccctggagcttcctcatcatcctctgcttcatcctccctggcatctga

SEQ ID NO: 19 - DNA, 2278 bp, Homo sapiens
gtatcatttcagtgaaggtcactccagtctttcatggaggccaaactaagggtgtaaattaggatcctcactgaagtggcgggaccctaagaggctttttcctggccccttagttgtgggttttcctgcgggcggcgcagccggtttccatcagaaccgcccagaggcggacgctgccttcctggggtgacggagcagcaggaagcgttttcggatcctggaatacgtgggcggcccgtgggaggggctgaggcgcagtttcctactcacccggatccgaatcctccgcggtgctgtttcaagagagccggattccagatcacgctccagcccggactcggaattcctgccctgcgggtctgcattttcataacgggcaggtgtgagtgccctgcagctggagaccagaagcctgaaggcagctcggccctccccagcccacagcgccgttattccgtttctatatcagtaaacacatttcattttccgtagaccagggcggggtgacgggtgatcccagtcctcgcagtgaattccgggcagcaaaattcaaaacacatgcggccaaggccgggcacggtggttcacgcctgtaatcccagcactttgggaggtcgaggcgggcgatcacctgaggtcgggagctcgagaccaacctgaccaacatggggaaatcccgtctctactaaaaatataaaattagacgggcttggtggtgaatgcctgtaatcccagctagtcgggaggctgaggcaggagaatcgcttaaaccttggaggcggaggttgcggtgagccgagatcgcgccattgcacttcagcctgggcaacaagagggaaaactccgtcgcaaaaactttcgggggcggagcggagccccgccctgggttatgtaagcgaccgcgctgggccgtttctctttcttttccggaccctgcagtggcgcctaaagtctgagagagggaagtcgcctctgtgctcgtgagtgcatggggtataagagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccggcgtagtcctgaggagaacagtgccccccatggtgaatgtcacccgcagcgaggcctcagagggcaacatcaccgtgacatgcagggcttccagcttctatccccggaatatcatactgacctggcgtcaggatggggtatctttgagccacgacacccagcagtggggggatgtcctgcctgatgggaatggaacctaccagacctgggtggccaccaggatttgccgaggagaggagcagaggttcacctgctacatggaacacagcgggaatcacagcactcaccctgtgccctctgggaaagtgctggtgcttcagagtcattggcagacattccatgtttctgctgttgctgctggctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagctcttgggtccactggctccactgagggcacctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatattaaaaaaaaaaaaaaaaaa

SEQ ID NO: 20 - DNA, 1606 bp, Homo sapiens
actaagtatctccactttcaattctagatcaggaactgaggacatatctaaattttctagttttatagaaggcttttatccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtaaagatgatcaaccatctcaataaaagccaggaacagagaagagattacaccagcggtaacactgccaactgagactaaaggaaacaaacaaaaacaggacaaaatgaccaaagactgtcagatttcttagactccacaggaccaaaccatagaacaatttcactgcaaacatgcatgattctccaagacaaaagaagagagatcctaaaggcaattcagatatccccaaggctgcctctcccaccacaagcccagagtggatgggctgggggaggggtgctgttttaatttctaaaggtaggaccaacacccaggggatcagtgaaggaagagaaggccagcagatcactgagagtgcaaccccaccctccacaggaaattgcctcatgggcagggccacagcagagagacacagcatgggcagtgccttccctgcctgtgggggtcatgctgccacttttaatgggtcctccacccaacggggtcagggaggtggtgctgccccagtgggccatgattatcttaaaggcattattctccagccttaagtaagatcttaggacgtttcctttgctatgatttgtacttgcttgagtcccatgactgtttctcttcctctctttcttccttttggaatagtaatatccatcctatgtttgtcccactattgtattttggaagcacataacttgtttggtttcacaggttcacagttaagaaggaattttgcctctgaataaatagaatcttgagtctcatgcaaaaaaaaaaaaaaaaaa

SEQ ID NO: 21 - DNA, 913 bp, Homo sapiens
cgcgcctgcgcagtggaggcggcccaggcccgccttccgcagggtgtcgccgctgtgccgctagcggtgccccgcctgctgcggtggcaccagccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggccatctccttggttaaaaaaaaa

SEQ ID NO: 22 - DNA, 5053 bp, Homo sapiens
tgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagactttacatgca

SEQ ID NO: 23 - DNA, 5285 bp, Homo sapiens
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgtcccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtcagttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaattaagtataaggggttgttcgttgttgtcatttgttatagtgctactccactttagacaccatagctaaaataaaatatggtgggttttgtgtgtgtgtgtgtgtgtgtgtgtgtgtgtgtgtgtgtgttatttatacaaaacttaaaatacttgctgttttgattaaaaagaaaatagtttcttacttta

SEQ ID NO: 24 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 25 - RNA, 13 nt, Artificial Sequence
  Synthetic Polynucleotide
gcaccuuucuaga

SEQ ID NO: 26 - RNA, 19 nt, Artificial Sequence
  Synthetic Polynucleotide
ucuagaaaggugcaaacau

SEQ ID NO: 27 - RNA, 13 nt, Artificial Sequence
  Synthetic Polynucleotide
  misc_feature (2)..(2): Modified with 2′Ome
  misc_feature (4)..(10): Modified with 2′Ome
  misc_feature (11)..(13): Phosphorothioate internucleotide bond
  misc_feature (12)..(13): Modified with 2′Ome
  misc_feature (13)..(13): Modified with TEG-Chl
gcaccuuucuaga

SEQ ID NO: 28 - RNA, 19 nt, Artificial Sequence
  Synthetic Polynucleotide
  misc_feature (1)..(1): Modified with P
  misc_feature (1)..(1): Modified with 2′Ome
  misc_feature (2)..(3): Modified with 2′fluoro
  misc_feature (6)..(6): Modified with 2′Ome
  misc_feature (8)..(8): Modified with 2′Ome
  misc_feature (11)..(11): Modified with 2′fluoro
  misc_feature (13)..(13): Modified with 2′Ome
  misc_feature (13)..(19): Phosphorothioate internucleotide bond
  misc_feature (17)..(17): Modified with 2′Ome
ucuagaaaggugcaaacau

SEQ ID NO: 29 - RNA, 19 nt, Artificial Sequence
  Synthetic Polynucleotide
  misc_feature (1)..(1): Modified with P
  misc_feature (1)..(1): Modified with 2′Ome
  misc_feature (2)..(2): Modified with 2′fluoro
  misc_feature (5)..(6): Modified with 2′fluoro
  misc_feature (10)..(10): Modified with 2′Ome
  misc_feature (13)..(19): Phosphorothioate internucleotide bond
  misc_feature (14)..(14): Modified with 2′fluoro
  misc_feature (15)..(15): Modified with 2′Ome
  misc_feature (16)..(16): Modified with 2′fluoro
  misc_feature (17)..(18): Modified with 2′Ome
ucaacuagaaaggugcaaa

SEQ ID NO: 30 - RNA, 14 nt, Artificial Sequence
  Synthetic Polynucleotide
uugcaccuuucuaa

SEQ ID NO: 31 - RNA, 20 nt, Artificial Sequence
  Synthetic Polynucleotide
uuagaaaggugcaaacaagg

SEQ ID NO: 32 - RNA, 14 nt, Artificial Sequence
  Synthetic Polynucleotide
  misc_feature (1)..(2): Modified with 2′Ome
  misc_feature (4)..(4): Modified with 2′Ome
  misc_feature (6)..(14): Modified with 2′Ome
  misc_feature (12)..(14): Phosphorothioate internucleotide bond
  misc_feature (14)..(14): Modified with TEG-Chl
uugcaccuuucuaa

SEQ ID NO: 33 - RNA, 20 nt, Artificial Sequence
  Synthetic Polynucleotide
  misc_feature (1)..(1): Modified with P
  misc_feature (1)..(1): Modified with 2′Ome
  misc_feature (2)..(2): Modified with 2′fluoro
  misc_feature (6)..(6): Modified with 2′Ome
  misc_feature (10)..(10): Modified with 2′fluoro
  misc_feature (12)..(12): Modified with 2′fluoro
  misc_feature (13)..(15): Modified with 2′Ome
  misc_feature (14)..(20): Phosphorothioate internucleotide bond
  misc_feature (16)..(16): Modified with 2′fluoro
  misc_feature (17)..(19): Modified with 2′Ome
uuagaaaggugcaaacaagg

SEQ ID NO: 34 - RNA, 13 nt, Artificial Sequence
  Synthetic Polynucleotide
gugaccaaaagua

SEQ ID NO: 35 - RNA, 19 nt, Artificial Sequence
  Synthetic Polynucleotide
uacuuuuggucacacucuc

SEQ ID NO: 36 - RNA, 13 nt, Artificial Sequence
  Synthetic Polynucleotide
  misc_feature (2)..(2): Modified with 2′Ome
  misc_feature (5)..(6): Modified with 2′Ome
  misc_feature (11)..(13): Phosphorothioate internucleotide bond
  misc_feature (12)..(13): Modified with 2′Ome
  misc_feature (13)..(13): Modified with TEG-Chl
gugaccaaaagua

SEQ ID NO: 37 - RNA, 19 nt, Artificial Sequence
  Synthetic Polynucleotide
  misc_feature (1)..(1): Modified with P
  misc_feature (1)..(1): Modified with 2′Ome
  misc_feature (3)..(7): Modified with 2′fluoro
  misc_feature (10)..(10): Modified with 2′fluoro
  misc_feature (11)..(11): Modified with 2′Ome
  misc_feature (13)..(13): Modified with 2′Ome
  misc_feature (13)..(19): Phosphorothioate internucleotide bond
  misc_feature (15)..(18): Modified with 2′Ome
uacuuuuggucacacucuc

SEQ ID NO: 38 - RNA, 13 nt, Artificial Sequence
  Synthetic Polynucleotide
ccuuucuaguuga

SEQ ID NO: 39 - RNA, 19 nt, Artificial Sequence
  Synthetic Polynucleotide
ucaacuagaaaggugcaaa

SEQ ID NO: 40 - RNA, 13 nt, Artificial Sequence
  Synthetic Polynucleotide
  misc_feature (1)..(7): Modified with 2′Ome
  misc_feature (10)..(13): Modified with 2′Ome
  misc_feature (11)..(13): Phosphorothioate internucleotide bond
  misc_feature (13)..(13): Modified with TEG-Chl
ccuuucuaguuga

SEQ ID NO: 41 - RNA, 19 nt, Artificial Sequence
  Synthetic Polynucleotide
  misc_feature (1)..(1): Modified with P
  misc_feature (1)..(1): Modified with 2′Ome
  misc_feature (2)..(3): Modified with 2′fluoro
  misc_feature (6)..(6): Modified with 2′Ome
  misc_feature (8)..(8): Modified with 2′Ome
  misc_feature (11)..(11): Modified with 2′fluoro
  misc_feature (13)..(13): Modified with 2′fluoro
  misc_feature (13)..(19): Phosphorothioate internucleotide bond
  misc_feature (14)..(16): Modified with 2′Ome
  misc_feature (17)..(17): Modified with 2′fluoro
  misc_feature (18)..(18): Modified with 2′Ome
ucuagaaaggugcaaacau